SEQUENCE LISTING (165 sequences)

SEQ ID NO: 1 - protein, 742 aa, Homo sapiens [sequence omitted]

SEQ ID NO: 2 - protein, 493 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSTTLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 3 - protein, 361 aa, Homo sapiens [sequence omitted]

SEQ ID NO: 4 - DNA, 336 bp, Artificial Sequence
  Synthetically generated oligonucleotide
gacatccagatgacccagtctccactctccctggccgtcacccttggacagccggcctccatctcctgcaggtctagtgaaagcctcgtgtacagtgatggaaacacctacttgggttggtttcagcagaggccaggccaatctccacggcgcctactttataaggtttctaaccgggactctggggtcccagacagattcagcggcagtgggtcaggcactgatttcacactgcacatcagcagggtggaggctgaagatgttggggtttattactgcatgcattctatacgctggccgtggacgttcggccaagggaccacggtggaaatcaag

SEQ ID NO: 5 - protein, 112 aa, Artificial Sequence
  Synthetically generated peptide
DIQMTQSPLSLAVTLGQPASISCRSSESLVYSDGNTYLGWFQQRPGQSPRRLLYKVSNRDSGVPDRFSGSGSGTDFTLHISRVEAEDVGVYYCMHSIRWPWTFGQGTTVEIK

SEQ ID NO: 6 - DNA, 357 bp, Artificial Sequence
  Synthetically generated oligonucleotide
gaagttcaattgttagagtctggtggcggtcttgttcagcctggtggttctttacgtctttcttgcgctgcttccggattcactttctctccttacactatggcttgggttcgccaagctcctggtaaaggtttggagtgggtttcttctatctatccttctggtggcactactccttatgctgactccgttaaaggtcgcttcactatctctagagacaactctaagaatactctctacttgcagatgaacagcttaagggctgaggacactgcagtctactattgtgcgagacattttactgtgtatgatggttttgatttgtggggccgagggacaatggtcaccgtctcaagc

SEQ ID NO: 7 - protein, 119 aa, Artificial Sequence
  Synthetically generated peptide
EVQLLESGGGLVQPGGSLRLSCAASGFTFSPYTMAWVRQAPGKGLEWVSSIYPSGGTTPYADSVKGRFTISRDNSKNTLYLQMNSLRAEDTAVYYCARHFTVYDGFDLWGRGTMVTVSS

SEQ ID NO: 8 - DNA, 324 bp, Artificial Sequence
  Synthetically generated oligonucleotide
gacatccagatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgaacgttcggccaagggaccaaggtggaaatcaaa

SEQ ID NO: 9 - protein, 108 aa, Artificial Sequence
  Synthetically generated peptide
DIQMTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 10 - DNA, 360 bp, Artificial Sequence
  Synthetically generated oligonucleotide
gaagttcaattgttagagtctggtggcggtcttgttcagcctggtggttctttacgtctttcttgcgctgcttccggattcactttctctcattacggtatgtcttgggttcgccaagctcctggtaaaggtttggagtgggtttcttggatcggtccttctggtggcgctactctttatgctgactccgttaaaggtcgcttcactatctctagagacaactctaagaatactctctacttgcagatgaacagcttaagggctgaggacactgcagtctactattgtgcgaaaggaaggtggaataggggtggcgcctttgacaactggggccagggaaccctggtcaccgtctcaagc

SEQ ID NO: 11 - protein, 120 aa, Artificial Sequence
  Synthetically generated peptide
EVQLLESGGGLVQPGGSLRLSCAASGFTFSHYGMSWVRQAPGKGLEWVSWIGPSGGATLYADSVKGRFTISRDNSKNTLYLQMNSLRAEDTAVYYCAKGRWNRGGAFDNWGQGTLVTVSS

SEQ ID NO: 12 - DNA, 333 bp, Artificial Sequence
  Synthetically generated oligonucleotide
gacatccagatgacccagtctccactctccctgcccgtcacccctggagggccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctgcaaccgtacacttttggccaggggaccaagctggagatcaaa

SEQ ID NO: 13 - protein, 111 aa, Artificial Sequence
  Synthetically generated peptide
DIQMTQSPLSLPVTPGGPASISCRSSQSLLHSNGYNYLDWYLQKPGQSPQLLIYLGSNRASGVPDRFSGSGSGTDFTLKISRVEAEDVGVYYCMQALQPYTFGQGTKLEIK

SEQ ID NO: 14 - DNA, 369 bp, Artificial Sequence
  Synthetically generated oligonucleotide
gaagttcaattgttagagtctggtggcggtcttgttcagcctggtggttctttacgtctttcttgcgctgcttccggattcactttctctccttaccttatgtcttgggttcgccaagctcctggtaaaggtttggagtgggtttcttctatctattcttctggtggccttactgattatgctgactccgttaaaggtcgcttcactatctctagagacaactctaagaatactctctacttgcagatgaacagcttaagggctgaggacactgcagtctaccattgtgcgagagacggttactatgatagtagtggttacgagggttttgactactggggccagggaaccctggtcaccgtctcaagc

SEQ ID NO: 15 - protein, 123 aa, Artificial Sequence
  Synthetically generated peptide
EVQLLESGGGLVQPGGSLRLSCAASGFTFSPYLMSWVRQAPGKGLEWVSSIYSSGGLTDYADSVKGRFTISRDNSKNTLYLQMNSLRAEDTAVHYCARDGYYDSSGYEGFDYWGQGTLVTVSS

SEQ ID NO: 16 - protein, 16 aa, Artificial Sequence
  Synthetically generated peptide
RSSESLVYSDGNTYLG

SEQ ID NO: 17 - protein, 7 aa, Artificial Sequence
  Synthetically generated peptide
KVSNRDS

SEQ ID NO: 18 - protein, 9 aa, Artificial Sequence
  Synthetically generated peptide
MHSIRWPWT

SEQ ID NO: 19 - protein, 23 aa, Artificial Sequence
  Synthetically generated peptide
DIQMTQSPLSLAVTLGQPASISC

SEQ ID NO: 20 - protein, 15 aa, Artificial Sequence
  Synthetically generated peptide
WFQQRPGQSPRRLLY

SEQ ID NO: 21 - protein, 32 aa, Artificial Sequence
  Synthetically generated peptide
GVPDRFSGSGSGTDFTLHISRVEAEDVGVYYC

SEQ ID NO: 22 - protein, 10 aa, Artificial Sequence
  Synthetically generated peptide
FGQGTTVEIK

SEQ ID NO: 23 - protein, 5 aa, Artificial Sequence
  Synthetically generated peptide
PYTMA

SEQ ID NO: 24 - protein, 17 aa, Artificial Sequence
  Synthetically generated peptide
SIYPSGGTTPYADSVKG

SEQ ID NO: 25 - protein, 9 aa, Artificial Sequence
  Synthetically generated peptide
HFTVYDGFD

SEQ ID NO: 26 - protein, 30 aa, Artificial Sequence
  Synthetically generated peptide
EVQLLESGGGLVQPGGSLRLSCAASGFTFS

SEQ ID NO: 27 - protein, 14 aa, Artificial Sequence
  Synthetically generated peptide
WVRQAPGKGLEWVS

SEQ ID NO: 28 - protein, 32 aa, Artificial Sequence
  Synthetically generated peptide
RFTISRDNSKNTLYLQMNSLRAEDTAVYYCAR

SEQ ID NO: 29 - protein, 12 aa, Artificial Sequence
  Synthetically generated peptide
LWGRGTMVTVSS

SEQ ID NO: 30 - protein, 12 aa, Artificial Sequence
  Synthetically generated peptide
RASQSVSSSYLA

SEQ ID NO: 31 - protein, 7 aa, Artificial Sequence
  Synthetically generated peptide
GASSRAT

SEQ ID NO: 32 - protein, 9 aa, Artificial Sequence
  Synthetically generated peptide
QQYGSSPRT

SEQ ID NO: 33 - protein, 23 aa, Artificial Sequence
  Synthetically generated peptide
DIQMTQSPGTLSLSPGERATLSC

SEQ ID NO: 34 - protein, 15 aa, Artificial Sequence
  Synthetically generated peptide
WYQQKPGQAPRLLIY

SEQ ID NO: 35 - protein, 32 aa, Artificial Sequence
  Synthetically generated peptide
GIPDRFSGSGSGTDFTLTISRLEPEDFAVYYC

SEQ ID NO: 36 - protein, 10 aa, Artificial Sequence
  Synthetically generated peptide
FGQGTKVEIK

SEQ ID NO: 37 - protein, 5 aa, Artificial Sequence
  Synthetically generated peptide
HYGMS

SEQ ID NO: 38 - protein, 17 aa, Artificial Sequence
  Synthetically generated peptide
WIGPSGGATLYADSVKG

SEQ ID NO: 39 - protein, 10 aa, Artificial Sequence
  Synthetically generated peptide
GRWNRGGAFD

SEQ ID NO: 40 - protein, 30 aa, Artificial Sequence; identical to SEQ ID NO: 26
  Synthetically generated peptide

SEQ ID NO: 41 - protein, 14 aa, Artificial Sequence
  Synthetically generated peptide
WVRQAPGKGLEWVS

SEQ ID NO: 42 - protein, 32 aa, Artificial Sequence
  Synthetically generated peptide
RFTISRDNSKNTLYLQMNSLRAEDTAVYYCAK

SEQ ID NO: 43 - protein, 12 aa, Artificial Sequence
  Synthetically generated peptide
NWGQGTLVTVSS

SEQ ID NO: 44 - protein, 16 aa, Artificial Sequence
  Synthetically generated peptide
RSSQSLLHSNGYNYLD

SEQ ID NO: 45 - protein, 7 aa, Artificial Sequence
  Synthetically generated peptide
LGSNRAS

SEQ ID NO: 46 - protein, 8 aa, Artificial Sequence
  Synthetically generated peptide
MQALQPYT

SEQ ID NO: 47 - protein, 23 aa, Artificial Sequence
  Synthetically generated peptide
DIQMTQSPLSLPVTPGGPASISC

SEQ ID NO: 48 - protein, 15 aa, Artificial Sequence
  Synthetically generated peptide
WYLQKPGQSPQLLIY

SEQ ID NO: 49 - protein, 32 aa, Artificial Sequence
  Synthetically generated peptide
GVPDRFSGSGSGTDFTLKISRVEAEDVGVYYC

SEQ ID NO: 50 - protein, 10 aa, Artificial Sequence
  Synthetically generated peptide
FGQGTKLEIK

SEQ ID NO: 51 - protein, 5 aa, Artificial Sequence
  Synthetically generated peptide
PYLMS

SEQ ID NO: 52 - protein, 17 aa, Artificial Sequence
  Synthetically generated peptide
SIYSSGGLTDYADSVKG

SEQ ID NO: 53 - protein, 13 aa, Artificial Sequence
  Synthetically generated peptide
DGYYDSSGYEGFD

SEQ ID NO: 54 - protein, 30 aa, Artificial Sequence; identical to SEQ ID NO: 26
  Synthetically generated peptide

SEQ ID NO: 55 - protein, 14 aa, Artificial Sequence
  Synthetically generated peptide
WVRQAPGKGLEWVS

SEQ ID NO: 56 - protein, 32 aa, Artificial Sequence
  Synthetically generated peptide
RFTISRDNSKNTLYLQMNSLRAEDTAVYHCAR

SEQ ID NO: 57 - protein, 12 aa, Artificial Sequence
  Synthetically generated peptide
YWGQGTLVTVSS

SEQ ID NO: 58 - DNA, 336 bp, Artificial Sequence
  Synthetically generated oligonucleotide
gacatccagatgacccagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcccactttcggcggagggaccaaggtggagatcaaa

SEQ ID NO: 59 - protein, 112 aa, Artificial Sequence
  Synthetically generated peptide
DIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGGGTKVEIK

SEQ ID NO: 60 - DNA, 348 bp, Artificial Sequence
  Synthetically generated oligonucleotide
gaagttcaattgttagagtctggtggcggtcttgttcagcctggtggttctttacgtctttcttgcgctgcttccggattcactttctctgagtacggtatgggttgggttcgccaagctcctggtaaaggtttggagtgggtttcttctatcgtttcttctggtggctttactttttatgctgactccgttaaaggtcgcttcactatctctagagacaactctaagaatactctctacttgcagatgaacagcttaagggctgaggacactgcagtctactattgtgcgagaggcactcgtacagtaaccaactggggccagggagccctggtcaccgtctcaagc

SEQ ID NO: 61 - protein, 116 aa, Artificial Sequence
  Synthetically generated peptide
EVQLLESGGGLVQPGGSLRLSCAASGFTFSEYGMGWVRQAPGKGLEWVSSIVSSGGFTFYADSVKGRFTISRDNSKNTLYLQMNSLRAEDTAVYYCARGTRTVTNWGQGALVTVSS

SEQ ID NO: 62 - DNA, 336 bp, Artificial Sequence
  Synthetically generated oligonucleotide
gacatccagatgacccagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaaccccttggacttttggccaggggaccaagctggagatcaaa

SEQ ID NO: 63 - protein, 112 aa, Artificial Sequence
  Synthetically generated peptide
DIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKLEIK

SEQ ID NO: 64 - DNA, 354 bp, Artificial Sequence
  Synthetically generated oligonucleotide
gaagttcaattgttagagtctggtggcggtcttgttcagcctggtggttctttacgtctttcttgcgctgcttccggattcactttctctctttaccgtatgcgttgggttcgccaagctcctggtaaaggtttggagtgggtttcttctatctctccttctggtggcattactgagtatgctgactccgttaaaggtcgcttcactatctctagagacaactctaagaatactctctacttgcagatgaacagcttaagggctgaggacactgcagtctactattgtgcgctagacgtgggggtgggagctgctgactactggggccagggaaccctggtcaccgtctcaagc

SEQ ID NO: 65 - protein, 118 aa, Artificial Sequence
  Synthetically generated peptide
EVQLLESGGGLVQPGGSLRLSCAASGFTFSLYRMRWVRQAPGKGLEWVSSISPSGGITEYADSVKGRFTISRDNSKNTLYLQMNSLRAEDTAVYYCALDVGVGAADYWGQGTLVTVSS

SEQ ID NO: 66 - DNA, 336 bp, Artificial Sequence
  Synthetically generated oligonucleotide
gacatccagatgacccagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcggagtggaggctgaggatgttggggtttattactgcatgcaagctctacaaactgggtacacttttggccaggggaccaagctggagatcaaa

SEQ ID NO: 67 - protein, 112 aa, Artificial Sequence
  Synthetically generated peptide
DIQMTQSPLSLPVTPGEPASISCRSSQSLLHSNGYNYLDWYLQKPGQSPQLLIYLGSNRASGVPDRFSGSGSGTDFTLKISGVEAEDVGVYYCMQALQTGYTFGQGTKLEIK

SEQ ID NO: 68 - DNA, 354 bp, Artificial Sequence
  Synthetically generated oligonucleotide
gaagttcaattgttagagtctggtggcggtcttgttcagcctggtggttctttacgtctttcttgcgctgcttccggattcactttctctaagtacactatgtggtgggttcgccaagctcctggtaaaggtttggagtgggtttcttctatctggtcttctggtggctttactcgttatgctgactccgttaaaggtcgcttcactatctctagagacaactctaagaatactctctacttgcagatgaacagcttaagggctgaggacactgcagtctactattgtgcgggacgtagtgggagctaccccgctgatatctggggccaagggacaatggtcaccgtctcaagc

SEQ ID NO: 69 - protein, 118 aa, Artificial Sequence
  Synthetically generated peptide
EVQLLESGGGLVQPGGSLRLSCAASGFTFSKYTMWWVRQAPGKGLEWVSSIWSSGGFTRYADSVKGRFTISRDNSKNTLYLQMNSLRAEDTAVYYCAGRSGSYPADIWGQGTMVTVSS

SEQ ID NO: 70 - DNA, 336 bp, Artificial Sequence
  Synthetically generated oligonucleotide
gacatccagatgacccagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccccgacaggttcagtggcagtggatcaggcacagattttacactgaaaatcagcagagtggaggctgaggatgttggggtttattactgcatgcaagctctacaaactcctaggactttcggcggagggaccaaggtggagatcaaa

SEQ ID NO: 71 - protein, 112 aa, Artificial Sequence
  Synthetically generated peptide
DIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGGGTKVEIK

SEQ ID NO: 72 - DNA, 342 bp, Artificial Sequence
  Synthetically generated oligonucleotide
gaagttcaattgttagagtctggtggcggtcttgttcagcctggtggttctttacgtctttcttgcgctgcttccggattcactttctctcattactctatgatgtgggttcgccaagctcctggtaaaggtttggagtgggtttcttctatctttcctggtggctggactctttatgctgactccgttaaaggtcgcttcactatctctagagacaactctaagaatactctctacttgcagatgaacagcttaagggctgaggacactgcagtctactattgtgcgagagatcgggcagctgcctactggggccagggaaccctggtcaccgtctcaagc

SEQ ID NO: 73 - protein, 114 aa, Artificial Sequence
  Synthetically generated peptide
EVQLLESGGGLVQPGGSLRLSCAASGFTFSHYSMMWVRQAPGKGLEWVSSIFPGGWTLYADSVKGRFTISRDNSKNTLYLQMNSLRAEDTAVYYCARDRAAAYWGQGTLVTVSS

SEQ ID NO: 74 - DNA, 336 bp, Artificial Sequence
  Synthetically generated oligonucleotide
gacatccagatgacccagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccctggacgttcggccaagggaccaaggtggaaatcaaa

SEQ ID NO: 75 - protein, 112 aa, Artificial Sequence
  Synthetically generated peptide
DIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIK

SEQ ID NO: 76 - DNA, 351 bp, Artificial Sequence
  Synthetically generated oligonucleotide
gaagttcaattgttagagtctggtggcggtcttgttcagcctggtggttctttacgtctttcttgcgctgcttccggattcactttctctaattacactatgaattgggttcgccaagctcctggtaaaggtttggagtgggtttcttctatcgtttcttctggtggctttactaagtatgctgactccgttaaaggtcgcttcactatctctagagacaactctaagaatactctctacttgcagatgaacagcttaagggctgaggacactgcagtctactattgtgcgagaggctggtctagtcagcccgccatctggggccagggaagcctggtcaccgtctcaagc

SEQ ID NO: 77 - protein, 117 aa, Artificial Sequence
  Synthetically generated peptide
EVQLLESGGGLVQPGGSLRLSCAASGFTFSNYTMNWVRQAPGKGLEWVSSIVSSGGFTKYADSVKGRFTISRDNSKNTLYLQMNSLRAEDTAVYYCARGWSSQPAIWGQGSLVTVSS

SEQ ID NO: 78 - DNA, 321 bp, Artificial Sequence
  Synthetically generated oligonucleotide
gacatccagatgacccagtctccatcctccctgtctgcatctgtaggagacagagtcaccatcacttgccgggcaagtcagagcattggcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttactctacccctcggactttcggccctgggaccaaagtggatatcaaa

SEQ ID NO: 79 - protein, 107 aa, Artificial Sequence
  Synthetically generated peptide
DIQMTQSPSSLSASVGDRVTITCRASQSIGSYLNWYQQKPGKAPKLLIYAASSLQSGVPSRFSGSGSGTDFTLTISSLQPEDFATYYCQQSYSTPRTFGPGTKVDIK

SEQ ID NO: 80 - DNA, 366 bp, Artificial Sequence
  Synthetically generated oligonucleotide
gaagttcaattgttagagtctggtggcggtcttgttcagcctggtggttctttacgtctttcttgcgctgcttccggattcactttctcttggtactctatgtcttgggttcgccaagctcctggtaaaggtttggagtgggtttcttctatcggtccttctggtggccagactcgttatgctgactccgttaaaggtcgcttcactatctctagagacaactctaagaatactctctacttgcagatgaacagcttaagggctgaggacactgcagtctactattgtgcgagagattactatgatagtagtggttattcgtactttgactactggggccagggaacccaggtcaccgtctcaagc

SEQ ID NO: 81 - protein, 122 aa, Artificial Sequence
  Synthetically generated peptide
EVQLLESGGGLVQPGGSLRLSCAASGFTFSWYSMSWVRQAPGKGLEWVSSIGPSGGQTRYADSVKGRFTISRDNSKNTLYLQMNSLRAEDTAVYYCARDYYDSSGYSYFDYWGQGTQVTVSS

SEQ ID NO: 82 - DNA, 321 bp, Artificial Sequence
  Synthetically generated oligonucleotide
gacatccagatgacccagtctccactctccctgtctgcatctgtgggagacagagtcaccatcacttgtcgggcaagtcagagcattagcagccatttaaattggtatcagcggagaccagggaaagcccctaagctcctgatttatgctgcatccagtttgcaaagcggggtcccatcaaggttcagtggcagtggatctgggacagatttcgctctcaccatcagcagtctacaacctgaagattttgcagcttacttctgtcaccagagttccagtacgcctccgactttcggccaagggaccacggtggaaatcaaa

SEQ ID NO: 83 - protein, 107 aa, Artificial Sequence
  Synthetically generated peptide
DIQMTQSPLSLSASVGDRVTITCRASQSISSHLNWYQRRPGKAPKLLIYAASSLQSGVPSRFSGSGSGTDFALTISSLQPEDFAAYFCHQSSSTPPTFGQGTTVEIK

SEQ ID NO: 84 - DNA, 360 bp, Artificial Sequence
  Synthetically generated oligonucleotide
gaagttcaattgttagagtctggtggcggtcttgttcagcctggtggttctttacgtctttcttgcgctgcttccggattcactttctctccttacggtatggattgggttcgccaagctcctggtaaaggtttggagtgggtttcttctatctctccttctggtggcactactctttatgctgactccgttaaaggtcgcttcactatctctagagacaactctaagaatactctctacttgcagatgaacagcttaagggctgaggacactgcagtctactattgtgcgagacaaaaaaggtcctcgttaggtgcttttgatatctggggccaagggacaatggtcaccgtctcaagc

SEQ ID NO: 85 - protein, 120 aa, Artificial Sequence
  Synthetically generated peptide
EVQLLESGGGLVQPGGSLRLSCAASGFTFSPYGMDWVRQAPGKGLEWVSSISPSGGTTLYADSVKGRFTISRDNSKNTLYLQMNSLRAEDTAVYYCARQKRSSLGAFDIWGQGTMVTVSS

SEQ ID NO: 86 - DNA, 319 bp, Artificial Sequence
  Synthetically generated oligonucleotide
gactcagcctgcctccgtgtctgggtctcctggacagtcgatcaccatctcctgcactggaaccagcagtgacgttggtggttatagctatgtctcctggtaccaacagcacccaggcaaagcccccaaactcatgatttatgaggtcagtaatcggccctctggggtttctaatcgcttctctggctccaagtctggcaacacggcctccctgaccatctctgggctccaggctgaagacgaggctgattattactgcaactcatatacaagcagcagcactaagatgttcggcggagggaccaggctgaccgtccta

SEQ ID NO: 87 - protein, 110 aa, Artificial Sequence
  Synthetically generated peptide
QSVLTQPASVSGSPGQSITISCTGTSSDVGGYSYVSWYQQHPGKAPKLMIYEVSNRPSGVSNRFSGSKSGNTASLTISGLQAEDEADYYCNSYTSSSTKMFGGGTRLTVL

SEQ ID NO: 88 - DNA, 348 bp, Artificial Sequence
  Synthetically generated oligonucleotide
gaagttcaattgttagagtctggtggcggtcttgttcagcctggtggttctttacgtctttcttgcgctgcttccggattcactttctctaagtactctatggagtgggttcgccaagctcctggtaaaggtttggagtgggtttctcgtatctatccttctggtggccctactctttatgctgactccgttaaaggtcgcttcactatctctagagacaactctaagaatactctctacttgcagatgaacagcttaagggctgaggacactgcagtctactattgtgcgagagactcttacggcatggacgtctggggccaagggaccacggtcaccgtctcaagc

SEQ ID NO: 89 - protein, 116 aa, Artificial Sequence
  Synthetically generated peptide
EVQLLESGGGLVQPGGSLRLSCAASGFTFSKYSMEWVRQAPGKGLEWVSRIYPSGGPTLYADSVKGRFTISRDNSKNTLYLQMNSLRAEDTAVYYCARDSYGMDVWGQGTTVTVSS

SEQ ID NO: 90 - DNA, 333 bp, Artificial Sequence
  Synthetically generated oligonucleotide
gacatccagatgacccagtctccatcctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcaacagagtggaggctgaggatgttggggtttattactgcatgcaagctctacaaactccgacgttcggccaagggaccaaggtggaaatcaaa

SEQ ID NO: 91 - protein, 111 aa, Artificial Sequence
  Synthetically generated oligonucleotide
DIQMTQSPSSLPVTPGEPASISCRSSQSLLHSNGYNYLDWYLQKPGQSPQLLIYLGSNRASGVPDRFSGSGSGTDFTLKINRVEAEDVGVYYCMQALQTPTFGQGTKVEIK

SEQ ID NO: 92 - DNA, 348 bp, Artificial Sequence
  Synthetically generated oligonucleotide
gaagttcaattgttagagtctggtggcggtcttgttcagcctggtggttctttacgtctttcttgcgctgcttccggattcactttctcttattacggtatgggttgggttcgccaagctcctggtaaaggtttggagtgggtttcttctatcggtccttctggtggccttactaattatgctgactccgttaaaggtcgcttcactatctctagagacaactctaagaatactctctacttgcagatgaacagcttaagggctgaggacactgcagtctactattgtgcgagaggcactcgtacagtaaccaactggggccagggaaccctggtcaccgtctcaagc

SEQ ID NO: 93 - protein, 116 aa, Artificial Sequence
  Synthetically generated peptide
EVQLLESGGGLVQPGGSLRLSCAASGFTFSYYGMGWVRQAPGKGLEWVSSIGPSGGLTNYADSVKGRFTISRDNSKNTLYLQMNSLRAEDTAVYYCARGTRTVTNWGQGTLVTVSS

SEQ ID NO: 94 - DNA, 333 bp, Artificial Sequence; identical to SEQ ID NO: 12
  Synthetically generated oligonucleotide

SEQ ID NO: 95 - protein, 111 aa, Artificial Sequence; identical to SEQ ID NO: 13
  Synthetically generated peptide

SEQ ID NO: 96 - DNA, 369 bp, Artificial Sequence
  Synthetically generated oligonucleotide
gaagttcaattgttagagtctggtggcggtcttgttcagcctggtggttctttacgtctttcttgcgctgcttccggattcactttctctccttaccttatgtcttgggttcgccaagctcctggtaaaggtttggagtgggtttcttctatctattcttctggtggccttactgattatgctgactccgttaaaggtcgcttcactatctctagagacaactctaagaatactctctacttgcagatgaacagcttaagggctgaggacactgcagtctactattgtgcgagagacggttactatgatagtagtggttacgagggttttgactactggggccagggaaccctggtcaccgtctcaagc

SEQ ID NO: 97 - protein, 123 aa, Artificial Sequence
  Synthetically generated peptide
EVQLLESGGGLVQPGGSLRLSCAASGFTFSPYLMSWVRQAPGKGLEWVSSIYSSGGLTDYADSVKGRFTISRDNSKNTLYLQMNSLRAEDTAVYYCARDGYYDSSGYEGFDYWGQGTLVTVSS

SEQ ID NO: 98 - protein, 5 aa, Artificial Sequence
  Exemplary motif
XYXMX

SEQ ID NO: 99 - protein, 17 aa, Artificial Sequence
  Exemplary motif
SIXXSGGXTXYADSVKG

SEQ ID NO: 100 - protein, 8 aa, Artificial Sequence
  Exemplary sequence
DVGVGAAD

SEQ ID NO: 101 - protein, 13 aa, Artificial Sequence
  Exemplary sequence
DGYYDSSGYEGFD

SEQ ID NO: 102 - protein, 8 aa, Artificial Sequence
  Exemplary sequence
RSGSYPAD

SEQ ID NO: 103 - protein, 5 aa, Artificial Sequence
  Exemplary sequence
DRAAA

SEQ ID NO: 104 - protein, 7 aa, Artificial Sequence
  Exemplary sequence
GWSSQPA

SEQ ID NO: 105 - protein, 12 aa, Artificial Sequence
  Exemplary sequence
DYYDSSGYSYFD

SEQ ID NO: 106 - protein, 10 aa, Artificial Sequence
  Exemplary sequence
QKRSSLGAFD

SEQ ID NO: 107 - protein, 6 aa, Artificial Sequence
  Exemplary sequence
DSYGMD

SEQ ID NO: 108 - protein, 6 aa, Artificial Sequence
  Exemplary sequence
GTRTVT

SEQ ID NO: 109 - protein, 11 aa, Artificial Sequence
  Exemplary motif
RASQSIXSXLN

SEQ ID NO: 110 - protein, 6 aa, Artificial Sequence
  Exemplary sequence
ASSLQS

SEQ ID NO: 111 - protein, 9 aa, Artificial Sequence
  Exemplary motif
XQSXSTPXT

SEQ ID NO: 112 - protein, 14 aa, Artificial Sequence
  Synthetically generated sequence
TGTSSDVGGYSYVS

SEQ ID NO: 113 - protein, 6 aa, Artificial Sequence
  Synthetically generated sequence
EVSNRP

SEQ ID NO: 114 - protein, 10 aa, Artificial Sequence
  Synthetically generated sequence
NSYTSSSTKM

SEQ ID NO: 115 - protein, 5 aa, Artificial Sequence
  Exemplary sequence
LYRMR

SEQ ID NO: 116 - protein, 5 aa, Artificial Sequence
  Exemplary sequence
PYLMS

SEQ ID NO: 117 - protein, 5 aa, Artificial Sequence
  Exemplary sequence
EYGMG

SEQ ID NO: 118 - protein, 17 aa, Artificial Sequence
  Exemplary motif
SIXXSGGXTXYADSVKG

SEQ ID NO: 119 - protein, 17 aa, Artificial Sequence
  Exemplary sequence
SISPSGGITEYADSVKG

SEQ ID NO: 120 - protein, 17 aa, Artificial Sequence
  Exemplary sequence
SIYSSGGLTDYADSVKG

SEQ ID NO: 121 - protein, 17 aa, Artificial Sequence
  Exemplary sequence
SIVSSGGFTFYADSVKG

SEQ ID NO: 122 - protein, 16 aa, Artificial Sequence
  Synhetically generated peptide
RSSQSLLHSNGYNYLD

SEQ ID NO: 123 - protein, 7 aa, Artificial Sequence
  Synhetically generated peptide
LGSNRAS

SEQ ID NO: 124 - protein, 9 aa, Artificial Sequence
  Exemplary motif
MQALQXPXT

SEQ ID NO: 125 - protein, 8 aa, Artificial Sequence
  Exemplary sequence
MQALQPYT

SEQ ID NO: 126 - protein, 9 aa, Artificial Sequence
  Exemplary sequence
MQALQTPWT

SEQ ID NO: 127 - protein, 9 aa, Artificial Sequence
  Exemplary sequence
MQALQTPPT

SEQ ID NO: 128 - protein, 23 aa, Artificial Sequence
  Exemplary motif
DIQMTQSPXSLXXXXGXXXXIXC

SEQ ID NO: 129 - protein, 23 aa, Artificial Sequence
  Exemplary motif
DIQMTQSPXSLPXXXGXXXXIXC

SEQ ID NO: 130 - protein, 23 aa, Artificial Sequence
  Exemplary motif
DIQMTQSPXSLPVTPGXPASISC

SEQ ID NO: 131 - protein, 15 aa, Artificial Sequence
  Exemplary motif
WYXXXPGXXPXLLIY

SEQ ID NO: 132 - protein, 15 aa, Artificial Sequence
  Synthetically generated peptide
WYLQKPGQSPQLLIY

SEQ ID NO: 133 - protein, 15 aa, Artificial Sequence
  Synthetically generated peptide
WYQRRPGKAPKLLIY

SEQ ID NO: 134 - protein, 15 aa, Artificial Sequence
  Synthetically generated peptide
GVPXRFSGSGSGTDF

SEQ ID NO: 135 - protein, 32 aa, Artificial Sequence; identical to SEQ ID NO: 49
  Synthetically generated peptide

SEQ ID NO: 136 - protein, 32 aa, Artificial Sequence
  Synthetically generated peptide
GVPXRFSGSGSGTDFXLXIXXXXXEDXXXYXC

SEQ ID NO: 137 - protein, 10 aa, Artificial Sequence
  Synthetically generated peptide
FGXGTXXXIK

SEQ ID NO: 138 - protein, 10 aa, Artificial Sequence
  Synthetically generated peptide
FGXGTKXEIK

SEQ ID NO: 139 - protein, 30 aa, Artificial Sequence; identical to SEQ ID NO: 26
  Synthetically generated peptide

SEQ ID NO: 140 - protein, 14 aa, Artificial Sequence
  Synthetically generated peptide
WVRQAPGKGLEWVS

SEQ ID NO: 141 - protein, 32 aa, Artificial Sequence
  Synthetically generated peptide
RFTISRDNSKNTLYLQMNSLRAEDTAVYXCAX

SEQ ID NO: 142 - protein, 11 aa, Artificial Sequence
  Synthetically generated peptide
XWGQGXLVTVS

SEQ ID NO: 143 - protein, 12 aa, Artificial Sequence
  Synthetically generated peptide
YWGQGTLVTVSS

SEQ ID NO: 144 - protein, 238 aa, Artificial Sequence
  Synthetically generated peptide
MGWSCIILFLVATATGVHSDIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 238 aa, Artificial Sequence
  Synthetically generated peptide
MGWSCIILFLVATATGVHSDIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 237 aa, Artificial Sequence
  Synthetically generated peptide
MGWSCIILFLVATATGVHSDIQMTQSPLSLPVTPGGPASISCRSSQSLLHSNGYNYLDWYLQKPGQSPQLLIYLGSNRASGVPDRFSGSGSGTDFTLKISRVEAEDVGVYYCMQALQPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 238 aa, Artificial Sequence
  Synthetically generated peptide
MGWSCIILFLVATATGVHSDIQMTQSPLSLPVTPGEPASISCRSSQSLLHSNGYNYLDWYLQKPGQSPQLLIYLGSNRASGVPDRFSGSGSGTDFTLKISGVEAEDVGVYYCMQALQTGYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 238 aa, Artificial Sequence
  Synthetically generated peptide
MGWSCIILFLVATATGVHSDIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 238 aa, Artificial Sequence
  Synthetically generated peptide
MGWSCIILFLVATATGVHSDIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 233 aa, Artificial Sequence
  Synthetically generated peptide
MGWSCIILFLVATATGVHSDIQMTQSPSSLSASVGDRVTITCRASQSIGSYLNWYQQKPGKAPKLLIYAASSLQSGVPSRFSGSGSGTDFTLTISSLQPEDFATYYCQQSYSTPRTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 233 aa, Artificial Sequence
  Synthetically generated peptide
MGWSCIILFLVATATGVHSDIQMTQSPLSLSASVGDRVTITCRASQSISSHLNWYQRRPGKAPKLLIYAASSLQSGVPSRFSGSGSGTDFALTISSLQPEDFAAYFCHQSSSTPPTFGQGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 237 aa, Artificial Sequence
  Synthetically generated peptide
MGWSCIILFLVATATGVHSDIQMTQSPSSLPVTPGEPASISCRSSQSLLHSNGYNYLDWYLQKPGQSPQLLIYLGSNRASGVPDRFSGSGSGTDFTLKINRVEAEDVGVYYCMQALQTPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 237 aa, Artificial Sequence; identical to SEQ ID NO: 146
  Synthetically generated peptide

SEQ ID NO: 154 - protein, 235 aa, Artificial Sequence
  Synthetically generated peptide
MGWSCIILFLVATATGVHSQSVLTQPASVSGSPGQSITISCTGTSSDVGGYSYVSWYQQHPGKAPKLMIYEVSNRPSGVSNRFSGSKSGNTASLTISGLQAEDEADYYCNSYTSSSTKMFGGGTRLTVLGQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPAECS

SEQ ID NO: 155 - protein, 462 aa, Artificial Sequence
  Synthetically generated peptide
MGWSCIILFLVATATGAHSEVQLLESGGGLVQPGGSLRLSCAASGFTFSEYGMGWVRQAPGKGLEWVSSIVSSGGFTFYADSVKGRFTISRDNSKNTLYLQMNSLRAEDTAVYYCARGTRTVTNWGQGALVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - protein, 464 aa, Artificial Sequence
  Synthetically generated peptide
MGWSCIILFLVATATGAHSEVQLLESGGGLVQPGGSLRLSCAASGFTFSLYRMRWVRQAPGKGLEWVSSISPSGGITEYADSVKGRFTISRDNSKNTLYLQMNSLRAEDTAVYYCALDVGVGAADYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - protein, 469 aa, Artificial Sequence
  Synthetically generated peptide
MGWSCIILFLVATATGAHSEVQLLESGGGLVQPGGSLRLSCAASGFTFSPYLMSWVRQAPGKGLEWVSSIYSSGGLTDYADSVKGRFTISRDNSKNTLYLQMNSLRAEDTAVYHCARDGYYDSSGYEGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 464 aa, Artificial Sequence
  Synthetically generated peptide
MGWSCIILFLVATATGAHSEVQLLESGGGLVQPGGSLRLSCAASGFTFSKYTMWWVRQAPGKGLEWVSSIWSSGGFTRYADSVKGRFTISRDNSKNTLYLQMNSLRAEDTAVYYCAGRSGSYPADIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 460 aa, Artificial Sequence
  Synthetically generated peptide
MGWSCIILFLVATATGAHSEVQLLESGGGLVQPGGSLRLSCAASGFTFSHYSMMWVRQAPGKGLEWVSSIFPGGWTLYADSVKGRFTISRDNSKNTLYLQMNSLRAEDTAVYYCARDRAAAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - protein, 463 aa, Artificial Sequence
  Synthetically generated peptide
MGWSCIILFLVATATGAHSEVQLLESGGGLVQPGGSLRLSCAASGFTFSNYTMNWVRQAPGKGLEWVSSIVSSGGFTKYADSVKGRFTISRDNSKNTLYLQMNSLRAEDTAVYYCARGWSSQPAIWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 468 aa, Artificial Sequence
  Synthetically generated peptide
MGWSCIILFLVATATGAHSEVQLLESGGGLVQPGGSLRLSCAASGFTFSWYSMSWVRQAPGKGLEWVSSIGPSGGQTRYADSVKGRFTISRDNSKNTLYLQMNSLRAEDTAVYYCARDYYDSSGYSYFDYWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 466 aa, Artificial Sequence
  Synthetically generated peptide
MGWSCIILFLVATATGAHSEVQLLESGGGLVQPGGSLRLSCAASGFTFSPYGMDWVRQAPGKGLEWVSSISPSGGTTLYADSVKGRFTISRDNSKNTLYLQMNSLRAEDTAVYYCARQKRSSLGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 462 aa, Artificial Sequence
  Synthetically generated peptide
MGWSCIILFLVATATGAHSEVQLLESGGGLVQPGGSLRLSCAASGFTFSYYGMGWVRQAPGKGLEWVSSIGPSGGLTNYADSVKGRFTISRDNSKNTLYLQMNSLRAEDTAVYYCARGTRTVTNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 164 - protein, 469 aa, Artificial Sequence
  Synthetically generated peptide
MGWSCIILFLVATATGAHSEVQLLESGGGLVQPGGSLRLSCAASGFTFSPYLMSWVRQAPGKGLEWVSSIYSSGGLTDYADSVKGRFTISRDNSKNTLYLQMNSLRAEDTAVYYCARDGYYDSSGYEGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 165 - protein, 462 aa, Artificial Sequence
  Synthetically generated peptide
MGWSCIILFLVATATGAHSEVQLLESGGGLVQPGGSLRLSCAASGFTFSKYSMEWVRQAPGKGLEWVSRIYPSGGPTLYADSVKGRFTISRDNSKNTLYLQMNSLRAEDTAVYYCARDSYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2004/0110933 A1
(43) Pub. Date: Jun. 10, 2004

(54) CD44-binding ligands

(75) Inventors: Isaac J. Rondon, San Francisco, CA (US); Albert Edge, Newton, MA (US); Rachel Baribault Kent, Boxborough, MA (US)

Correspondence Address: FISH & RICHARDSON PC, 225 FRANKLIN ST, BOSTON, MA, 02110, US

(73) Assignee: DYAX CORPORATION

(21) Appl. No.: 10/663,244

(22) Filed: Sep. 15, 2003

Related U.S. Application Data
(60) Provisional application No. 60/410,758, filed on Sep. 13, 2002.
(60) Provisional application No. 60/469,123, filed on May 9, 2003.

(51) Int. Cl. C07K 16/28

(52) U.S. Cl. 530/388.22

(57) ABSTRACT

The invention provides, inter alia, CD44-binding proteins, including CD4-binding antibodies, antibody fragments, and pharmaceutical compositions thereof, as well as nucleic acids, recombinant expression vectors and host cells for making such proteins. Methods of using the proteins to detect CD44 or to modulate a CD44-expressing cell, e.g., in a subject, are also described.

### CROSS-REFERENCE TO RELATED APPLICATIONS

This application claims priority to U.S. Provisional Patent Applications Serial No. 60/410,758, filed on Sep. 13, 2002, and 60/469,123, filed May 9, 2003, the contents of which are hereby incorporated by reference in their entireties.

### BACKGROUND

CD44 is a highly conserved gene found in mammals. Goodison et al. (1999) Mol. Pathol. 52(4):189-96. It encodes a type I transmembrane protein and is a member of the cartilage link protein family. Bajorath (2000) Proteins 39(2):103-11. Through alternative splicing, the CD44 gene gives rise to many different CD44 protein isoforms, which tend to be expressed in a cell-specific manner and differentially glycosylated. CD44 is expressed in many different tissues, including white blood cells and metastatic cancer cells, where it functions in cell-cell and cell-matrix adhesion, as well as in signal transduction. A major endogenous ligand of CD44 is hyaluronic acid (HA), an integral component of the extracellular matrix. Other endogenous CD44 ligands include: osteopontin, serglycin, collagen, fibronectin, and laminin.

Adhesive interactions between receptors on vascular endothelial cells (ECs) and circulating leukocytes regulate the extravasation of leukocytes at sites of inflammation. Activated CD44 can bind to hyaluronic acid (HA). The affinity of CD44 present on the surface of leukocytes for its ligand HA is subject to regulation. In resting leukocytes, the affinity of CD44 for HA is relatively low (about 150 μM for murine CD44). Upon T cell receptor activation, CD44 becomes activated resulting in an increase in its affinity for HA (to about 5 μM for murine CD44). The interaction between activated CD44 and HA can mediate the extravasation of activated leukocytes into an inflamed site. CD44 can mediate the adhesion and migration of metastatic tumor cells. See generally, e.g., Isacke and Yarwood (2002) Int. J. Biochem. Cell Biol. 34(7):718-21; Siegelman et al. (1999) J Leukoc. Biol. 66(2):315-21; Pure and Cuff (2001) Trends Mol Med 7(5):213-21; and Yasuda et al. (2002) Histol Histopathol 17(3):945-50.

### SUMMARY

The invention provides, inter alia, CD44-binding antibodies, antibody fragments, and pharmaceutical compositions thereof, as well as nucleic acids, recombinant expression vectors and host cells for making such antibodies and fragments. Methods of using the antibodies to detect CD44 or to modulate a CD44-expressing cell, e.g., in a subject, are also described.

In one aspect, the invention features a protein that interacts with, e.g., binds to CD44, e.g., human CD44, with high affinity and specificity. For example, the protein binds to human CD44 with an affinity constant of at least 2×10⁷ M⁻¹, e.g., at least 10⁸ M⁻¹, 10⁹ M⁻¹, or 10¹⁰ M⁻¹. In one embodiment, the protein includes one or more human CDRs, e.g., one, two, three, four, five, or six human CDRs. For example, the LC CDRs can be human. In another example, HC CDR3 is human.

In one embodiment, the protein binds to activated CD44 with an affinity (Ka) higher (e.g., 1.2, 1.5, 1.8, 2, 3, 4, 5, 10, 20, 50, 100-fold higher) than their affinity for resting CD44. In one embodiment, the protein binds to deglycosylated CD44 (e.g., CD44 antigen produced in an activated lymphocyte or CD44 antigen that has been treated with a glycosidase enzyme, e.g., deglycosylated CD44Fc) with an affinity (Ka) higher (e.g., 1.2, 1.5, 1.8, 2, 3, 4, 5, 10, 20, 50, 100-fold higher) than their affinity for resting CD44. In still another embodiment, the protein binds to high-affinity CD44 (i.e., CD44 that displays an increase in affinity for HA as compared to resting CD44) with an affinity (Ka) higher (e.g., 1.2, 1.5, 1.8, 2, 3, 4, 5, 10, 20, 50, 100-fold higher) than their affinity for resting CD44.

In one embodiment, the protein interacts with, e.g., binds to, the extracellular domain of CD44, e.g., a hyaluronic acid (HA) binding domain or fragment of human CD44 (e.g., about amino acids 21-649, or about amino acids 21-222, of SEQ ID NO:1). In some embodiments, the protein binds to the extracellular domain of human CD44 (e.g., the HA binding domain) and inhibits the binding of CD44 to HA. For example, the protein inhibits HA binding and includes one or more features (e.g., CDRs) of HAE-A3, HAE-G2, or HAE-H10.

In one embodiment, the CD44-binding protein binds all or part of the epitope bound by a polypeptide or antibody described herein, e.g., BE-B 12, BE-D7, HAE-B8, HAE-F1, BE-A11, F2, HAE-H10, HAE-H-H10 (also referred to as HH10), H1, HAE-A3, BE-H10 (referred to as BH10), HAE-H9, or HAE-G2. For example, the CD44-binding protein can inhibit, e.g., competitively inhibit, the binding of a polypeptide or antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G2, to human CD44. The protein may bind to an epitope, e.g., a conformational or a linear epitope, which epitope when bound prevents binding of a polypeptide or antibody described herein, BE-B12, BE-D7, HAE-B8, HAE-F1, BE-A11, F2, HAE-H10, HAE-H-H10, H1, HAE-A3, BE-H10, HAE-H9, or HAE-G2. The epitope can be in close proximity spatially or functionally-associated, e.g., an overlapping or adjacent epitope in linear sequence or conformationally to the one recognized by the BE-B 12, BE-D7, HAE-B8, HAE-F1, BE-A11, F2, HAE-H10, HAE-H-H10, H1, HAE-A3, BE-H10, HAE-H9, or HAE-G2 antibody. In one embodiment, the CD44-binding protein binds to an epitope located wholly or partially within the region of about amino acids 21 to 649, 40-200, 120-200, 120-170, or 22-222 of SEQ ID NO:1. In one embodiment, the protein binds to an epitope that does not include an amino acid encoded by the v6 exon.

The protein can bind to a CD44-expressing cell. For example, Human CD44 is expressed on many different cell types, including white blood cells (e.g., B cells, T cells, macrophages, and neutrophils), parenchymal cells, and tumor cells (e.g., cancerous lung, liver, colon, breast, ovarian, epidermal, laryngeal, and cartilage cells, and particularly metastatic cells thereof). In some embodiments, the protein can be internalized with the CD44 by a living cell, thereby providing intracellular delivery of an agent conjugated to the antibody, e.g., a cytotoxic or a labeling agent.

In on embodiment, the protein is used to modulate the activity of a CD44-expressing cell, e.g., in a subject or in vitro. For example, the protein can be used to therapeutically target living normal, benign hyperplastic, and cancerous cells that express CD44 in a subject, e.g., a human subject. In one embodiments, the protein preferentially binds to CD44 expressed on activated lymphocytes or parenchymal cells (e.g., they bind with higher affinity (Ka), e.g., at least 1.2, 1.5, 1.8, 2, 3, 4, 5, 10, 20, 50, 100-fold, or more, higher, to activated lymphocytes than they bind to resting lymphocytes). In some related embodiments, the protein binds to neuraminidase treated KG1a cells with an affinity (Ka) at least 1.2, 1.5, 1.8, 2, 3, 4, 5, 10, 20, 50, 100-fold, or more, higher than their affinity for untreated KG1a cells. In one embodiment, the protein has an IC50 for inhibition of KG1a cells for binding to HA of less than 10⁻⁷, 10⁻⁸, 10⁻⁹, 10⁻¹⁰, or 10⁻¹¹ M. In another embodiments, the protein binds to CD44 present on activated lymphocytes and parenchymal cells and accumulate at sites of inflammation, e.g., in vivo. In another embodiment, the protein binds to CD44 on lymphocytes, e.g., activated lymphocytes, and thereby inhibit (e.g., partially or completely) lymphocyte rolling on endothelial cells, e.g., in vivo. In still another embodiments, the protein binds to CD44 present on living cells, e.g., white blood cells (e.g., activated lymphocytes) or cancer cells (e.g., metastatic cancer cells), and inhibit (e.g., partially or completely) migration and/or extravasation of such cells from endothelial vessels. In still another embodiments, the protein is a CD44 activity enhancing ligand, a CD44-binding cell agonist, e.g., a CD44-binding NK-cell agonist, or a CD44-binding cell sensitizing agent, e.g., a CD44-binding NK-cell sensitizing agent.

In one aspect, the invention features a protein that binds to CD44 ectodomain, e.g., with a K_{d} of less than 5×10⁻⁶ M or 2×10⁻⁷ M. The protein includes a heavy chain immunoglobulin variable domain and a light chain immunoglobulin variable domain. Such ligands are referred to as “CD44-binding antibodies” or “CD44-binding antibodies.”

In some embodiments, the protein includes one or more of the following features: (1) a HC CDR1 sequence motif that includes X₁—Y—X₂—M—X₃ (SEQ ID NO:98), wherein X₁ is any amino acid (e.g., E, L, K, H, N, W, or P), X₂ is any amino acid (e.g., G, R, S, T, or L), and X₃ is any amino acid (e.g., G, R, W, M, N, D, E, or S); (2) a HC CDR2 sequence that includes: X₁—I—X₂—X₃—X₄—G-G-X₅-T-X₆-Y-A-D-S—V—K-G (SEQ ID NO:99), where X₁ is any amino acid (e.g., S or R); X₂ is any amino acid (e.g., V, S, Y, W, F, V, G, or S), X₃ is any amino acid (e.g., S or P), X₄ is S or absent, X₅ is any amino acid (e.g., hydrophobic (e.g., F, I, L, W, or P) or uncharged polar (e.g., Q or T)), and X₆ is any amino acid (e.g., F, E, D, R, L, or K); and (3) a HC CDR3 sequence that includes one of the following exemplary sequences: DVGVGAAD (SEQ ID NO:100), DGYYDSSGYEGFD (SEQ ID NO:101), RSGSYPAD (SEQ ID NO:102), DRAAA (SEQ ID NO:103), GWSSQPA (SEQ ID NO:104), DYYDSSGYSYFD (SEQ ID NO:105), QKRSSLGAFD (SEQ ID NO:106), DSYGMD (SEQ ID NO:107), and GTRTVT (SEQ ID NO:108), or a sequence that is at least 85% identical to one of the foregoing.

In some embodiments, the protein includes one or more of the following features: (1) a LC CDR1 (e.g., a kappa LC) that includes R-A-S-Q-S—I₁—X₂-L-N (SEQ ID NO:109), wherein X₁is any amino acid (e.g., G or S) and X₂ is any amino acid (e.g., Y or H), or a sequence that differs by no more than two or one amino acid substitutions; (2) a LC CDR2 (e.g., a kappa LC) that includes an amino acid sequence of at least 6 amino acids of which at least 5 or 6 amino acids are identical to ASSLQS (SEQ ID NO:110); and (3) a LC CDR3 (e.g., a kappa LC) that includes X₁-Q-S—X₂—S-T-P—X₃-T (SEQ ID NO:111), where XI is any amino acid (e.g., hydrophilic, e.g., Q or H), and X₂ is any amino acid (e.g., Y or S), X₃ is any amino acid (e.g., R or P).

In some embodiments, the protein includes one or more of the following features: (1) a LC CDR1 (e.g., a lambda LC) that includes an amino acid sequence of at least 11, 12, or 14 amino acids of which at least 9, 10, 11, 12, 13, or 14 amino acids are identical to TGTSSDVGGYSYVS (SEQ ID NO:112); (2) a LC CDR2 (e.g., a lambda LC) that includes an amino acid sequence of at least 7 amino acids of which at least 5, 6 or 7 amino acids are identical to EVSNRP (SEQ ID NO:113); and (3) a LC CDR3 (e.g., a lambda LC) that includes an amino acid sequence of at least 9 or 10 amino acids of which at least 7, 8, 9, or 10 amino acids are identical to NSYTSSSTKM (SEQ ID NO:114).

In one embodiment, the protein includes a HC variable domain that includes a CDR₁ sequence motif of X₁—I—Y—X₂-M-X₃ (SEQ ID NO:98), wherein X₁, X₂, and X₃ are any amino acid, or X₁is E, L or P, X₂ is G, R, or L, and X₃ is G, R, or S; and/or one of the following exemplary sequences: LYRMR (SEQ ID NO:115), PYLMS (SEQ ID NO:116), and EYGMG (SEQ ID NO:117). Other exemplary CDR1 amino acid sequences have a length of at least 5 amino acids of which at least 3, 4, or 5 amino acids are identical to a HC CDR1 sequence described herein.

In one embodiment, the protein includes HC variable domain that includes a CDR2 sequence with the following motif: S—I—X₁—X₂—S-G-G-X₃-T-X₄—Y-A-D-S—V—K-G (SEQ ID NO:118), where X₁ is any amino acid (e.g., valine, serine, or tyrosine), X₂ is any amino acid (e.g., proline or serine), X₃ is hydrophobic (e.g., phenylalanine, isoleucine, leucine, valine, methionine, tryptophan, or tyrosine), and X₄ is any amino acid (e.g., phenylalanine, aspartic acid, glutamic acid, or acidic or aromatic). Exemplary sequences that match this motif include: SISPSGGITEYADSVKG (SEQ ID NO:119), SIYSSGGLTDYADSVKG (SEQ ID NO:120), and SIVSSGGFTFYADSVKG (SEQ ID NO:121). Other exemplary CDR2 amino acid sequences have an amino acid sequence of at least 15, 16, or 17 amino acids of which at least 10, 12, 14, 15, 16, or 17 amino acids are identical to a HC CDR2 sequence described herein.

In one embodiment, the protein includes a HC variable domain that includes a CDR3 sequence including one of the following exemplary sequences: DVGVGAAD (SEQ ID NO:100), DGYYDSSGYEGFD (SEQ ID NO:101), and GTRTVT (SEQ ID NO:108), and/or an amino acid sequence of at least 7 or 8 amino acids of which at least 5, 6, 7, or 8 amino acids are identical to DVGVGAAD (SEQ ID NO:100); and/or an amino acid sequence of at least 11, 12, or 13 amino acids of which at least 8, 9, 10, 11, 12, or 13 amino acids are identical to DGYYDSSGYEGFD (SEQ ID NO:101); and/or an amino acid sequence of at least 5 or 6 amino acids of which at least 3, 4, 5, or 6 amino acids are identical to GTRTVT (SEQ ID NO:108). In some embodiments, the CDR3 sequence is less than 15, 13, 11, 9 or 7 amino acids in length.

In one embodiment, the protein includes a LC variable domain that includes a CDR1 sequence which includes: the sequence RSSQSLLHSNGYNYLD (SEQ ID NO:122) ; and/or an amino acid sequence of at least 13, 15 or 16 amino acids of which at least 11, 13, 14, 15, or 16 amino acids are identical to RSSQSLLHSNGYNYLD (SEQ ID NO:122).

In one embodiment, the protein includes a LC variable domain that includes a CDR2 sequence which includes: the sequence LGSNRAS (SEQ ID NO:123); and/or an amino acid sequence of at least 4, 6 or 7 amino acids of which at least 3, 5, 6, or 7 amino acids are identical to LGSNRAS (SEQ ID NO:123).

In one embodiment, the protein includes a LC variable domain that includes a CDR3 sequence which includes: the motif M-Q-A-L-Q-X₁—P—X₂-T (SEQ ID NO:124), where X₁ is any amino acid, (e.g., threonine) or absent, and X₂ is any amino acid (e.g., hydrophobic, (e.g., tryptophan, proline or phenylalanine), tyrosine, or arginine) or absent; one of the following exemplary sequences: MQALQPYT (SEQ ID NO:125), MQALQTPWT (SEQ ID NO:126), or MQALQTPPT (SEQ ID NO:127); and/or an amino acid sequence of at least 6, 8 or 9 amino acids of which at least 5, 6, 7, 8, or 9 amino acids are identical to MQALQPYT (SEQ ID NO:125), MQALQTPWT (SEQ ID NO:126) or MQALQTPPT (SEQ ID NO:127).

In one embodiment, the CDR regions of the heavy or light chain variable domain (e.g., one or more of CDR1, CDR2, and CDR3) are at least 60, 70, 80, 90, 92, 95, 96, 97, 98, or 99% identical to a corresponding heavy or light CDR sequence described herein, e.g., a CDR in of the following antibodies: BE-B12, BE-D7, HAE-B8, HAE-F1, BE-A11, F2, HAE-H10, HAE-H-H10, H1, HAE-A3, BE-H10, HAE-H9, or HAE-G2.

In one embodiment, the framework regions of the heavy or light chain variable domain (e.g., one or more of FR1, FR2, FR3, and FR4) are at least 60, 70, 80, 90, 92, 95, 96, 97, 98, or 99% identical to a corresponding heavy or light FR sequence, e.g., a known sequence or a sequence described herein. For example, the framework region or the corresponding sequence to which the framework region is related is human.

In another embodiment, framework features can include one or more of the following features. For example, FR1-L can include D-I-Q-M-T-Q-S—P—X₁I-—-L-X₂—X₃—X₄—X₅-G-X₆—X₇—X₈—X₉—I—X₁₀—C (SEQ ID NO:128), wherein X₁ is L or S, X₂ is P or S, X₃ is a small amino acid (e.g., fewer than four side chain carbons, e.g., A, V, or G), X₄ is T or S, X₅ is V or P, X₆ is E, D, or G, X₇ is P or R, X₈ is A or V, X₉ is S or T, X₁₀ is S or T; D-I-Q-M-T-Q-S—P-X₁—S-L-P—X₂—X₃—X₄-G-X₅—X₆—X₇—X₈—I—X₉—C (SEQ ID NO:129), wherein X₁ is L or S, X₂ is a small amino acid (e.g., fewer than four side chain carbons, e.g., A, V, or G), X₃ is T or S, X₄ is V or P, X₄ is aliphatic (e.g., A, V, or I) is X₅ is E, D, or G, X₆ is P or R, X₇ is A or V, X₈ is S or T, X₉ is S or T; or a sequence which differs by at least one, but less than 6, 5, 3, or 2 amino acids from D-I-Q-M-T-Q-S—P—X₁—I—S-L-X₂—X₃—X₄—X₅-G-X₆—X₇—X₈—X₉—I—X₁₀—C (SEQ ID NO:128) at positions other than X (e.g., a substitution, e.g., a conservative substitution). FR1-L can include DIQMTQSPX₁SLPVTPGX₂PASISC (SEQ ID NO:130) w (e. g., leucine or serine), X₂ is any amino acid (e.g., glycine or glutamic acid), or a sequence which differs by at least one, but less than 7, 6, 5, 3, or 2 amino acids from DIQMTQSPX₁SLPVTPGX₂PASISC (SEQ ID NO:130) at positions other than X (e.g., a substitution, e.g., a conservative substitution).

FR2-L can include W-Y—X₁—X₂—X₃—P-G-X₄—X₅—P—X₆-L-L-I—Y (SEQ ID NO:131), wherein X₁ is L or Q, X₂ is Q or R, X₃ is K or R, X₄ is Q or K, X₅ is S or A, X₆ is Q or K, or a sequence which differs by at least one, but less than 5, 4, 3, or 2 amino acids from WYLQKPGQSPQLLIY (SEQ ID NO:132) or WYQRRPGKAPKLLIY (SEQ ID NO:133). FR2-L can include WYLQKPGQSPQLLIY (SEQ ID NO:132) or a sequence which differs by at least one, but less than 5, 4, 3, or 2 amino acids from SEQ ID NO:132 (e.g., a substitution, e.g., a conservative substitution).

FR3-L can include GVPXIRFSGSGSGTDF (SEQ ID NO:134), wherein X₁ is any amino acid (e.g., D or S), or a sequence which differs by at least one, but less than 3, or 2 amino acids from GVPX₁RFSGSGSGTDF (SEQ ID NO:134) at positions other than indicated by X (e.g., a substitution, e.g., a conservative substitution). FR3-L can include GVPDRFSGSGSGTDFFLKISRVEAEDVGVYYC (SEQ ID NO:135) or a sequence which differs by at least one, but less than 5, 4, 3, or 2 amino acids from GVPDRFSGSGSGTDFTLKISRVEAEDVGVYYC (SEQ ID NO:135) (e.g., a substitution, e.g., a conservative substitution) (e.g., a substitution, e.g., a conservative substitution).

FR3-L which comprises GVP—X₁—RFSGSGSGTDF—X₂-L-X₃—I—X₄—X₅—X₆—X₇—X₈-ED-X₉—X₁₀—X₁₁—I—Y—X₁₂—C (SEQ ID NO:136), wherein X₁ is any amino acid (e.g., D or S), X₂ is any amino acid (e.g., T or A), X₃ is any amino acid (e.g., K or T) X₄ is any amino acid (e.g., S or N) X₅ is any amino acid (e.g., R, G, or S) X₆ is any amino acid (e.g., V or L), X₇ is any amino acid (e.g., E or Q), X₈ is any amino acid (e.g., A or P), X₉ is any amino acid (e.g., V or F), X₁₀ is any amino acid (e.g., G or A), X₁₁ is any amino acid (V, T, or A), X₁₂ is aromatic, or a sequence which differs by at least one, but less than 3, or 2 amino acids from GVP—X₁—RFSGSGSGTDF—X₂-L-X₃—I—X₄—X₅—X₆—X₇—X₈-E-D-X₉—X₁₀—X₁₁—Y—X₁₂—C (SEQ ID NO:136) at positions other than indicated by X (e.g., a substitution, e.g., a conservative substitution). FR4-L can include F-G-X₁-G-T-X₂—X₃—X₄—I—K (SEQ ID NO:137), wherein X₁ is any amino acid (e.g., G, Q, or P), and X₂ is K, T, or R, X₃ is hydrophobic (e.g., aliphatic, e.g., V or L), X₄ is hydrophilic (e.g., E, D, or T), or a sequence which differs by at least one, but less than 3, or 2 amino acids from F-G-X₁-G-T-X₂—X₃—X₄—I—K (SEQ ID NO:137) at positions other than indicated by X (e.g., a substitution, e.g., a conservative substitution).

FR4-L can include FGX₁GTKX₂EIK (SEQ ID NO:138), wherein X₁ is glycine or glutamine, and X₂ is leucine or valine. FR4-L can include FGX₁GTKX₂EIK (SEQ ID NO:138), wherein X₁ is any amino acid (e.g., glycine or glutamine), and X₂ is hydrophobic (e.g., leucine or valine), or a sequence which differs by at least one, but less than 3, or 2 amino acids from FGX₁GTKX₂EIK (SEQ ID NO:138) at positions other than X₁ or X₂ (e.g., a substitution, e.g., a conservative substitution).

FR1-H can include EVQLLESGGGLVQPGGSLRLSCAASGFTFS (SEQ ID NO:139) or a sequence which differs by at least one, but less than 7, 6, 5, 3, or 2 amino acids from EVQLLESGGGLVQPGGSLRLSCAASGFTFS (SEQ ID NO:139)(e.g., a substitution, e.g., a conservative substitution).

FR2-H can include WVRQAPGKGLEWVS (SEQ ID NO:140), or a sequence which differs by at least one, but less than 5, 4, 3, or 2 amino acids from WVRQAPGKGLEWVS (SEQ ID NO:140)(e.g., a substitution, e.g., a conservative substitution).

FR3-H can include RFTISRDNSKNTLYLQMNSLRAEDTAVYX₁CAX₂ (SEQ ID NO:141)where X₁ can be any amino acid, e.g., tyrosine or histidine and X₂ can be any amino acid, e.g., arginine, glycine or leucine, or a sequence that differs from RFFISRDNSKNTLYLQMNSLRAEDTAVYX₁CAX₂ (SEQ ID NO:141) by at least one, but less than 5, 4, 3, or 2 amino acids (e.g., a substitution, e.g., a conservative substitution).

FR4-H can include X1WGQGX₂LVTVS (SEQ ID NO:142), wherein X₁ is any amino acid (e.g., asparagine or tyrosine), and X₂ is any amino acid (e.g., alanine or threonine), or a sequence that differs from YWGQGTLVTVSS (SEQ ID NO:143) by at least one, but less than 4, 3, or 2 amino acids (e.g., a substitution, e.g., a conservative substitution).

In one embodiment, a CD44-binding protein includes one or more of the following features: it (1) detectably binds to a CD44-expressing cell (e.g., as detected by a method described herein), at a concentration of less than 1000, 500, 200, 100, 50, 25, or 20 μg/ml; (2) inhibits HA binding to a CD44-expressing cell (e.g., CD44+KG1a cells) by at least 20, 40, 60, or 80% inhibition at a concentration of less than 500, 200, 100, 50, or 20 μg/ml (e.g., in vitro or in vivo); (3) inhibits leukocyte-endothelial cell adhesion (e.g., adherence between HMEC cells and KG1a cells); (4) affects cell migration (e.g., endothelial cells, e.g., after wound infliction); and/or (5) affects wound healing (e.g., in an in vitro assay or in vivo).

The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The CD44-binding antibodies can be full-length (e.g., an IgG (e.g., an IgG1, IgG2, IgG3, IgG4), IgM, IgA (e.g., IgA1, IgA2), IgD, and IgE) or can include only an antigen-binding fragment (e.g., a Fab, F(ab′)₂ or scFv fragment). The antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A CD44-binding antibody can include a heavy and light chain constant region substantially from a human antibody, e.g., a human IgG1 constant region or a portion thereof. As used herein, “isotype” refers to the antibody class (e.g., IgM or IgG1) that is encoded by heavy chain constant region genes.

In a preferred embodiment, the antibody (or fragment thereof) is a recombinant CD44-binding antibody or modified CD44-binding antibody. For example, the antibody is a chimeric, a humanized, a deimmunized, or an in vitro generated antibody. The term “recombinant” or “modified” human antibody, as used herein, is intended to include all antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, deimmunized, in vitro generated antibodies, and may optionally include constant regions derived from human germline immunoglobulin sequences.

In one embodiment, the CD44-binding antibody binds to an epitope distinct from an epitope bound by known CD44-binding antibodies, e.g., MAbs OS/37 (Cao et al., Immunobiology. 1995 193(1)1-14. and Murakami et al., J. Immunol. 1994 152(2)467-77.), BU52 (Guo et al., Int Immunol. 1994 6(2)213-21. and Guy et al., Immunology. 1992 75(4)713-6.), Hermes-3 (de los Toyos et al., Blood. 1989 74(2)751-60. and Picker et al., J Immunol. 1989 142(6)2046-51.), AIG3 and A3D8 (Haynes et al. (1983) J. Immunol. 131:1195-1200, Telen et al. (1983) J. Clin. Invest. 71; 1878-1886), and TL-1 (Cao et al., Immunobiology. 1996-97 196(5)504-12. and Cao et al., Immunobiology. 1995 193(1): 1-14.). In other embodiments, the CD44-binding antibody does not compete with known CD44-binding antibodies, e.g., MAbs OS/37, BU52, Hermes-3, and TL-1, for binding to CD44. In still other embodiments, the CD44-binding antibody does not compete with a CD44-binding ligand described herein, e.g., BE-B12, BE-D7, HAE-B8, HAE-F1, BE-Al1, F2, HAE-H10, HAE-H-H10, H1, HAE-A3, BE-H10, HAE-H9, or HAE-G2.

In one embodiment, the CD44-binding antibody binds to an epitope that overlaps with or is identical to an epitope bound by a known CD44-binding antibody, e.g., the S5 monoclonal antibody or the IM7 antibody.

In other embodiments, the CD44-binding antibody is a human antibody. In some embodiments, the antibody is produced by a cell that includes one or more nucleic acid molecules selected from the group consisting of nucleic acids encoding an immunoglobulin HC or LC of an antibody described herein, e.g., BE-B 12, BE-D7, HAE-B8, HAE-F1, BE-A11, F2, HAE-H10, HAE-H-H10, H1, HAE-A3, BE-H10, HAE-H9, or HAE-G2; a nucleic acid that differs from one of these sequences by 5, 10, 15, 20, or 25 bases or less (e.g., by a substitution (e.g., a silent codon change), non-frameshifting insertion, or non-frameshifting deletion), a nucleic acid that hybridizes under high stringency conditions to such a nucleic acid sequence; or a nucleic acid that encodes a polypeptide that includes the amino acid sequence of an immunoglobulin HC or LC of an antibody described herein, e.g., BE-B12, BE-D7, HAE-B8, HAE-F1, BE-A11, F2, HAE-H10, HAE-H-H10, H1, HAE-A3, BE-H10, HAE-H9, or a nucleic acid or polypeptide described herein (e.g., a homologous nucleic acid or polypeptide described herein), or a polypeptide that differs from one of these by 1, 2, 3, 5, 7, 10, 12, 15, or 20 amino acids or less (e.g., by a substitution (e.g., a conservative substitution), insertion, or deletion).

Also within the scope of the invention are antibodies, or antigen-binding fragments thereof, which bind overlapping epitopes of, or competitively inhibit, the binding of the CD44-binding antibodies disclosed herein to CD44, e.g., antibodies which bind overlapping epitopes of, or competitively inhibit, the binding of monospecific antibodies, e.g., BE-B 12, BE-D7, HAE-B8, HAE-F1, BE-A11, F2, HAE-H10, HAE-H-H10, H1, HAE-A3, BE-H10, HAE-H9, or HAE-G2, to CD44. Any combination of CD44-binding antibodies is within the scope of the invention, e.g., two or more antibodies that bind to different regions of CD44, e.g., antibodies that bind to two different epitopes on the extracellular domain of CD44, e.g., a bispecific antibody.

In one embodiment, the CD44-binding antibody, or antigen-binding fragment thereof, includes at least one light or heavy chain immunoglobulin (or preferably, at least one light chain immunoglobulin and at least one heavy chain immunoglobulin). Preferably, each immunoglobulin includes a light or a heavy chain variable region having at least one, two and, preferably, three complementarity determining regions (CDR's) substantially identical to a CDR from a CD44-binding light or heavy chain variable region, respectively, e.g., from a light chain variable (VLC) region or a heavy chain variable (VHC) region from BE-B12, BE-D7, HAE-B8, HAE-F1, BE-A11, F2, HAE-H10, HAE-H-H10, H1, HAE-A3, BE-H10, HAE-H9, or HAE-G2 The light chain and heavy chain variable regions of such regions are shown in Table 1.

TABLE 1

| | | |
|---|---|---|
| Antibody | Sequence | Identifier |

| | | |
|---|---|---|
| F2 VLC | GACATCCAGATGACCCAGTCTCCACTCTCCCTGGCCGT | SEQ ID NO:4 |
| Nucleic | CACCCTTGGACAGCCGGCCTCCATCTCCTGCAGGTCTA | |
| Acid | GTGAAAGCCTCGTGTACAGTGATGGAAACACCTACTTG | |
| Sequence | GGTTGGTTTCAGCAGAGGCCAGGCCAATCTCCACGGCG | |
| | CCTACTTTATAAGGTTTCTAACCGGGACTCTGGGGTCC | |
| | CAGACAGATTCAGCGGCAGTGGGTCAGGCACTGATTTC | |
| | ACACTGCACATCAGCAGGGTGGAGGCTGAAGATGTTGG | |
| | GGTTTATTACTGCATGCATTCTATACGCTGGCCGTGGA | |
| | CGTTCGGCCAAGGGACCACGGTGGAAATCAAG | |
| F2 VLC | DIQMTQSPLSLAVTLGQPASISCRSSESLVYSDGNTYL | SEQ ID NO:5 |
| Amino | GWFQQRPGQSPRRLLYKVSNRDSGVPDRFSGSGSGTDF | |
| Acid | TLHISRVEAEDVGVYYCMHSIRWPWTFGQGTTVEIK | |
| Sequence | | |
| F2 VHC | GAAGTTCAATTGTTAGAGTCTGGTGGCGGTCTTGTTCA | SEQ ID NO:6 |
| Nucleic | GCCTGGTGGTTCTTTACGTCTTTCTTGCGCTGCTTCCG | |
| Acid | GATTCACTTTCTCTCCTTACACTATGGCTTGGGTTCGC | |
| Sequence | CAAGCTCCTGGTAAAGGTTTGGAGTGGGTTTCTTCTAT | |
| | CTATCCTTCTGGTGGCACTACTCCTTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGACATTTTACTG | |
| | TGTATGATGGTTTTGATTTGTGGGGCCGAGGGACAATG | |
| | GTCACCGTCTCAAGC | |
| F2 VHC | EVQLLESGGGLVQPGGSLRLSCAASGFTFSPYTMAWVR | SEQ ID NO:7 |
| Amino | QAPGKGLEWVSSIYPSGGTTPYADSVKGRFTISRDNSK | |
| Acid | NTLYLQMNSLRAEDTAVYYCARHFTVYDGFDLWGRGTM | |
| Sequence | VTVSS | |
| H1 VLC | GACATCCAGATGACCCAGTCTCCAGGCACCCTGTCTTT | SEQ ID NO:8 |
| Nucleic | GTCTCCAGGGGAAAGAGCCACCCTCTCCTGCAGGGCCA | |
| Acid | GTCAGAGTGTTAGCAGCAGCTACTTAGCCTGGTACCAG | |
| Sequence | CAGAAACCTGGCCAGGCTCCCAGGCTCCTCATCTATGG | |
| | TGCATCCAGCAGGGCCACTGGCATCCCAGACAGGTTCA | |
| | GTGGCAGTGGGTCTGGGACAGACTTCACTCTCACCATC | |
| | AGCAGACTGGAGCCTGAAGATTTTGCAGTGTATTACTG | |
| | TCAGCAGTATGGTAGCTCACCTCGAACGTTCGGCCAAG | |
| | GGACCAAGGTGGAAATCAAA | |
| H1 VLC | DIQMTQSPGTLSLSPGERATLSCRASQSVSSSYLAWYQ | SEQ ID NO:9 |
| Amino | QKPGQAPRLLIYGASSRATGIPDRFSGSGSGTDFTLTI | |
| Acid | SRLEPEDFAVYYCQQYGSSPRTFGQGTDVEIK | |
| Sequence | | |
| H1 VHC | GAAGTTCAATTGTTAGAGTCTGGTGGCGGTCTTGTTCA | SEQ ID NO:10 |
| Nucleic | GCCTGGTGGTTCTTTACGTCTTTCTTGCGCTGCTTCCG | |
| Acid | GATTCACTTTCTCTCATTACGGTATGTCTTGGGTTCGC | |
| Sequence | CAAGCTCCTGGTAAAGGTTTGGAGTGGGTTTCTTGGAT | |
| | CGGTCCTTCTGGTGGCGCTACTCTTTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAAAGGAAGGTGGA | |
| | ATAGGGGTGGCGCCTTTGACAACTGGGGCCAGGGAACC | |
| | CTGGTCACCGTCTCAAGC | |
| H1 VHC | EVQLLESGGGLVQPGGSLRLSCAASGFTFSHYGMSWVR | SEQ ID NO:11 |
| Amino | QAPGKGLEWVSWIGPSGGATLYADSVKGRFTISRDNSK | |
| Acid | NTLYLQMNSLRAEDTAVYYCAKGRWNRGGAFDNWGQGT | |
| Sequence | LVTVSS | |
| H10 VLC | GACATCCAGATGACCCAGTCTCCACTCTCCCTGCCCGT | SEQ ID NO:12 |
| Nucleic | CACCCCTGGAGGGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| (aka | CTGACAGGTTCAGTGGCAGTGGATCAGGCACAGATTTT | |
| HAE-H10) | ACACTGAAAATCAGCAGAGTGGAGGCTGAGGATGTTGG | |
| | GGTTTATTACTGCATGCAAGCTCTGCAACCGTACACTT | |
| | TTGGCCAGGGGACCAAGCTGGAGATCAAA | |
| H10 VLC | DIQMYQSPLSLPVTPGGPASISCRSSQSLLHSNGYNYL | SEQ ID NO:13 |
| Amino | DWYLQKPGQSPQLLIYLGSNRASGVPDRFSGSGSGTDF | |
| Acid | TLKISRVEAEDVGVYYCMQALQPYTFGQGTKLEIK | |
| Sequence | | |
| H10 VHC | GAAGTTCAATTGTTAGAGTCTGGTGGCGGTCTTGTTCA | SEQ ID NO:14 |
| Nucleic | GCCTGGTGGTTCTTTACGTCTTTCTTGCGCTGCTTCCG | |
| Acid | GATTCACTTTCTCTCCTTACCTTATGTCTTGGGTTCGC | |
| Sequence | CAAGCTCCTGGTAAAGGTTTGGAGTGGGTTTCTTCTAT | |
| | CTATTCTTCTGGTGGCCTTACTGATTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACCATTGTGCGAGAGACGGTTACT | |
| | ATGATAGTAGTGGTTACGAGGGTTTTGACTACTGGGGC | |
| | CAGGGAACCCTGGTCACCGTCTCAAGC | |
| H10 VHC | EVQLLESGGGLVQPGGSLRLSCAASGFTFSPYLMSWVR | SEQ ID NO:15 |
| Amino | QAPGKGLEWVSSIYSSGGLTDYADSVKGRFTISRDNSK | |
| Acid | NTLYLQMNSLREADTAVHYCARDGYYDSSGYEGDFYWG | |
| Sequence | QGTLVTVSS | |
| A3 VLC | GACATCCAGATGACCCAGTCTCCACTCTCCCTGCCCGT | SEQ ID NO:58 |
| Nucleic | CACCCCTGGAGAGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| | CTGACAGGTTCAGTGGCAGTGGATCAGGCACAGATTTT | |
| | ACACTGAAAATCAGCAGAGTGGAGGCTGAGGATGTTGG | |
| | GGTTTATTACTGCATGCAAGCTCTACAAACTCCTCCCA | |
| | CTTTCGGCGGAGGGACCAAGGTGGAGATCAAA | |
| A3 VLC | DIQMTQSPLSLPVTPGEFASISCRSSQSLLHSNGYNYL | SEQ ID NO:59 |
| Amino | DWYLQKPGQSPQLLIYLGSNRASGVPDRFSGSGSGTDF | |
| Acid | TLKISRVEAEDVGVYYCMQALQTPPTFGGGTKVEIK | |
| Sequence | | |
| A3 VHC | GAAGTTCAATTGTTAGAGTCTGGTGGCGGTCTTGTTCA | SEQ ID NO:60 |
| Nucleic | GCCTGGTGGTTCTTTACGTCTTTCTTGCGCTGCTTCCG | |
| Acid | GATTCACTTTCTCTGAGTACGGTATGGGTTGGGTTCGC | |
| Sequence | CAAGCTCCTGGTAAAGGTTTGGAGTGGGTTTCTTCTAT | |
| | CGTTTCTTCTGGTGGCTTTACTTTTTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGAGGCACTCGTA | |
| | CAGTAACCAACTGGGGCCAGGGAGCCCTGGTCACCGTC | |
| | TCAAGC | |
| A3 VHC | EVQLLESGGGLVQPGGSLRLSCAASGFTFSEYGMGWVR | SEQ ID NO:61 |
| Amino | QAPGKGLEWVSSIVSSGGFTFYADSVKGRFTISRDNSK | |
| Acid | NTLYLQMNSLRAEDTAVYYCARGTRTVTNWGQGALVTV | |
| Sequence | SS | |
| G2 VLC | GACATCCAGATGACCCAGTCTCCACTCTCCCTGCCCGT | SEQ ID NO:62 |
| Nucleic | CACCCCTGGAGAGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| | CTGACAGGTTCAGTGGCAGTGGATCAGGCACAGATTTT | |
| | ACACTGAAAATCAGCAGAGTGGAGGCTGAGGATGTTGG | |
| | GGTTTATTACTGCATGCAAGCTCTACAAACCCCTTGGA | |
| | CTTTTGGCCAGGGGACCAAGCTGGAGATCAAA | |
| G2 VLC | DIQMTQSPLSLPVTPGEPASISVRSSQSLLHSNGYNYL | SEQ ID NO:63 |
| Amino | DWYLQKPGQSPQLLIYLGSNRASGVPDRFSGSGSGTDF | |
| Acid | TLKISRVEAEDVGVYYCMQALQTPWTFGQGTKLEIK | |
| Sequence | | |
| G2 VHC | GAAGTTCAATTGTTAGAGTCTGGTGGCGGTCTTGTTCA | SEQ ID NO:64 |
| Nucleic | GCCTGGTGGTTCTTTACGTCTTTCTTGCGCTGCTTCCG | |
| Acid | GATTCACTTTCTCTCTTTACCGTATGCGTTGGGTTCGC | |
| Sequence | CAAGCTCCTGGTAAAGGTTTGGAGTGGGTTTCTTCTAT | |
| | CTCTCCTTCTGGTGGCATTACTGAGTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGCTAGACGTGGGGG | |
| | TGGGAGCTGCTGACTACTGGGGCCAGGGAACCCTGGTC | |
| | ACCGTCTCAAGC | |
| G2 VHC | EVQLLESGGGLVQPGGSLRLSCAASGFTFSLYRMRWVR | SEQ ID NO:65 |
| Amino | QAPGKGLEWVSSISPSGGITEYADSVKGRFTISRDNSK | |
| Acid | NTLYLQMNSLRAEDTAVYYCALDVGVGAADYWGQGTLV | |
| Sequence | TVSS | |
| B12 VLC | GACATCCAGATGACCCAGTCTCCACTCTCCCTGCCCGT | SEQ ID NO:66 |
| Nucleic | CACCCCTGGAGAGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| | CTGACAGGTTCAGTGGCAGTGGATCAGGCACAGATTTT | |
| | ACACTGAAAATCAGCGGAGTGGAGGCTGAGGATGTTGG | |
| | GGTTTATTACTGCATGCAAGCTCTACAAACTGGGTACA | |
| | CTTTTGGCCAGGGGACCAAGCTGGAGATCAAA | |
| B12 VLC | DIQMTQSPLSLPVTPGEPASISCRSSQSLLHSNGYNYL | SEQ ID NO:67 |
| Amino | DWYLQKPGQSPQLLIYLGSNRASGVPDRFSGSGSGTDF | |
| Acid | TLKISGVEAEDVGVYYCMQALQTGYTFGQGTKLEIK | |
| Sequence | | |
| B12 VHC | GAAGTTCAATTGTTAGAGTCTGGTGGCGGTCTTGTTCA | SEQ ID NO:68 |
| Nucleic | GCCTGGTGGTTCTTTACGTCTTTCTTGCGCTGCTTCCG | |
| Acid | GATTCACTTTCTCTAAGTACACTATGTGGTGGGTTCGC | |
| Sequence | CAAGCTCCTGGTAAAGGTTTGGAGTGGGTTTCTTCTAT | |
| | CTGGTCTTCTGGTGGCTTTACTCGTTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGGGACGTAGTGGGA | |
| | GCTACCCCGCTGATATCTGGGGCCAAGGGACAATGGTC | |
| | ACCGTCTCAAGC | |
| B12 VHC | EVQLLESGGGLVQPGGSLRLSCAASGFTFSKYTMWWVR | SEQ ID NO:69 |
| Amino | QAPGKGLEWVSSIWSSGGFTRYADSVKGRFTISRDNSK | |
| Acid | NTLYLQMNSLRAEDTAVYYCAGRSGSYPADIWGQGTMV | |
| Sequence | TVSS | |
| D7 VLC | GACATCCAGATGACCCAGTCTCCACTCTCCCTGCCCGT | SEQ ID NO:70 |
| Nucleic | CACCCCTGGAGAGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| | CCGACAGGTTCAGTGGCAGTGGATCAGGCACAGATTTT | |
| | ACACTGAAAATCAGCAGAGTGGAGGCTGAGGATGTTGG | |
| | GGTTTATTACTGCATGCAAGCTCTACAAACTCCTAGGA | |
| | CTTTCGGCGGAGGGACCAAGGTGGAGATCAAA | |
| D7 VLC | DIQMTQSPLSLPVTPGEPASISCRSSQSLLHSNGYNYL | SEQ ID NO:71 |
| Amino | DWYLQKPGQSPQLLIYLGSNRASGVPDRFSGSGSGTDF | |
| Acid | TLKISRVEAEDVGVYYCMQALQTPRTFGGGTKVEIK | |
| Sequence | | |
| D7 VHC | GAAGTTCAATTGTTAGAGTCTGGTGGCGGTCTTGTTCA | SEQ ID NO:72 |
| Nucleic | GCCTGGTGGTTCTTTACGTCTTTCTTGCGCTGCTTCCG | |
| Acid | GATTCACTTTCTCTCATTACTCTATGATGTGGGTTCGC | |
| Sequence | CAAGCTCCTGGTAAAGGTTTGGAGTGGGTTTCTTCTAT | |
| | CTTTCCTGGTGGCTGGACTCTTTATGCTGACTCCGTTA | |
| | AAGGTCGCTTCACTATCTCTAGAGACAACTCTAAGAAT | |
| | ACTCTCTACTTGCAGATGAACAGCTTAAGGGCTGAGGA | |
| | CACTGCAGTCTACTATTGTGCGAGAGATCGGGCAGCTG | |
| | CCTACTGGGGCCAGGGAACCCTGGTCACCGTCTCAAGC | |
| D7 VHC | EVQLLESGGGLVQPGGSLRLSCAASGFTFSHYSMMWVR | SEQ ID NO:73 |
| Amino | QAPGKGLEWVSSIFPGGWTLYADSVKGRFTISRDNSKN | |
| Acid | TLYLQMNSLRAEDTAVYYCARDRAAAYWGQGTLVTVSS | |
| Sequence | | |
| BH10 VLC | GACATCCAGATGACCCAGTCTCCACTCTCCCTGCCCGT | SEQ ID NO:74 |
| Nucleic | CACCCCTGGAGAGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| | CTGACAGGTTCAGTGGCAGTGGATCAGGCACAGATTTT | |
| | ACACTGAAAATCAGCAGAGTGGAGGCTGAGGATGTTGG | |
| | GGTTTATTACTGCATGCAAGCTCTACAAACTCCCTGGA | |
| | CGTTCGGCCAAGGGACCAAGGTGGAAATCAAA | |
| BH10 VLC | DIQMTQSPLSLPVTPGEPASISCRSSQSLLHSNGYNYL | SEQ ID NO:75 |
| Amino | DWYLQKPGQSPQLLIYLGSNRASGVPDRFSGSGSGTDF | |
| Acid | TLKISRVEAEDVGVYYCMQALQTPWTFGQGTKVEIK | |
| Sequence | | |
| BH10 VHC | GAAGTTCAATTGTTAGAGTCTGGTGGCGGTCTTGTTCA | SEQ ID NO:76 |
| Nucleic | GCCTGGTGGTTCTTTACGTCTTTCTTGCGCTGCTTCCG | |
| Acid | GATTCACTTTCTCTAATTACACTATGAATTGGGTTCGC | |
| Sequence | CAAGCTCCTGGTAAAGGTTTGGAGTGGGTTTCTTCTAT | |
| | CGTTTCTTCTGGTGGCTTTACTAAGTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGAGGCTGGTCTA | |
| | GTCAGCCCGCCATCTGGGGCCAGGGAAGCCTGGTCACC | |
| | GTCTCAAGC | |
| BH10 VHC | EVQLLESGGGLVQPGGSLRLSCAASGFTFSNYTMNWVR | SEQ ID NO:77 |
| Amino | QAPGKGLEWVSSIVSSGGFTKYADSVKGRFTISRDNSK | |
| Acid | NTLYLQMNSLREADTAVYYCARGWSSQPAIWGQGSLVT | |
| Sequence | VSS | |
| B8 VLC | GACATCCAGATGACCCAGTCTCCATCCTCCCTGTCTGC | SEQ ID NO:78 |
| Nucleic | ATCTGTAGGAGACAGAGTCACCATCACTTGCCGGGCAA | |
| Acid | GTCAGAGCATTGGCAGCTATTTAAATTGGTATCAGCAG | |
| Sequence | AAACCAGGGAAAGCCCCTAAGCTCCTGATCTATGCTGC | |
| | ATCCAGTTTGCAAAGTGGGGTCCCATCAAGGTTCAGTG | |
| | GCAGTGGATCTGGGACAGATTTCACTCTCACCATCAGC | |
| | AGTCTGCAACCTGAAGATTTTGCAACTTACTACTGTCA | |
| | ACAGAGTTACTCTACCCCTCGGACTTTCGGCCCTGGGA | |
| | CCAAAGTGGATATCAAA | |
| B8 VLC | DIQMTQSPSSLSASVGDRVTITCRASQSIGSYLNWYQQ | SEQ ID NO:79 |
| Amino | KPGKAPKLLIYAASSLQSGVPSRFSGSGSGTDFTLTIS | |
| Acid | SLQPEDFATYYCQQSYSTPRTFGPGTKVDIK | |
| Sequence | | |
| B8 VHC | GAAGTTCAATTGTTAGAGTCTGGTGGCGGTCTTGTTCA | SEQ ID NO:80 |
| Nucleic | GCCTGGTGGTTCTTTACGTCTTTCTTGCGCTGCTTCCG | |
| Acid | GATTCACTTTCTCTTGGTACTCTATGTCTTGGGTTCGC | |
| Sequence | CAAGCTCCTGGTAAAGGTTTGGAGTGGGTTTCTTCTAT | |
| | CGGTCCTTCTGGTGGCCAGACTCGTTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGAGATTACTATG | |
| | ATAGTAGTGGTTATTCGTACTTTGACTACTGGGGCCAG | |
| | GGAACCCAGGTCACCGTCTCAAGC | |
| B8 VHC | EVQLLESGGGLVQPGGSLRLSCAASGFTFSWYSMSWVR | SEQ ID NO:81 |
| Amino | QAPGKGLEWVSSIGPSGGQRTYADSVKGRFTISRDNSK | |
| Acid | NTLYLQMNSLRAEDTAVYYCARDYYDSSGYSYFDYWGQ | |
| Sequence | GTQVTVSS | |
| F1 VLC | GACATCCAGATGACCCAGTCTCCACTCTCCCTGTCTGC | SEQ ID NO:82 |
| Nucleic | ATCTGTGGGAGACAGAGTCACCATCACTTGTCGGGCAA | |
| Acid | GTCAGAGCATTAGCAGCCATTTAAATTGGTATCAGCGG | |
| Sequence | AGACCAGGGAAAGCCCCTAAGCTCCTGATTTATGCTGC | |
| | ATCCAGTTTGCAAAGCGGGGTCCCATCAAGGTTCAGTG | |
| | GCAGTGGATCTGGGACAGATTTCGCTCTCACCATCAGC | |
| | AGTCTACAACCTGAAGATTTTGCAGCTTACTTCTGTCA | |
| | CCAGAGTTCCAGTACGCCTCCGACTTTCGGCCAAGGGA | |
| | CCACGGTGGAAATCAAA | |
| F1 VLC | DIQMTQSPLSLSASVGDRVTITCRASQSISSHLNWYQR | SEQ ID NO:83 |
| Amino | RPGKAPKLLIYAASSLQSGVPSRFSGSGSGTDFALTIS | |
| Acid | SLQPEDFAAYFCHQSSSTPPTFGQGTTVEIK | |
| Sequence | | |
| F1 VHC | GAAGTTCAATTGTTAGAGTCTGGTGGCGGTCTTGTTCA | SEQ ID NO:84 |
| Nucleic | GCCTGGTGGTTCTTTACGTCTTTCTTGCGCTGCTTCCG | |
| Acid | GATTCACTTTCTCTCCTTACGGTATGGATTGGGTTCGC | |
| Sequence | CAAGCTCCTGGTAAAGGTTTGGAGTGGGTTTCTTCTAT | |
| | CTCTCCTTCTGGTGGCACTACTCTTTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGACAAAAAAGGT | |
| | CCTCGTTAGGTGCTTTTGATATCTGGGGCCAAGGGACA | |
| | ATGGTCACCGTCTCAAGC | |
| F1 VHC | EVQLLESGGGLVQPGGSLRLSCAASGFTFSPYGMDWVR | SEQ ID NO:85 |
| Amino | QAPGKGLEWVSSISPSGGTTLYADSVKGRFTISRDNSK | |
| Acid | NTLYLQMNSLRAEDTAVYYCARQKRSSLGAFDIWGQGT | |
| Sequence | MVTVSS | |
| A11 VLC | GACTCAGCCTGCCTCCGTGTCTGGGTCTCCTGGACAGT | SEQ ID NO:86 |
| Nucleic | CGATCACCATCTCCTGCACTGGAACCAGCAGTGACGTT | |
| Acid | GGTGGTTATAGCTATGTCTCCTGGTACCAACAGCACCC | |
| Sequence | AGGCAAAGCCCCCAAACTCATGATTTATGAGGTCAGTA | |
| | ATCGGCCCTCTGGGGTTTCTAATCGCTTCTCTGGCTCC | |
| | AAGTCTGGCAACACGGCCTCCCTGACCATCTCTGGGCT | |
| | CCAGGCTGAAGACGAGGCTGATTATTACTGCAACTCAT | |
| | ATACAAGCAGCAGCACTAAGATGTTCGGCGGAGGGACC | |
| | AGGCTGACCGTCCTA | |
| A11 VLC | QSVLTQPASVSGSPGQSITISCTGTSSDVGGYSYVSWY | SEQ ID NO:87 |
| Amino | QQHPGKAPKLMIYEVSNRPSGVSNRFSGSKSGNTASLT | |
| Acid | ISGLQAEDEADYYCNSYTSSSTKMFGGGTRLTVL | |
| Sequence | | |
| A11 VHC | GAAGTTCAATTGTTAGAGTCTGGTGGCGGTCTTGTTCA | SEQ ID NO:88 |
| Nucleic | GCCTGGTGGTTCTTTACGTCTTTCTTGCGCTGCTTCCG | |
| Acid | GATTCACTTTCTCTAAGTACTCTATGGAGTGGGTTCGC | |
| Sequence | CAAGCTCCTGGTAAAGGTTTGGAGTGGGTTTCTCGTAT | |
| | CTATCCTTCTGGTGGCCCTACTCTTTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGAGACTCTTACG | |
| | GCATGGACGTCTGGGGCCAAGGGACCACGGTCACCGTC | |
| | TCAAGC | |
| A11 VHC | EVQLLESGGGLVQPGGSLRLSCAASGFTFSKYSMEWVR | SEQ ID NO:89 |
| Amino | QAPGKGLEWVSRIYPSGGPTLYADSVKGRFTISRDNSK | |
| Acid | NTLYLQMNSLRAEDTAVYYCARDSYGMDVWGQGTTVTV | |
| Sequence | SS | |
| H9 VLC | GACATCCAGATGACCCAGTCTCCATCCTCCCTGCCCGT | SEQ ID NO:90 |
| Nucleic | CACCCCTGGAGAGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| | CTGACAGGTTCAGTGGCAGTGGATCAGGCACAGATTTT | |
| | ACACTGAAAATCAACAGAGTGGAGGCTGAGGATGTTGG | |
| | GGTTTATTACTGCATGCAAGCTCTACAAACTCCGACGT | |
| | TCGGCCAAGGGACCAAGGTGGAAATCAAA | |
| H9 VLC | DIQMTQSPSSLPVTPGEPASISCRSSQSLLHSNGYNYL | SEQ ID NO:91 |
| Amino | DWYLQKPGQSPQLLIYLGSNRASGVPDRFSGSGSGTDF | |
| Acid | TLKINRVEAEDVGVYYCMQALQTPTFGQGTKVEIK | |
| Sequence | | |
| H9 VHC | GAAGTTCAATTGTTAGAGTCTGGTGGCGGTCTTGTTCA | SEQ ID NO:92 |
| Nucleic | GCCTGGTGGTTCTTTACGTCTTTCTTGCGCTGCTTCCG | |
| Acid | GATTCACTTTCTCTTATTACGGTATGGGTTGGGTTCGC | |
| Sequence | CAAGCTCCTGGTAAAGGTTTGGAGTGGGTTTCTTCTAT | |
| | CGGTCCTTCTGGTGGCCTTACTAATTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGAGGCACTCGTA | |
| | CAGTAACCAACTGGGGCCAGGGAACCCTGGTCACCGTC | |
| | TCAAGC | |
| H9 VHC | EVQLLESGGGLVQPGGSLRLSCAASGFTFSYYGMGWVR | SEQ ID NO:93 |
| Amino | QAPGKGLEWVSSIGPSGGLTNYADSVKGRFTISRDNSK | |
| Acid | NTLYLQMNSLRAEDTAVYYCARGTRTVTNWGQGTLVTV | |
| Sequence | SS | |
| HH10 VLC | GACATCCAGATGACCCAGTCTCCACTCTCCCTGCCCGT | SEQ ID NO:94 |
| Nucleic | CACCCCTGGAGGGCCGGCCTCCATCTCCTGCAGGTCTA | |
| Acid | GTCAGAGCCTCCTGCATAGTAATGGATACAACTATTTG | |
| Sequence | GATTGGTACCTGCAGAAGCCAGGGCAGTCTCCACAGCT | |
| | CCTGATCTATTTGGGTTCTAATCGGGCCTCCGGGGTCC | |
| | CTGACAGGTTCAGTGGCAGTGGATCAGGCACAGATTTT | |
| | ACACTGAAAATCAGCAGAGTGGAGGCTGAGGATGTTGG | |
| | GGTTTATTACTGCATGCAAGCTCTGCAACCGTACACTT | |
| | TTGGCCAGGGGACCAAGCTGGAGATCAAA | |
| HH10 VLC | DIQMYQSPLSLPVTPGGPASISFRSSQSLLHSNGTNYL | SEQ ID NO:95 |
| Amino | DWYLQKPGQSPQLLIYLGSNRASGVPDRFSGSGSGTDF | |
| Acid | TLKISRVEAEDVGVYYCMQALQPYTFGQGTKLEIK | |
| Sequence | | |
| HH10 | GAAGTTCAATTGTTAGAGTCTGGTGGCGGTCTTGTTCA | SEQ ID NO:96 |
| VHC | GCCTGGTGGTTCTTTACGTCTTTCTTGCGCTGCTTCCG | |
| Nucleic | GATTCACTTTCTCTCCTTACCTTATGTCTTGGGTTCGC | |
| Acid | CAAGCTCCTGGTAAAGGTTTGGAGTGGGTTTCTTCTAT | |
| Sequence | CTATTCTTCTGGTGGCCTTACTGATTATGCTGACTCCG | |
| | TTAAAGGTCGCTTCACTATCTCTAGAGACAACTCTAAG | |
| | AATACTCTCTACTTGCAGATGAACAGCTTAAGGGCTGA | |
| | GGACACTGCAGTCTACTATTGTGCGAGAGACGGTTACT | |
| | ATGATAGTAGTGGTTACGAGGGTTTTGACTACTGGGGC | |
| | CAGGGAACCCTGGTCACCGTCTCAAGC | |
| HH10 | EVQLLESGGGLVQPGGSLRLSCAASGFTFSPYLMSWVR | SEQ ID NO:97 |
| VHC | QAPGKGLEWVSSIYSSGGLTDYADSVKGRFTISRDNSK | |
| Amino | NYLYLQMNSLRAEDTAVYYCARDGYYDSSGYEGFDYWG | |
| Acid | QGTLVTVSS | |
| Sequence | | |

TABLE 2
LIGHT CHAIN

| | | |
|---|---|---|
| | SIGNAL SEQUENCE FR1-L CDR1-L | |
| HAE-A3-κ-light | MGWSCIILFLVATATGVHS DIQMTQSPLSLPVTPGEPASISC RSSQSLLHSNGYNYLD | |
| HAE-G2-κ-light | MGNSCIILFLVATATGVHS DIQMTQSPLSLPVTPGEPASISC RSSQSLLHSNGYNYLD | |
| HAE-H10-κ-light | MGWSCIILFLVATATGVHS DIQMTQSPLSLPVTPGGPASISC RSSQSLLHSNGYNYLD | |
| BE-B12-κ-light | MGWSCIILFLVATATGVHS DIQMTQSPLSLPVTPGEPASISC RSSQSLLHSNGYHYLD | |
| BE-D7-κ-light | MGWSCIILFLVATATGVHS DIQMTQSPLSLPVTPGEPASISC RSSQSLLHSNGYNYLD | |
| BE-H10-κ-light | MGWSCIILFLVATATGVHS DIQMTQSPLSLPVTPGEPASISC RSSQSLLHSNGYNYLD | |
| HAE-B8-κ-light | MGWSCIILFLVATATGVHS DIQHTQSPSSLSASVGDRVTITC RASQS.....IGSYLN | |
| HAE-F1-κ-light | MGWSCIILFLVATATGVHS DIQMTQSPLSLSASVGDRVTITC RASQS.....ISSHLN | |
| BE-H9-κ-light | MGWSCIILFLVATATGVHS DIQMTQSPSSLPVTPGEPASISC RSSQSLLHSNGYNYLD | |
| HAE-H-H10-κ-light | MGWSCIILFLVATATGVHS DIQHTQSPLSLPVTPGGPASISC RSSQSLLHSNGYNYLD | |
| BE-A11-λ-light | MGWSCIILFLVATATGVHS .QSVLTQPASVSGSPGQSITISC TGTSS..DVGGYSYVS | |
| | FR2-L CDR2-L FR3-L | |
| HAE-A3-light | WYLQKPGQSPQLLIY LGSNRAS GVPDRFSGSGSGTDFTLKISRVEAEDVGVYYC | |
| HAE-G2-light | WYLQKPGQSPQLLIY LGSNRAS GVPDRFSGSGSGTDFTLKISRVEAEDVGVYYC | |
| HAE-H10-light | WYLQKPGQSPQLLIY LGSNRAS GVPDRFSGSGSGTDFTLKISRVEAEDVGVYYC | |
| BE-B12-light | WYLQKPGQSPQLLIY LGSNRAS GVPDRFSGSGSGTDFTLKISGVEAEDVGVYYC | |
| BE-D7-light | WYLQKPGQSPQLLIY LGSNRAS GVPDRFSGSGSGTDFTLKISRVEAEDVGVYYC | |
| BE-H10-light | WYLQKPGQSPQLLIY LGSNRAS GVPDRFSGSGSGTDFTLKISRVEAEDVGVYYC | |
| HAE-B8-light | WYQQKPGKAPKLLIY AASSLQS GVPSRFSGSGSGTDFTLTISSLQPEDFATYYC | |
| HAE-F1-light | WYQRRPGKAPKLLIY AASSLQS GVPSRFSGSGSGTDFALTISSLQPEDFAAYFC | |
| BE-H9-light | WYLQKPGQSPQLLIY LGSNRAS GVPDRFSGSGSGTDFTLKINRVEAEDVGVYYC | |
| HAE-H-H10-light | WYLQKPGQSPQLLIY LGSNRAS GVPDRFSGSGSGTDFTLKISRVEAEDVGVYYC | |
| BE-A11-light | WYQQHPGKAPKLMIY EVSNRPS GVSNRFSGSKSGNTASLTISGLQAEDEADYYC | |
| | CDR3-L FR4-L CONSTANT REGION | |
| HAE-A3-light | MQALQT.PPT FGGGTKVEIK RTVAAPSVFIFPPSDEQLKSGTA | |
| HAE-G2-light | MQALQT.PWT FGQGTKLEIK RTVAAPSVFIFPPSDEQLKSGTA | |
| HAE-H10-light | MQALQ..PYT FGQGTKLEIK RTVAAPSVFIFPPSDEQLKSGTA | |
| BE-B12-light | MQALQT.GYT FGQGTKLEIK RTVAAPSVFIFPPSDEQLKSGTA | |
| BE-D7-light | MQALQT.PRT FGGGTKVEIK RTVAAPSVFIFPPSDEQLKSGTA | |
| BE-H10-light | MQALQT.PWT FGQGTKVEIK RTVAAPSVFIFPPSDEQLKSGTA | |
| HAE-B8-light | QQSYST.PRT FGPGTKVDIK RTVAAPSVFIFPPSDEQLKSGTA | |
| HAS-F1-light | HQSSST.PPT FGQGTTVEIK RTVAAPSVFIFFPSDEQLKSGTA | |
| BE-H9-light | MQALQT..PT FGQGTKVEIK RTVAAPSVFIFPPSDEQLKSGTA | |
| HAE-H-H10-light | MQALQ..PYT FGQGTKLEIK RTVAAPSVFIFPPSDEQLKSGTA | |
| BE-A11-light | NSYTSSSTKM FGGGTRLTVL GQPKAAPSVTLFPPSSEELQANK | |
| | CONSTANT REGION (contd) | |
| HAS-A3-light | SVVCLLNNFYPREAKVQWKVDNALQSGNSQESVTEQDSKDSTY | |
| HAE-G2-light | SVVCLLNNFYPREAKVQWKVDNALQSGNSQESVTEQDSRDSTY | |
| HAS-H10-light | SVVCLLNNFYPREAKVQWKVDNALQSGNSQESVTEQDSKDSTY | |
| BE-B12-light | SVVCLLNNFYPREAKVQWKVDNALQSGNSQESVTEQDSKDSTY | |
| BE-D7-light | SVVCLLNNFYPREAKVQWKVDNALQSGNSQESVTEQDSKDSTY | |
| BE-H10-light | SVVCLLNNFYPREAKVQWKVDNALQSGNSQESVTEQDSKDSTY | |
| HAE-B8-light | SVVCLLNNFYPREAKVQWKVDNALQSGNSQESVTEQDSKDSTY | |
| HAS-F1-light | SVVCLLNNFYPREAKVQWKVDNALQSGNSQESVTEQDSKDSTY | |
| BE-H9-light | SVVCLLNNFYPREAKVQWKVDNALQSGNSQESVTEQDSKDSTY | |
| HAE-H-H10-light | SVVCLLNNFYPREAKVQWKVDNALQSGNSQESVTEQDSKDSTY | |
| BE-A11-light | ATLVCLISDFYPGAVTVAWKADGSPVKAGVETTKPSKQSNNKY | |
| | CONSTANT REGION (contd) | |
| HAE-A3-light | SLSSTLTLSKADYEKHKVYACEVTHQGLSSPVTKSFNRGEC* | (SEQ ID NO:144) |
| HAE-G2-light | SLSSTLTLSKADYEKHKVYACEVTHQGLSSPVTKSFNRGEC* | (SEQ ID NO:145) |
| HAS-H10-light | SLSSTLTLSKADYEKHKVYACEVTHQGLSSPVTKSFNRGEC* | (SEQ ID NO:146) |
| BE-B12-light | SLSSTLTLSKADYEKHKVYACEVTHQGLSSPVTKSFNRGEC* | (SEQ ID NO:147) |
| BE-D7-light | SLSSTLTLSKADYEKHKVYACEVTHQGLSSPVTKSFNRGEC* | (SEQ ID NO:148) |
| BE-H10-light | SLSSTLTLSKADYEKHKVYACEVTHQGLSSPVTKSFNRGEC* | (SEQ ID NO:149) |
| HAE-B8-light | SLSSTLTLSKADYEKHKVYACEVTHQGLSSPVTKSFNRGEC* | (SEQ ID NO:150) |
| HAE-F1-light | SLSSTLTLSKADYEKHKVYACEVTHQGLSSPVTKSFNRGEC* | (SEQ ID NO:151) |
| BE-H9-light | SLSSTLTLSKADYEKHKVYACEVTHQGLSSPVTKSFNRGEC* | (SEQ ID NO:152) |
| HAE-H-R10-light | SLSSTLFLSKADYEKHKVYACEVTHQGLSSPVTKSFNRGEC* | (SEQ ID NO:153) |
| BE-A11-light | AASSYLSLTPEQWKSHRSYSCQVTHEG.STVEKTVAPAECS* | (SEQ ID NO:154) |

HEAVY CHAIN

| | | |
|---|---|---|
| | SIGNAL SEQUENCE FR1-H | |
| HAE-A3-heavy | MGWSCIILFLVATATGAHS EVQLLESGGGLVQPGGSLRLSCAASGFTFS | |
| HAE-G2-heavy | MGWSCIILFLVATATGAHS EVQLLESGGGLVQPGGSLRLSCAASGFTFS | |
| HAE-R10-heavy | MGWSCIILFLVATATGAHS EVQLLESGGGLVQPGGSLRLSCAASGFTFS | |
| BE-B12-heavy | MGWSCIILFLVATATGAHS EVQLLESGGGLVQPGGSLRLSCAASGFTFS | |
| BE-D7-heavy | MGWSCIILFLVATATGAHS EVQLLESGGGLVQPGGSLRLSCAASGFTFS | |
| BE-R10-heavy | MGWSCIILFLVATATGAHS EVQLLESGGGLVQPGGSLRLSCAASGFTFS | |
| HAE-B8-heavy | MGWSCIILFLVATATGAHS EVQLLESGGGLVQPGGSLRLSCAASGFTFS | |
| RAE-F1-heavy | MGWSCIILFLVATATGAHS EVQLLESGGGLVQPGGSLRLSCAASGFTFS | |
| BE-H9-heavy | MGWSCIILFLVATATGAHS EVQLLESGGGLVQPGGSLRLSCAASGFTFS | |
| RAE-H-R10-heavy | MGWSCIILFLVATATGAHS EVQLLESGGGLVQPGGSLRLSCAASGFTFS | |
| BE-A11-heavy | MGWSCIILFLVATATGAHS EVQLLESGGGLVQPGGSLRLSCAASGFTFS | |
| | CDR1-H FR2-H CDR2-H FR3-H | |
| HAE-A3-heavy | EYGMG WVRQAPGKGLEWVS SIVSSGGFTFYADSVKG RFTISRDNSKNT | |
| HAE-G2-heavy | LYRMR WVRQAPGKGLEWVS SISPSGGITEYADSVKG RETISRDNSKNT | |
| RAE-R10-heavy | PYLMS WVRQAPGKGLEWVS SIYSSGGLTDYADSVRG RETISRDNSKNT | |
| BE-B12-heavy | KYTMW WVRQAPGKGLEWVS SIWSSGGFTRYADSVKG RFTISRDNSKNT | |
| BE-D7-heavy | HYSMN WVRQAPGKGLEWVS SIFP.GGWTLYADSVKG RFTISRDNSKNT | |
| BE-R10-heavy | NYTMN WVRQAPGKGLEWVS SIVSSGGFTKYADSVRG RFTISRDNSKNT | |
| HAE-B8-heavy | WYSMS WVRQAPGKGLEWVS SIGPSGGQTRYADSVKG RFTISRDNSKNT | |
| HAE-F1-heavy | PYGMD WVRQAPGKGLEWVS SISPSGGTTLYADSVKG RFTISRDNSKNT | |
| BE-R9-heavy | YYGMG WVRQAPGKGLEWVS SIGPSGGLTNYADSVKG RFTISRDNSKNT | |
| HAE-H-R10-heavy | PYLMS WVRQAPGKGLEWVS SIYSSGGLTDYADSVKG RFTISRDNSKNT | |
| BE-A11-heavy | KYSME WVRQAPGKGLEWVS RIYPSGGPTLYADSVKG RFTISRDNSKNT | |
| | FR3-H(contd) CDR3-H FR4-H | |
| HAE-A3-heavy | LYLQMNSLRAEDTAVYYCAR G.......TRTVT NWGQGALVTVSS | |
| HAE-G2-heavy | LYLQMNSLRAEDTAVYYCAL D.....VGVGAAD YWGQGTLVTVSS | |
| HAE-R10-heavy | LYLQMNSLRAEDTAVYHCAR DGYYDSSGYEGFD YWGQGTLVTVSS | |
| BE-B12-heavy | LYLQMNSLRAEDTAVYYCAG R.....SGSYPAD IWGQGTMVTVSS | |
| BE-D7-heavy | LYLQMNSLRAEDTAVYYCAR D........RAAA YWGQGTLVTVSS | |
| BE-H10-heavy | LYLQMNSLRAEDTAVYYCAR G......WSSQPA IWGQGSLVTVSS | |
| HAE-B8-heavy | LYLQMNSLRAEDTAVYYCAR D.YYDSSGYSYFD YWGQGTQVTVSS | |
| HAE-F1-heavy | LYLQMNSLRAEDTAVYYCAR Q...KRSSLGAFD IWGQGTMVTVSS | |
| BE-R9-heavy | LYLQMNSLRAEDTAVYYCAR G.......TRTVT NWGQGTLVTVSS | |
| HAE-H-H10-heavy | LYLQMNSLRAEDTAVYYCAR DGYYDSSGYEGFD YWGQGTLVTVSS | |
| BE-A11-heavy | LYLQMNSLRAEDTAVYYCAR D.......SYGMD VWGQGTTVTVSS | |
| | CONSTANT REGION-H IgG4 | |
| HAE-A3-heavy | ASTKGPSVFPLAPCSRSTSESTAALGCLVKDYFPEPVTVSWNSGALTSGVHTF | |
| HAE-G2-heavy | ASTKGPSVFPLAPCSRSTSESTAALGCLVKDYFPEPVTVSWNSGALTSGVHTF | |
| HAE-H10-heavy | ASTKGPSVFPLAPCSRSTSESTAALGCLVKDYFPEPVTVSWNSGALTSGVHTF | |
| BE-B12-heavy | ASTKGPSVFPLAPCSRSTSESTAALGCLVKDYFPEPVTVSWNSGALTSGVHTF | |
| BE-D7-heavy | ASTKGPSVFPLAPCSRSTSESTAALGCLVKDYFPEPVTVSWNSGALTSGVHTF | |
| BE-R10-heavy | ASTKGPSVFPLAPCSRSTSESTAALGCLVKDYFPEPVTVSWNSGALTSGVHTF | |
| HAE-B8-heavy | ASTKGPSVFPLAPCSRSTSESTAALGCLVKDYFPEPVTVSWNSGALTSGVHTF | |
| RAE-F1-heavy | ASTKGPSVFPLAPCSRSTSESTAALGCLVKDYFPEPVTVSWNSGALTSGVHTF | |
| BE-H9-heavy | ASTKGPSVFPLAPCSRSTSESTAALGCLVKDYFPEPVTVSWNSGALTSGVHTF | |
| HAE-H-H10-heavy | ASTKGPSVFPLAPCSRSTSESTAALGCLVKDYFPEPVTVSWNSGALTSGVHTF | |
| BE-A11-heavy | ASTKGPSVFPLAPCSRSTSESTAALGCLVKDYFPEPVTVSWNSGALTSGVHTF | |
| | CONSTANT REGION-H (contd) | |
| HAE-A3-heavy | PAVLQSSGLYSLSSVVTVPSSSLGTKTYTCNVDHKPSNTKVDKRVESKYGPPC | |
| HAE-G2-heavy | PAVLQSSGLYSLSSVVTVPSSSLGTKTYTCNVDHKPSNTKVDKRVESKYGPPC | |
| HAE-H10-heavy | PAVLQSSGLYSLSSVVTVPSSSLGTKTYTCNVDHKPSNTKVDKRVESKYGPPC | |
| BE-B12-heavy | PAVLQSSGLYSLSSVVTVPSSSLGTKTYTCNVDHKPSNTKVDKRVESKYGPPC | |
| BE-D7-heavy | PAVLQSSGLYSLSSVVTVPSSSLGTKTYTCNVDHKPSNTKVDKRVESKYGPPC | |
| BE-H10-heavy | PAVLQSSGLYSLSSVVTVPSSSLGTKTYTCNVDRKPSNTKVDKRVESKYGPPC | |
| HAE-B8-heavy | PAVLQSSGLYSLSSVVTVPSSSLGTKTYTCNVDRKPSNTKVDKRVESKYGPPC | |
| HAE-F1-heavy | PAVLQSSGLYSLSSVVTVPSSSLGTKTYTCNVDRKPSNTKVDKRVESKYGPPC | |
| BE-H9-heavy | PAVLQSSGLYSLSSVVTVPSSSLGTKTYTCNVDRKPSNTKVDKRVESKYGPPC | |
| HAE-H-H10-heavy | PAVLQSSGLYSLSSVVTVPSSSLGTKTYTCNVDRKPSNTKVDKRVESKYGPPC | |
| BE-A11-heavy | PAVLQSSGLYSLSSVVTVPSSSLGTKTYTCNVDRKPSNTKVDKRVESKYGPPC | |
| | CONSTANT REGION-H (contd) | |
| HAE-A3-heavy | PSCPAPEFLGGPSVFLFPPKPKDTLMISRTPEVTCVVVDVSQEDPEVQFNWYV | |
| HAE-G2-heavy | PSCPAPEFLGGPSVFLFPPKPKDTLMISRTPEVTCVVVDVSQEDPEVQFNWYV | |
| HAE-H10-heavy | PSCPAPEFLGGPSVFLFPPKPKDTLMISRTPEVTCVVVDVSQEDPEVQFNWYV | |
| BE-B12-heavy | PSCPAPEFLGGPSVFLFPPKPKDTLMISRTPEVTCVVVDVSQEDPEVQFNWYV | |
| BE-D7-heavy | PSCPAPEFLGGPSVFLFPPKPKDTLMISRTPEVTCVVVDVSQEDPEVQFNWYV | |
| BE-H10-heavy | PSCPAPEFLGGPSVFLFPPKPKDTLMISRTPEVTCVVVDVSQEDPEVQFNWYV | |
| HAE-B8-heavy | PSCPAPEFLGGPSVFLFPPKPKDTLMISRTPEVTCVVVDVSQEDPEVQFNWYV | |
| HAE-F1-heavy | PSCPAPEFLGGPSVFLFPPKPKDTLMISRTPEVTCVVVDVSQEDPEVQFNWYV | |
| BE-H9-heavy | PSCPAPEFLGGPSVFLFPPKPKDTLMISRTPEVTCVVVDVSQEDPEVQFNWYV | |
| HAE-H-H10-heavy | PSCPAPEFLGGPSVFLFPPKPKDTLMISRTPEVTCVVVDVSQEDPEVQFNWYV | |
| BE-A11-heavy | PSCPAPEFLGGPSVFLFPPKPKDTLMISRTPEVTCVVVDVSQEDPEVQFNWYV | |
| | CONSTANT REGION-H (contd) | |
| HAE-A3-heavy | DGVEVHNAKTKPREEQFNSTYRVVSVLTVLHQDWLNGKEYKCKVSNKGLPSSI | |
| HAE-G2-heavy | DGVEVHNAKTKPREEQFNSTYRVVSVLTVLHQDWLNGKEYKCKVSNKGLPSSI | |
| HAE-H10-heavy | DGVEVHNAKTKPREEQFNSTYRVVSVLTVLHQDWLNGKEYKCKVSNKGLPSSI | |
| BE-B12-heavy | DGVEVHNAKTKPREEQFNSTYRVVSVLTVLHQDWLNGKEYKCKVSNKGLPSSI | |
| BE-D7-heavy | DGVEVHNAKTKPREEQFNSTYRVVSVLTVLHQDWLNGKEYKCKVSNKGLPSSI | |
| BE-H10-heavy | DGVEVHNAKTKPREEQFNSTYRVVSVLTVLHQDWLNGKEYKCKVSNKGLPSSI | |
| HAE-B8-heavy | DGVEVHNAKTKPREEQFNSTYRVVSVLTVLHQDWLNGKEYKCKVSNKGLPSSI | |
| HAE-F1-heavy | DGVEVHNAKTKPREEQFNSTYRVVSVLTVLHQDWLNGKEYKCKVSNKGLPSSI | |
| BE-H9-heavy | DGVEVHNAKTKPREEQFNSTYRVVSVLTVLHQDWLNGKEYKCKVSNKGLPSSI | |
| HAE-H-H10-heavy | DGVEVHNAKTKPREEQFNSTYRVVSVLTVLHQDWLNGKEYKCKVSNKGLPSSI | |
| BE-A11-heavy | DGVEVHNAKTKPREEQFNSTYRVVSVLTVLHQDWLNGKEYKCKVSNKGLPSSI | |
| | CONSTANT REGION-H (contd) | |
| HAE-A3-heavy | EKTISKAKGQPREPQVYTLPPSQEEMTKNQVSLTCLVKGFYPSDIAVEWESNG | |
| HAE-G2-heavy | EKTISKAKGQPREPQVYTLPPSQEEMTKNQVSLTCLVKGFYPSDIAVEWESNG | |
| RAE-H10-heavy | EKTISKAKGQPREPQVYTLPPSQEEMTKNQVSLTCLVKGFYPSDIAVEWESNG | |
| BE-B12-heavy | EKTISKAKGQPREPQVYTLPPSQEEMTKNQVSLTCLVKGFYPSDIAVEWESNG | |
| BE-D7-heavy | EKTISKAKGQPREPQVYTLPPSQEEMTKNQVSLTCLVKGFYPSDIAVEWESNG | |
| BE-H10-heavy | EKTISKAKGQPREPQVYTLPPSQEEMTKNQVSLTCLVKGFYPSDIAVEWESNG | |
| HAE-B8-heavy | EKTISKAKGQPREPQVYTLPPSQEEMTKNQVSLTCLVKGFYPSDIAVEWESNG | |
| HAE-F1-heavy | EKTISKAKGQPREPQVYTLPPSQEEMTKNQVSLTCLVKGFYPSDIAVEWESNG | |
| BE-H9-heavy | EKTISKAKGQPREPQVYTLPPSQEEMTKNQVSLTCLVKGFYPSDIAVEWESNG | |
| HAE-H-H10-heavy | EKTISKAKGQPREPQVYTLPPSQEEMTKNQVSLTCLVKGFYPSDIAVEWESNG | |
| BE-A11-heavy | EKTISKAKGQPREPQVYTLPPSQEEMTKNQVSLTCLVKGFYPSDIAVEWESNG | |
| | CONSTANT REGION-H (contd) | |
| HAE-A3-heavy | QPENNYKTTPPVLDSDGSFFLYSRLTVDKSRWQEGNVFSCSVMHEALHNHYTQ | |
| HAE-G2-heavy | QPENNYKTTPPVLDSDGSFFLYSRLTVDKSRWQEGNVFSCSVMHEALHNHYTQ | |
| HAE-H10-heavy | QPENNYKTTPPVLDSDGSFFLYSRLTVDKSRWQEGNVFSCSVMHEALHNHYTQ | |
| BE-B12-heavy | QPENNYKTTPPVLDSDGSFFLYSRLTVDKSRWQEGNVFSCSVMHEALHNHYTQ | |
| BE-D7-heavy | QPENNYKTTPPVLDSDGSFFLYSRLTVDKSRWQEGNVFSCSVMHEALHNHYTQ | |
| BE-H10-heavy | QPENNYKTTPPVLDSDGSFFLYSRLTVDKSRWQEGNVFSCSVMHEALHNHYTQ | |
| HAE-B8-heavy | QPENNYKTTPPVLDSDGSFFLYSRLTVDKSRWQEGNVFSCSVMHEALHNHYTQ | |
| HAE-F1-heavy | QPENNYKTTPPVLDSDGSFFLYSRLTVDKSRWQEGNVFSCSVMHEALHNHYTQ | |
| BE-H9-heavy | QPENNYKTTPPVLDSDGSFFLYSRLTVDKSRWQEGNVFSCSVMHEALHNHYTQ | |
| HAE-H-H10-heavy | QFENNYKTTPPVLDSDGSFFLYSRLTVDKSRWQEGNVFSCSVMHEALHNHYTQ | |
| BE-A11-heavy | QPENNYKTTPPVLDSDGSFFLYSRLTVDKSRWQEGNVFSCSVMHEALHNHYTQ | |
| HAE-A3-heavy | KSLSLSLGK* | (SEQ ID NO:155) |
| HAE-G2-heavy | KSLSLSLGK* | (SEQ ID NO:156) |
| HAE-H10-heavy | KSLSLSLGK* | (SEQ ID NO:157) |
| BE-B12-heavy | KSLSLSLGK* | (SEQ ID NO:158) |
| BE-D7-heavy | KSLSLSLGK* | (SEQ ID NO:159) |
| BE-H10-heavy | KSLSLSLGK* | (SEQ ID NO:160) |
| HAE-B8-heavy | KSLSLSLGK* | (SEQ ID NO:161) |
| HAE-F1-heavy | KSLSLSLGK* | (SEQ ID NO:162) |
| BE-H9-heavy | KSLSLSLGK* | (SEQ ID NO:163) |
| HAE-H-H10-heavy | KSLSLSLGK* | (SEQ ID NO:164) |
| BE-A11-heavy | KSLSLSLGK* | (SEQ ID NO:165) |

TABLE 3

| | | |
|---|---|---|
| Antibody | Sequence | Identifier |

| | | |
|---|---|---|
| F2 VLC CDR1 | RSSESLVYSDGNTYLG | SEQ ID NO:16 |
| F2 VLC CDR2 | KVSNRDS | SEQ ID NO:17 |
| F2 VLC CDR3 | MHSIRWPWT | SEQ ID NO:18 |
| F2 VLC FR1 | DIQMTQSPLSLAVTLGQPASTSC | SEQ ID NO:19 |
| F2 VLC FR2 | WFQQRPGQSPRRLLY | SEQ ID NO:20 |
| F2 VLC FR3 | GVPDRFSGSGSGTDFTLHISRVEAEDVGVYYC | SEQ ID NO:21 |
| F2 VLC FR4 | FGQGTTVEIK | SEQ ID NO:22 |
| F2 VHC CDR1 | PYTMA | SEQ ID NO:23 |
| F2 VHC CDR2 | SIYPSGGTTPYADSVKG | SEQ ID NO:24 |
| F2 VHC CDR3 | HFTVYDGFD | SEQ ID NO:25 |
| F2 VHC FR1 | EVQLLESGGGLVQPGGSLRLSCAASGFTFS | SEQ ID NO:26 |
| F2 VHC FR2 | WVRQAPGKGLEWVS | SEQ ID NO:27 |
| F2 VHC FR3 | RFTISRDNSKNTLYLQMNSLREADTAVYYCAR | SEQ ID NO:28 |
| F2 VHC FR4 | LWGRGTMVTVSS | SEQ ID NO:29 |
| H1 VLC CDR1 | RASQSVSSSYLA | SEQ ID NO:30 |
| H1 VLC CDR2 | GASSRAT | SEQ ID NO:31 |
| H1 VLC CDR3 | QQYGSSPRT | SEQ ID NO:32 |
| H1 VLC FR1 | DIQMTQSPGTLSLSPGERATLSC | SEQ ID NO:33 |
| H1 VLC FR2 | WIGPSGGATLYADSVKG | SEQ ID NO:34 |
| H1 VLC FR3 | GIPDRFSGSGSGTDFTLTISRLEPEDFAVYYC | SEQ ID NO:35 |
| H1 VLC FR4 | FGQGTKVEIK | SEQ ID NO:36 |
| H1 VHC CDR1 | HYGMS | SEQ ID NO:37 |
| H1 VHC CDR2 | WIGPSGGATLYADSVKG | SEQ ID NO:38 |
| H1 VHC CDR3 | GRWNRGGAFD | SEQ ID NO:39 |
| H1 VHC FR1 | EVQLLESGGGLVQPGGSLRLSCAASGFTFS | SEQ ID NO:40 |
| H1 VHC FR2 | WVRQAPGKGLEWVS | SEQ ID NO:41 |
| H1 VHC FR3 | RFTISRDNDKNTLYLQMNSLRAEDTAVYYCAR | SEQ ID NO:42 |
| H1 VHC FR4 | NWGQGTLVTVSS | SEQ ID NO:43 |
| H10 VLC CDR1 | RSSQSLLHSNGYNYLD | SEQ ID NO:44 |
| H10 VLC CDR2 | LGSNRAS | SEQ ID NO:45 |
| H10 VLC CDR3 | MQALQPYT | SEQ ID NO:46 |
| H10 VLC FR1 | DIQMTQSPLSLPVTPGGPASISC | SEQ ID NO:47 |
| H10 VLC FR2 | WYLQKPGQSPQLLIY | SEQ ID NO:48 |
| H10 VLC FR3 | GVPDRFSGSGSGTDFTLKISRVEAEDVGVYYC | SEQ ID NO:49 |
| H10 VLC FR4 | FGQGTKLEIK | SEQ ID NO:50 |
| H10 VHC CDR1 | PYLMS | SEQ ID NO:51 |
| H10 VHC CDR2 | SIYSSGGLTDYADSVKG | SEQ ID NO:52 |
| H10 VHC CDR3 | DGYYDSSGYEGFD | SEQ ID NO:53 |
| H10 VHC FR1 | EVQLLESGGGLVQPGGSLRLSCAASGFTFS | SEQ ID NO:54 |
| H10 VHC FR2 | WVRQAPGKGLEWVS | SEQ ID NO:55 |
| H10 VHC FR3 | RFTISRDNSKNTLYLQMNSLRAEDTAVYHCAR | SEQ ID NO:56 |
| H10 VHC FR4 | YWGQGTLVTVSS | SEQ ID NO:57 |

* Additional CDRs and Framework regions are detailed in Table 2, above.

In a preferred embodiment, the antibody includes at least one, two and preferably three CDR's from the light or heavy chain variable region of an antibody disclosed herein, e.g., BE-B12, BE-D7, HAE-B8, HAE-F1, BE-A11, F2, HAE-H10, HAE-H-H10, H1, HAE-A3, BE-H10, HAE-H9, or HAE-G2, or a sequence substantially identical thereto. In other embodiments, the antibody can have at least one, two and preferably three CDR's from the light or heavy chain variable region of an antibody disclosed herein, e.g., BE-B12, BE-D7, HAE-B8, HAE-F1, BE-A11, F2, HAE-H10, HAE-H-H10, H1, HAE-A3, BE-H10, HAE-H9, or HAE-G2, as produced by their respective clones. In one preferred embodiment, the antibody includes all six CDR's from a human CD44-binding antibody disclosed herein, e.g., BE-B12, BE-D7, HAE-B8, HAE-F1, BE-A11, F2, HAE-H10, HAE-H-H10, H1, HAE-A3, BE-H10, HAE-H9, or HAE-G2. The CDR and framework sequences of antibodies F2, H1, and H10are shown, e.g., in Table 3; the CDR and framework sequences of antibodies G2, H10, and A3 and other antibodies are shown, e.g., in Table 2. Note that the heavy chains of HAE-H10 and HAE-H-H10differ by one amino acid in FR3. In one embodiment, the constant region of a CD44-binding antibody differs from the constant region shown in Table 2.

In another preferred embodiment, the antibody includes at least one, two and preferably three CDR's from the light or heavy chain variable region of an antibody disclosed herein, e.g., BE-B12, BE-D7, HAE-B8, HAE-F1, BE-A11, F2, HAE-H10, HAE-H-H10, H1, HAE-A3, BE-H10, HAE-H9, or HAE-G2, having an amino acid sequence that differs by no more than 3, 2.5, 2, 1.5, 1, or 0.5 substitutions, insertions or deletions for every 10 amino acids relative to the corresponding CDRs of the disclosed antibody. Further, the antibody, or antigen-binding fragment thereof, can include six CDR's, each of which differs by no more than 3, 2.5, 2, 1.5, 1, or 0.5 substitutions, insertions or deletions for every 10 amino acids relative to the corresponding CDRs of a human CD44-binding antibody disclosed herein, e.g., BE-B12, BE-D7, HAE-B8, HAE-F1, BE-A11, F2, HAE-H10, HAE-H-H10, H1, HAE-A3, BE-H10, HAE-H9, or HAE-G2.

In another embodiment, the light or heavy chain immunoglobulin of the CD44-binding antibody, can further include a light or a heavy chain variable framework that has no more than 3, 2.5, 2, 1.5, 1, or 0.5 substitutions, insertions or deletions for every 10 amino acids in FR1, FR2, FR3, or FR4 relative to the corresponding frameworks of an antibody disclosed herein, e.g., BE-B12, BE-D7, HAE-B8, HAE-F1, BE-A11, F2, HAE-H10, HAE-H-H10, H1, HAE-A3, BE-H10, HAE-H9, or HAE-G2. In a preferred embodiment, the light or heavy chain immunoglobulin of the CD44-binding antibody, or antigen-binding fragment thereof, further includes a light or a heavy chain variable framework, e.g., FR1, FR2, FR3, or FR4, that is identical to a framework of an antibody disclosed herein, e.g., BE-B12, BE-D7, HAE-B8, HAE-F1, BE-A11, F2, HAE-H10, HAE-H-H10, H1, HAE-A3, BE-H10, HAE-H9, or HAE-G2.

In one embodiment, the light or the heavy chain variable framework can be chosen from: (a) a light or heavy chain variable framework including at least 80%, 90%, 95%, or preferably 100% of the amino acid residues from a human light or heavy chain variable framework, e.g., a light or heavy chain variable framework residue from a human mature antibody or a human germline sequence (e.g., VH3, e.g., 3/23), or a consensus sequence (see, e.g., U.S. Pat. No. 6,300,064); (b) a light or heavy chain variable framework including from 20% to 80%, 40% to 80%, or 60% to 90% of the amino acid residues from a human light or heavy chain variable framework, e.g., a light or heavy chain variable framework residue from a human mature antibody or a human germline sequence, or a consensus sequence; (c) a non-human framework (e.g., a rodent framework); (d) a non-human framework that has been modified, e.g., to remove antigenic or cytotoxic determinants, e.g., deimmunized, or partially humanized. In a preferred embodiment, the antibody does not induce an immune response (e.g., a response that is detectable and/or adverse) in a human subject; or (e) an immunoglobulin framework of any species that includes an amino acid of human origin at one or more of the following positions: 4L, 38L, 43L, 44L, 58L, 62L,65L, 66L, 67L, 68L, 69L, 73L, 85L, 98L, 2H, 4H, 36H, 39H, 43H, 45H, 69H, 70H, 74H, and/or 92H (according to the Kabat numbering), or a human consensus amino acid at one or more of the following positions (preferably at least five, ten, twelve, or all): (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering).

In one embodiment, the heavy chain framework includes an amino acid sequence that is at least 70%, 80%, 85%, 90%, 95%, 97%, 98%, or 99% identical (e.g., 100%) to the heavy chain framework of, e.g., an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G; or which differs by at least 1 to 5, but by less than 40, 30, 20, 10, or 6 residues from the heavy chain framework sequence of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G. In another embodiment, the light chain framework includes an amino acid sequence that is at least 70%, 80%, 85%, 90%, 95%, 97%, 98%, or 99% identical (e.g., 100%) to the light chain framework of an antibody described herein, e.g., BE-B12, BE-D7, HAE-B8, HAE-F1, BE-A11, F2, HAE-H10, HAE-H-H10, H1, HAE-A3, BE-H 10, HAE-H9, or HAE-G2; or which differs by at least 1 to 5, but by less than 40, 30, 20, 10, or 6 residues from the light chain framework sequence of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G.

In other embodiments, the modified heavy chain variable region of the CD44 antibody has an amino acid sequence that is at least 70%, 80%, 85%, 90%, 95%, 97%, 98%, 99%, or more (e.g., 100%), identical to, e.g., a corresponding region of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G; or which differs by at least 1 to 5, but by less than 40, 30, 20, 10, or 6 residues from the amino acid sequence of a corresponding region of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G. In other embodiments, the modified light chain variable region of the CD44 antibody has an amino acid sequence, which is at least 80%, 85%, 90%, 95%, 97%, 98%, 99%, or more (e.g., 100%), identical to, e.g., a corresponding region of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G; or which differs by at least 1 to 5, but by less than 40, 30, 20, or 10 residues from the amino acid sequence of a corresponding region of an antibody described herein, e.g., BE-B12, BE-D7, HAE-B8, HAE-F1, BE-A1, F2, HAE-H10, HAE-H-H10, H1, HAE-A3, BE-H10, HAE-H9, or HAE-G.

Preferred CD44-binding antibodies include at least one, preferably two, light chain variable regions having the amino acid sequence of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G, and at least one, preferably two, heavy chain variable regions having the amino acid sequence of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G.

In other embodiments, the light or heavy chain variable framework of the CD44-binding antibody, or antigen-binding fragment thereof, includes at least one, two, three, four, five, six, seven, eight, nine, ten, fifteen, sixteen, or seventeen amino acid residues from a human light or heavy chain variable framework, e.g., a light or heavy chain variable framework residue from a human mature antibody, a human germline sequence, or a consensus sequence. In one embodiment, the amino acid residue from the human light chain variable framework is the same as the residue found at the same position in a human germline. Preferably, the amino acid residue from the human light chain variable framework is the most common residue in the human germline at the same position.

Also within the scope of the invention are antibodies, or antigen-binding fragments thereof, which bind overlapping epitopes of, or competitively inhibit, the binding of the CD44-binding peptides or antibodies disclosed herein to CD44, e.g., antibodies which bind overlapping epitopes of, or competitively inhibit, the binding of monospecific antibodies, e.g., BE-B12, BE-D7, HAE-B8, HAE-Fi, BE-A11, F2, HAE-H10, HAE-H-H10, H1, HAE-A3, BE-H10, HAE-H9, or HAE-G2, to CD44. Any combination of CD44-binding antibodies is within the scope of the invention, e.g., two or more antibodies that bind to different regions of CD44, e.g., antibodies that bind to two different epitopes on the extracellular domain of CD44, e.g., a bispecific antibody.

In one embodiment, the CD44-binding antibody, or antigen-binding fragment thereof, includes at least one light or heavy chain immunoglobulin (or preferably, at least one light chain immunoglobulin and at least one heavy chain immunoglobulin). Preferably, each immunoglobulin includes a light or a heavy chain variable region having at least one, two and, preferably, three complementarity determining regions (CDR's) substantially identical to a CDR from a CD44-binding light or heavy chain variable region, respectively, i.e., from a variable region of an antibody described herein, e.g. BE-B12, BE-D7, HAE-B8, HAE-F1, BE-A11, F2, HAE-H10, HAE-H-H10, H1, HAE-A3, BE-H10, HAE-H9, or HAE-G2.

An CD44-binding ligand described herein can be used alone, e.g., can be administered to a subject or used in vitro in non-derivatized or unconjugated forms. In other embodiments, the CD44-binding ligand can be derivatized, modified or linked to another functional molecule, e.g., another polypeptide, protein, isotope, cell, or insoluble support. For example, the CD44-binding ligand can be functionally linked (e.g., by chemical coupling, genetic fusion, non-covalent association or otherwise) to one or more other molecular entities, such as an antibody (e.g., if the ligand is an antibody to form a bispecific or a multispecific antibody), a toxin, a radioisotope, a therapeutic (e.g., a cytotoxic or cytostatic) agent or moiety, among others. For example, the CD44-binding ligand can be coupled to a radioactive ion (e.g., an α-, γ-, or β-emitter), e.g., iodine (¹³¹I or ¹²⁵I), yttrium (⁹⁰Y), lutetium (¹⁷⁷Lu), actinium (²²⁵Ac), rhenium (¹⁸⁶Re), or bismuth (²¹² or ²¹³Bi).

In another aspect, the invention features a nucleic acid that includes a coding sequence that encodes a polypeptide comprising an immunoglobulin heavy chain variable domain that binds to CD44, e.g., an immunoglobulin heavy chain variable domain described herein. For example, the immunoglobulin heavy chain variable domain can include: (a) a CDR1 sequence motif comprising X₁—Y—X₂-M-X₃ (SEQ ID NO:______), wherein X₁ is E, L or P, X₂ is G, R, or L, and X₃ is G, R, or S, (b) a CDR2 sequence comprising: S—I—X₁—X₂—S-G-G-X₃-T-X₄—Y-A-D-S—V—K-G, where X₁ is any amino acid, X₂ is any amino acid, X₃ is hydrophobic, and X₄ is any amino acid, and/or (c) a CDR3 sequence comprising one of the following exemplary sequences: DVGVGAAD (SEQ ID NO:______), DGYYDSSGYEGFD (SEQ ID NO:______), and GTRTVT (SEQ ID NO:______). In another example, the immunoglobulin heavy chain variable domain can include: a HC CDR₁ sequence motif comprising X₁—I—Y—X₂-M-X₃ (SEQ ID NO:______), wherein X₁ is any amino acid (e.g., E, L, K, H, N, W, or P), X₂ is any amino acid (e.g., G, R, S, T, or L), and X₃ is any amino acid (e.g., G, R, W, M, N, D, E, or S); a HC CDR₂ sequence comprising: X₁—I—X₂'X₃—X₄-G-G-X₅-T-X₆—Y-A-D-S—V—K-G (SEQ ID NO:______), where X₁ is any amino acid (e.g., S or R); X₂ is any amino acid (e.g., V, S, Y, W, F, V, G. or S), X₃ is any amino acid (e.g., S or P), X₄ is S or absent, X₅ is hydrophobic (e.g., F, I, L, W, or P) or Q or T, and X₆ is any amino acid (e.g., F, E, D, R, L, or K); and/or a HC CDR3 sequence comprising one of the following exemplary sequences: DVGVGAAD (SEQ ID NO:______), DGYYDSSGYEGFD (SEQ ID NO:______), RSGSYPAD (SEQ ID NO:______), DRAAA (SEQ ID NO:______), GWSSQPA (SEQ ID NO:______), DYYDSSGYSYFD (SEQ ID NO:______), QKRSSLGAFD (SEQ ID NO:_______), DSYGMD (SEQ ID NO:______) and GTRTVT (SEQ ID NO:______), or a sequence that is at least 85% identical to one of the foregoing. The immunoglobulin heavy chain variable domain can include a framework region described herein. In one example, the variable domain is a heavy chain variable domain is at least 75, 80, 85, 90, 92, 95, 96, 97, 98, or 99% identical to a heavy chain variable domain of an antibody described herein, e.g., BE-B12, BE-D7, HAE-B8, HAE-F1, BE-A11, F2, HAE-H10, HAE-H-H10, H1, HAE-A3, BE-H10, HAE-H9, or HAE-G.

In another aspect, the invention features a nucleic acid that includes a coding sequence that encodes a polypeptide comprising an immunoglobulin light chain variable domain that binds to CD44, e.g., an immunoglobulin light chain variable domain described herein. For example, the immunoglobulin light chain variable domain can include: (a) a CDR1 comprising an amino acid sequence of at least 13amino acids of which at least 11 amino acids are identical to RSSQSLLHSNGYNYLD (SEQ ID NO:______); (b) a CDR2 comprising an amino acid sequence of at least 7 amino acids of which at least 5 amino acids are identical to LGSNRAS (SEQ ID NO:______); and/or (c) a CDR3 comprising M-Q-A-L-Q-X1-P-X2-T (SEQ ID NO:______), where X₁ is any amino acid or no amino acid, and X2 is any amino acid (e.g., hydrophobic, R, or Y) or absent.

The immunoglobulin light chain variable domain can include a framework region described herein. In one example, the variable domain is a light chain variable domain is at least 75, 80, 85, 90, 92, 95, 96, 97, 98, or 99% identical a corresponding light chain variable domain of an antibody described herein, e.g., BE-B12, BE-D7, HAE-H8, HAE-F1, BE-A11, F2, HAE-H10, HAE-H-H10, H1, HAE-A3, BE-H10, HAE-H9, or HAE-G.

A nucleic acid described herein can further include a promoter operably linked to the coding sequence. A nucleic acid can include a first and second coding sequence, e.g., wherein the first coding sequence encodes a polypeptide that includes an immunoglobulin heavy chain variable domain and the second coding sequence encodes a polypeptide that includes an immunoglobulin light chain variable domain.

In another aspect, the invention features a host cell that contains a first nucleic acid encoding a polypeptide comprising a heavy chain variable region and a second nucleic acid encoding a polypeptide comprising a light chain variable region. The heavy chain variable region and the light chain variable region can associate to form a CD44 binding protein. These sequences encoding variable regions can have one or more properties described herein, e.g., at least 75, 80, 85, 90, 92, 95, 96, 97, 98, or 99% identity (e.g., 100%) to a sequence described herein, or the ability to hybridize to a nucleic acid sequence described herein or a complement thereof, or can encode a variable region described herein. The invention also includes a method of providing a CD44-binding antibody. The method can include providing a host cell described herein; and expressing said first and second nucleic acids in the host cell under conditions that allow assembly of said light and heavy chain variable regions to form an antigen binding protein that interacts with CD44.

In one embodiment, the protein ligand at the concentration corresponding to its Kd for CD44 causes at least 2, 4, 8, or 20 fold greater lysis in the CD44 agonist assay (as defined herein) than the amount of lysis observed in the absence of the protein ligand.

In another aspect, the invention features a protein that includes a human (or humanized) heavy chain immunoglobulin variable domain and a human (or humanized) light chain immunoglobulin variable domain. The protein binds to human CD44 ectodomain with a K_{d} of less than 5·10⁻⁶ or 2·10⁻⁷ M and has one or more of the following features: (i) it agonizes human NK cells, (ii) it causes at least 2, 4, 8, or 20 fold greater lysis in the CD44 agonist assay (as defined herein) than the amount of lysis observed in the absence of the protein ligand, (iii) it is a CD44 activity enhancing ligand, a CD44-binding cell agonist, e.g., a CD44-binding NK-cell agonist, and (iv) it is a CD44-binding cell sensitizing agent, e.g., a CD44-binding NK-cell sensitizing agent. The protein can include one or more additional features described herein. For example, the protein can include one or more features (e.g., CDRs) of BE-B12, BE-D7, BE-H10 (aka BH10), HAE-B8, HAE-F, BE-H9, HAE-H-H10 (aka HH10), or BE-A11. A “humanized” immunoglobulin variable region is an immunoglobulin variable region that includes sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

In another aspect, the invention features a method of administering a heterologous cells to a subject. The method includes administering to the subject an effective amount of a CD44 binding ligand (e.g., a CD44 binding ligand described herein), optionally, ablating or irradiating cells of the subject, and introducing heterologous cells into the subject, wherein the heterologous cells have a different HLA type from the subject or are from a different animal species than the subject.

In another aspect, the invention provides compositions, e.g., pharmaceutical compositions, which include a pharmaceutically acceptable carrier, excipient or stabilizer, and at least one of the CD44-binding ligands (e.g., antibodies or fragments thereof) described herein. In one embodiment, the compositions, e.g., the pharmaceutical compositions, include a combination of two or more of the aforesaid CD44-binding ligands.

In another aspect, the invention features a kit that includes a CD44-binding antibody (or fragment thereof), e.g., a CD44-binding antibody (or fragment thereof) as described herein, for use alone or in combination with other therapeutic modalities, e.g., a cytotoxic or labeling agent, e.g., a cytotoxic or labeling agent as described herein, along with instructions on how to use the CD44 antibody or the combination of such agents to treat, prevent or detect cancerous lesions.

In another aspect, the protein ligand that binds to CD44 is a polypeptide that is not an immunoglobulin. For example, the polypeptide can be of variable length, e.g., 4 to 100 amino acid residues in length, preferably 5 to 75, 6 to 50, or 7 to 40 amino acid residues in length, or more preferably 8 to 30 or 10 to 25 amino acid residues in length. In some embodiments, the polypeptide includes non-standard or synthetic amino acid residues, e.g., norleucine, selenocysteine, pyrrolysine, etc. In some embodiments, the polypeptide includes cross-linking groups, e.g., two cysteine residues that can form a disulfide bond or some other type of chemical cross-linking moieties that can be used to cyclize the peptide. In other preferred embodiments, the polypeptide can be modified, e.g., using polyethylene glycol or fusion to a soluble protein, so as to increase the solubility of the polypeptide.

In another aspect, the invention features a method of identifying a protein that specifically binds to CD44. For example, the method includes: providing a CD44 antigen; providing a display library (e.g., a phage display library member); identifying a member present in the library, wherein the member expresses a protein that specifically binds to the CD44 antigen. In preferred embodiments, the CD44 antigen is of human origin and includes, e.g., the extracellular domain of human CD44 or some fragment thereof, e.g., the HA binding domain of CD44. The CD44 antigen can be a recombinant polypeptide optionally fused to another polypeptide, e.g., CD44Fc, or it can be a cell that expresses CD44 (e.g., lymphocytes, particularly activated lymphocytes, or cancerous cells) on its surface. In other preferred embodiments, the CD44 antigen has an activated conformation. For example, a CD44 polypeptide antigen can be deglycosylated (e.g., by treating the polypeptide with a glycosidase, e.g., V. cholerae neuraminidase or any other glycosidase that acts upon CD44 so as to render an active conformation) or, alternatively, expressed in a cell line that produces CD44 in an activated conformation, e.g., an activated lymphocyte cell line. A CD44 antigen consisting of cells (e.g., live cells or fixed cells) that express CD44 will preferentially express an activated form of CD44, e.g., activated lymphocytes.

The methods described here are, for example, applicable to libraries that are based on bacteriophage with a substantially complete genome (e.g., including a modified gene III) and to libraries that are based on bacteriophage particles that include a phagemid nucleic acid. The terms “bacteriophage library member” and “phage” encompass members of both types of libraries. The term “bacteriophage particle” refers to a particle formed of bacteriophage coat proteins that packages a nucleic acid. The packaged nucleic acid can be a modified bacteriophage genome or a phagemid, e.g., a nucleic acid that includes a bacteriophage origin of replication but lacks essential phage genes and cannot propagate in E. coli without help from “helper phage” or phage genes supplied in trans.

In other embodiments, the invention features a method of identifying a protein that specifically binds to CD44. The method includes: providing a CD44 antigen; immunizing a mouse with the CD44 antigen; producing hybridoma cells from the spleen of the immunized mouse; and identifying individual hybridoma cell lines expressing an antibody that specifically binds to the CD44 antigen.

In preferred embodiments, the CD44 antigen is of human origin and includes, e.g., the extracellular domain of human CD44 or some fragment thereof, e.g., the HA binding domain of CD44. The CD44 antigen can be a recombinant polypeptide optionally fused to another polypeptide, e.g., CD44Fc, or it can be a cell that expresses CD44 (e.g., lymphocytes, particularly activated lymphocytes, or cancerous cells) on its surface. In other preferred embodiments, the CD44 antigen has an activated conformation. For example, a CD44 polypeptide antigen can be deglycosylated (e.g., by treating the polypeptide with a glycosidase, e.g., V. cholerae neuraminidase or any other glycosidase that acts upon CD44 so as to render an active conformation) or, alternatively, expressed in a cell line that produces CD44 in an activated conformation, e.g., an activated lymphocyte cell line. A CD44 antigen consisting of cells (e.g., live cells or fixed cells) that express CD44 will preferentially express an activated form of CD44, e.g., activated lymphocytes.

In preferred embodiments, the methods further include isolating a nucleic acid molecule from the identified phage or hybridoma, wherein the nucleic acid molecule encodes the polypeptide or antibody that specifically binds to the CD44 antigen. The isolated nucleic acid molecules can be used to produce therapeutic agents, as described herein.

In another aspect, the invention features nucleic acids that encode proteins identified by the methods described herein. In preferred embodiments, the nucleic acids include sequences encoding a heavy and light chain immunoglobulin or immunoglobulin fragment described herein. For example, the invention features a first and second nucleic acid encoding a heavy and light chain variable region, respectively, of a CD44-binding antibody molecule as described herein. Sequences encoding a heavy and light chain that function together can be present on separate nucleic acid molecules or on the same nucleic acid molecule. In another aspect, the invention features host cells and vectors containing a nucleic acid described herein or encoding a protein described herein.

In yet another aspect, the invention features a method of producing a CD44-binding antibody, or antigen-binding fragment thereof. The method includes: providing a host cell that contains a first nucleic acid encoding a polypeptide comprising a heavy chain variable region, e.g., a heavy chain variable region as described herein; providing a second nucleic acid encoding a polypeptide comprising a light chain variable region, e.g., a light chain variable region as described herein; and expressing said first and second nucleic acids in the host cell under conditions that allow assembly of said light and heavy chain variable regions to form an antigen binding protein that interacts with CD44. The first and second nucleic acids can be linked or unlinked, e.g., expressed on the same or different vector, respectively. The first and second nucleic acids can be components of the same molecule or can reside on different molecules (e.g., different chromosomes or plasmids).

The host cell can be a eukaryotic cell, e.g., a mammalian cell, an insect cell, a yeast cell, or a prokaryotic cell, e.g., E. Coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. For example, nucleic acids encoding the antibodies described herein can be expressed in a transgenic animal. In one embodiment, the nucleic acids are placed under the control of a tissue-specific promoter (e.g., a mammary specific promoter) and the antibody is produced in the transgenic animal. For example, the antibody molecule is secreted into the milk of the transgenic animal, such as a transgenic cow, pig, horse, sheep, goat or rodent. To produce a single chain antibody, the nucleic acid is configured to encode a single polypeptide that comprises both the heavy and light chain variable domains.

In another aspect, the invention features a method of inhibiting the adhesion, migration or extravasation of a cell, e.g., an activated white blood cell (e.g., an B cell, T cell, macrophage, or neutrophil), parenchymal cell, or benign or hyperplastic cell (e.g., a cell found in laryngeal, epidermal, pulmonary, breast, renal, urothelial, colonic, prostatic, or hepatic cancer and/or metastasis). The method can include contacting the cell with a CD44-binding ligand, in an amount sufficient to inhibit the adhesion, migration, or extravasation of the cell. Methods of the invention can be used, for example, to treat or prevent a disorder, e.g., an inflammatory disorder (e.g., rheumatoid arthritis, lupus, restenosis, graft v. host response, or multiple sclerosis), or a cancerous disorder (e.g., a malignant or metastatic disorder), by administering to a subject (e.g., an experimental animal or a human patient) a CD44-binding ligand in an amount effective to treat or prevent such disorder.

In another aspect, the invention features a method of contacting a cell (in vitro, ex vivo, or in vivo), e.g., an activated white blood cell (e.g., an B cell, T cell, macrophage, or neutrophil), parenchymal cell, or benign or hyperplastic cell (e.g., a cell found in laryngeal, epidermal, pulmonary, breast, renal, urothelial, colonic, prostatic, or hepatic cancer and/or metastasis). The method can include providing a ligand that interacts with CD44, e.g., a ligand described herein, and contacting the cell with the ligand, in an amount sufficient to form at least one detectable ligand-cell complex. The ligand can include, for example, a label or cytotoxic entity, e.g., an immunoglobulin Fc domain or a cytotoxic drug.

In another aspect, the invention features a method of treating, e.g., inhibiting or killing, a cell. The method includes providing a CD44-binding ligand, e.g. a ligand described herein, and contacting the cell with the ligand, in an amount sufficient to ablate or kill the cell. The contacting can be in vitro or in vivo. For example, the cell can be an activated white blood cell (e.g., an B cell, T cell, macrophage, or neutrophil), parenchymal cell, or benign or hyperplastic cell (e.g., a cell found in laryngeal, epidermal, pulmonary, breast, renal, urothelial, colonic, prostatic, or hepatic cancer and/or metastasis). The ligand can include a cytotoxic entity. The method can be used, for example, to treat or prevent a disorder, e.g., an inflammatory disorder (e.g., rheumatoid arthritis, lupus, restenosis, graft v. host response, or multiple sclerosis), or a cancerous disorder (e.g., a malignant or metastatic disorder), by administering to a subject (e.g., an experimental animal or a human patient) a CD44-binding ligand in an amount effective to treat or prevent such disorder.

The subject methods can be used on cells in culture, e.g. in vitro or ex vivo. For example, white blood cells (e.g., B cells, T cells, macrophages, or neutrophils), parenchymal cells, or cancerous or metastatic cells (e.g., laryngeal, epidermal, pulmonary, breast, renal, urothelial, colonic, prostatic, or hepatic cancer or metastatic cells) can be cultured in vitro in culture medium and the contacting step can be effected by adding the CD44-binding ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the CD44-binding ligand to the subject under conditions effective to permit both binding of the ligand to the cell, and the inhibition of adhesion, migration, or extravasation of the cell, or the treating, e.g., the killing or ablating, of the cell.

The method can be used to treat or prevent cancerous disorders, e.g., including but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, as well as adenocarcinomas which include malignancies such as most colon cancers, rectal cancer, renal-cell carcinoma, liver cancer, non-small cell carcinoma of the lung, cancer of the small intestine and cancer of the esophagus. In particular, metastatic lesions of the aforementioned cancers can also be treated or prevented using the methods or compositions described herein.

The subject can be a mammal, e.g., a primate, preferably a higher primate, e.g., a human (e.g., a patient having, or at risk of, a disorder described herein, e.g., cancer).

The CD44-binding antibody or fragment thereof, e.g., a CD44-binding antibody or fragment thereof as described herein, can be administered to the subject systemically (e.g., orally, parenterally, subcutaneously, intravenously, intramuscularly, intraperitoneally, intranasally, transdermally, or by inhalation), topically, or by application to mucous membranes, such as the nose, throat and bronchial tubes.

The methods can further include the step of monitoring the subject, e.g., for a reduction in one or more of: a reduction in tumor size; reduction in cancer markers, e.g., levels of cancer specific antigen; reduction in the appearance of new lesions, e.g., in a bone scan; a reduction in the appearance of new disease-related symptoms; or decreased or stabilization of size of soft tissue mass; or any parameter related to improvement in clinical outcome. The subject can be monitored in one or more of the following periods: prior to beginning of treatment; during the treatment; or after one or more elements of the treatment have been administered. Monitoring can be used to evaluate the need for further treatment with the same CD44-binding ligand or for additional treatment with additional agents. Generally, a decrease in one or more of the parameters described above is indicative of the improved condition of the subject.

The CD44-binding ligand can be used alone in unconjugated form to thereby inhibit adhesion, migration, or extravasation or the CD44-expressing cells, or ablate or kill the CD44-expressing cells. If the ligand is an antibody, the ablation or killing can be mediated, e.g., by an antibody-dependent cell killing mechanisms such as complement-mediated cell lysis and/or effector cell-mediated cell killing. In other embodiments, the CD44-binding ligand can be bound to a substance, e.g., a cytotoxic agent or moiety, effective to kill or ablate the CD44-expressing cells. For example, the CD44-binding ligand can be coupled to a radioactive ion (e.g., an α-, γ-, or β-emitter), e.g., iodine (¹³¹I or ¹²⁵I), yttrium (⁹⁰Y), lutetium (¹⁷⁷Lu), actinium (²²⁵Ac), or bismuth (²¹³Bi). The methods and compositions described herein can be used in combination with other therapeutic modalities. In one embodiment, the method includes administering to the subject a CD44-binding ligand, e.g., a CD44-binding antibody or fragment thereof, in combination with a cytotoxic agent, in an amount effective to treat or prevent said disorder. The ligand and the cytotoxic agent can be administered simultaneously or sequentially. In other embodiments, the methods and compositions described herein are used in combination with surgical and/or radiation procedures.

In another aspect, the invention features methods for detecting the presence of a CD44 protein, in a sample, in vitro (e.g., a biological sample, a tissue biopsy, e.g., a cancerous lesion). The subject method can be used to evaluate, e.g., diagnose or stage a disorder described herein, e.g., a cancerous disorder. The method includes: (i) contacting the sample (and optionally, a reference, e.g., control, sample) with a CD44-binding ligand, as described herein, under conditions that allow interaction of the CD44-binding ligand and the CD44 protein to occur; and (ii) detecting formation of a complex between the CD44-binding ligand, and the sample (and optionally, the reference, e.g., control, sample). Formation of the complex is indicative of the presence of CD44 protein (e.g., activated CD44 protein), and can indicate the suitability or need for a treatment described herein. For example, a statistically significant change in the formation of the complex in the sample relative to the reference sample, e.g., the control sample, is indicative of the presence of CD44 (e.g., activated CD44) in the sample.

In yet another aspect, the invention provides a method for detecting the presence of CD44 (e.g., activated CD44) in vivo (e.g., in vivo imaging in a subject). The subject method can be used to evaluate, e.g., diagnose, localize, or stage a disorder described herein, e.g., a cancerous disorder. The method includes: (i) administering to a subject (and optionally a control subject) a CD44-binding ligand (e.g., an antibody or antigen binding fragment thereof), under conditions that allow interaction of the CD44-binding ligand and the CD44 protein to occur; and (ii) detecting formation of a complex between the ligand and CD44, wherein a statistically significant change in the formation of the complex in the subject relative to the reference, e.g., the control subject or subject's baseline, is indicative of the presence of the CD44. The presence of activated CD44 in particular locations within a subject (e.g., in the joints or in close proximity to a solid tumor) can be indicative of an inflammatory disorder or cancer, e.g., metastatic cancer.

In other embodiments, a method of diagnosing or staging, a disorder as described herein (e.g., an inflammatory or cancerous disorder), is provided. The method includes: (i) identifying a subject having, or at risk of having, the disorder; (ii) obtaining a sample of a tissue or cell affected with the disorder; (iii) contacting said sample or a control sample with a CD44-binding ligand, under conditions that allow interaction of the binding agent and the CD44 protein to occur, and (iv) detecting formation of a complex. A statistically significant increase in the formation of the complex between the ligand with respect to a reference sample, e.g., a control sample, is indicative of the disorder or the stage of the disorder. For example, the finding of activated CD44 on tumor cells located in a solid tumor can indicate that the tumor is progressing into a metastatic tumor.

Preferably, the CD44-binding ligand used in the in vivo and in vitro diagnostic methods is directly or indirectly labeled with a detectable substance to facilitate detection of the bound or unbound binding agent. Suitable detectable substances include various enzymes, prosthetic groups, fluorescent materials, luminescent materials and radioactive materials. In one embodiment, the CD44-binding ligand is coupled to a radioactive ion, e.g., indium (¹¹¹In), iodine (¹³¹I or ¹²⁵I), yttrium (⁹⁰Y), actinium (²²⁵Ac), bismuth (²¹³Bi), sulfur (³⁵S), carbon (¹⁴C), tritium (³H), rhodium (¹⁸⁸Rh), or phosphorous (³²p). In another embodiment, the ligand is labeled with an NMR contrast agent.

In another aspect, the invention provides a ligand (e.g., a ligand that includes one or more immunoglobulin variable domains, e.g., domains that form an immunoglobulin antigen binding site) that interacts with CD44 and which functions as a CD44 agonist. For example, interaction between the ligand and an NK cell can sensitize the NK cell to radiation or increase CD44 protein levels on the cell. Exemplary ligands may include BE-B12, BE-D7, BE-H10(aka BH10), HAE-B8, HAE-F1, BE-H9, HAE-H-H10 (aka HH10), and BE-A11, and related antibodies.

In one embodiment, the ligand competes with the monoclonal S5 which binds to canine CD44 for binding to a CD44, e.g., to a human or canine CD44. (See, e.g., Sandmaier et al. (1998) Blood 91:3494-3502). In one embodiment, the ligand binds to an epitope that overlaps with an epitope bound by monoclonal S5. The invention also includes methods of preparing and administering such ligands, e.g., in an amount effective to aid a subject (e.g., to increase the engraftment rate of bone marrow). For example, the ligand can be administered at a dose of between 0.05 to 10 mg/kg/day, e.g., 0.05 to 2 mg/kg/day.

In another aspect, the invention provides a ligand (e.g., a ligand that includes one or more immunoglobulin variable domains, e.g., domains that form an immunoglobulin antigen binding site) that interacts with CD44 and that increases the engraftment rate of a HLA-nonidentical bone marrow. In one embodiment, the ligand does not elicit a substantial (e.g., a detectable or an adverse) immune response in a human subject. In one embodiment, the ligand is a CD44 activity enhancing ligand, a CD44-binding cell agonist, e.g., a CD44-binding NK-cell agonist, or a CD44-binding cell sensitizing agent, e.g., a CD44-binding NK-cell sensitizing agent.

In another aspect, the invention provides a protein that competes with the monoclonal S5 for binding to a CD44, e.g., to a human or canine CD44. (See, e.g., Sandmaier et al. (1998) Blood 91:3494-3502). For example, the protein includes one or more immunoglobulin variable domains, e.g., domains that form an immunoglobulin antigen binding site, e.g., domains with human framework regions, e.g., one, two, three, four, five, six, seven or eight human framework regions. The protein can include one or more human CDRs, e.g., one, two, three, four, five, or six human CDRs. For example, the LC CDRs can be human. In another example, HC CDR3 is human. In one embodiment, the antibody includes at least three human CDRs. The antibody can also include one or two synthetic CDRs.

In one embodiment, one or more the CDRs of the immunoglobulin variable domains differs from a respective CDR of the S5 monoclonal by at least one amino acid, e.g., at least one, two, three, four, five, or seven. For example, each CDR differs from a respective CDR of the S5 monoclonal by at least one, two, or three amino acids. In one embodiment, the protein binds to human CD44 with a K_{d} of less than 10⁻⁶, 10⁻⁷, 10⁻⁸, 10⁻⁹, or 10⁻¹⁰ M. In one embodiment, the protein induces lysis of NK cells in vitro, e.g., at least 2, 3, 4, 5, 10, or 20 more % lysis relative to a parallel control, or, e.g., at least 15, 20, 35, 40, 50, 60, 65, 70, or 85% lysis.

Certain CD44 agonists may increase CD44 binding affinity for HA, e.g., at least 0.2, 0.5, 0.7, 1.0, 1.2, 1.5, 2.0, 4, 5, or 10 fold increase, e.g., using a cell-free or cell-based assay.

In another aspect, the invention provides a method of grafting cells into a subject. The method includes administering to the subject a ligand described herein, e.g., a CD44 agonist, and then grafting cells (e.g., bone marrow cells) into the subject. The method can also include killing, impairing, or attenuating hematopoietic cells in the subject. For example, the method can include irradiating the subject or cells of the subject, e.g., to kill NK cells in the subject. The ligand can be administered in an amount effective to sensitize NK cells in the subject to the killing, impairing, or attenuating, e.g., to irradiation. For example, the ligand can be administered in an amount of between 0.05 to 10 mg/kg/day, e.g., 0.05 to 2 mg/kg/day. In one embodiment, the subject is human and ligand includes an immunoglobulin antigen binding site formed by immunoglobulin variable domains, e.g., with human framework regions. In one embodiment, HLA type of the grafted cells is non-identical to the HLA type of the subject.

The invention also provides polypeptides and nucleic acids that encompass a range of amino acid and nucleic acid sequences. In addition, the invention features a host cell that includes a nucleic acid described herein. The cell can express a protein described herein, e.g., on its surface.

As used herein, the term “antibody” refers to a protein comprising at least one immunoglobulin variable domain. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is composed of three CDR's and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may omit one, two or more N- or C-terminal amino acids, or may include other alterations.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region includes three domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof), wherein the light chains of the immunoglobulin may be of types kappa or lambda. In one embodiment, the antibody is glycosylated. An antibody can be functional for antibody-dependent cytotoxicity and/or complement-mediated cytotoxicity.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH—-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody (or simply “antibody portion,” or “fragment”), as used herein, refers to one or more fragments of a full-length antibody that retain the ability to specifically bind to CD44 (e.g., human CD44). Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883. The term “antibody” encompasses antigen-binding fragments of antibodies (e.g., single chain antibodies, Fab fragments, F(ab′)₂, a Fd fragment, a Fv fragments, and dAb fragments) as well as complete antibodies. Antibody fragments can be obtained using any appropriate technique including conventional techniques known to those with skill in the art.

As used herein, “activated” CD44 refers to the predominant form of CD44 present on activated white blood cells. As used herein, “resting” CD44 or CD44 that is “not activated” refers to the predominant form of CD44 present on resting white blood cells. White blood cells is a term of art that encompasses, e.g., lymphocytes (e.g., B- and T-cells and their precursors), monocytes, and neutrophils. Functionally, activated CD44 can be distinguished from non-activated forms of CD44 by its higher (e.g., 10%, 20%, 30%, 40%, 50%, 60%, 70%, 80%, 90%, 100%, 500%, 1000%, or higher) affinity for hyaluronan (HA). Activated CD44 can be distinguished from non-activated CD44 on the basis of a structural property, e.g., glycosylation (e.g., reduced glycosylation), additional polypeptide sequences resulting from alternative splicing (e.g., inclusion of exons v4-v7), conformational changes induced by protein-protein interactions (e.g., the clustering of CD44 on the cell surface), or some combination thereof. The term “CD44 ectodomain” refers to any extracellular region of a CD44 protein.

As used herein, “deglycosylated” CD44 refers to CD44 proteins exhibiting a decrease in glycosylation as compared to CD44 present on resting white blood cells. Decreases in CD44 glycosylation can be cell specific, e.g., cell-type specific (a function of the cell type in which the CD44 protein is expressed (e.g., a metastatic tumor cell)), or cell-state specific (a function of the state of the cell in which the CD44 protein is expressed (e.g., an activated lymphocyte)). The degree of glycosylation of CD44 can be determined by its mobility, e.g., following electrophoresis, or by assaying for particular glycosylation epitopes, e.g., by ELISA. The deglycosylated form may include at least one, two, four, or six or more amino acids that lack a glycosyl modification at positions that are glycosylated in a CD44 molecule in a resting cell.

As used herein, “high-affinity” CD44 refers to CD44 that has a higher affinity for HA as compared to CD44 present on resting white blood cells. The higher affinity can be a reflection of molecular changes in the protein, e.g., arising from alternative splicing or changes in glycosylation, it can be a reflection of the cell in which the CD44 is expressed (e.g., an activated white blood cell or a metastatic cell), or both.

As used herein, “activation” used with respect to white blood cells refers to cellular changes triggered by T cell receptor and/or cytokine stimulation associated with their recruitment to a site of, or response to, inflammation.

As used herein, “binding affinity” refers to the apparent association constant or Ka. The Ka is the reciprocal of the dissociation constant (Kd). A ligand may, for example, have a binding affinity of at least 10⁻5, 10⁻⁶, 10⁻⁷ or 10⁻⁸ M for a particular target molecule. Higher affinity binding of a ligand to a first target relative to a second target can be indicated by a higher Ka (or a smaller numerical value Kd) for binding the first target than the Ka (or numerical value Kd ) for binding the second target. In such cases the ligand has specificity for the first target relative to the second target.

Binding affinity can be determined by a variety of methods including equilibrium dialysis, equilibrium binding, gel filtration, ELISA, surface plasmon resonance, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:

[Bound]=N·[Free]/((1/Ka)+[Free]).

It is not always necessary to make an exact determination of Ka, though, since sometimes it is sufficient to obtain a quantitative measurement of affinity, e.g., determined using a method such as ELISA or FACS analysis, is proportional to Ka, and thus can be used for comparisons, such as determining whether a higher affinity is, e.g., 2-fold higher. Exemplary conditions for evaluating binding affinity are in PBS (phosphate buffered saline) at pH 7.2 at 30° C.

An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 92, 95, 98, or 99% pure on a weight-weight basis.

An “epitope” refers to the site on a target compound that is bound by a ligand, e.g., a polypeptide ligand or an antigen-binding ligand (e.g., an antibody such as a Fab or full length antibody). In the case where the target compound is a protein, the site can be entirely composed of amino acid components, entirely composed of chemical modifications of amino acids of the protein (e.g., glycosyl moieties), or composed of combinations thereof. Overlapping epitopes include at least one common amino acid residue.

A “CD44 activity enhancing ligand” is a ligand that increase CD44 affinity for HA by at least 10%. For example, some CD44-binding antibodies may increase CD44 affinity for HA. For example, a CD44-binding antibody may increase affinity of an interaction between by at least 10, 15, 20, 30, 50, 75, 100, or 110%, e.g., when present at a concentration of between 0.1 μg/ml to 10 μg/ml.

A “CD44-binding cell agonist” is a CD44-binding protein that activates a CD44-expressing cells as determined by an assay described herein. A “CD44-binding cell sensitizing agent” is a CD44-binding molecule that can reduce the amount of total body irradiation required to kill a CD44-expressing cells in a subject. A “CD44-binding NK-cell agonist” is a CD44-binding protein that activates NK cells as determined by an assay described herein. A “CD44-binding NK-cell sensitizing agent” is a CD44-binding molecule that can reduce the amount of total body irradiation required to kill NK cells in a subject.

A “CD44 antagonist” refers to a CD44 interacting molecule that reduces the ability of CD44 to bind to HA. For example, the antagonist may reduce the affinity of CD44 to HA, e.g., by reducing the Ka at least 20, 40, 50, 60, 70, 80, 90, or 95%.

A “S5-like antibody” refers to an antibody that competes with S5 for binding to canine CD44 or an antibody that binds to a corresponding epitope of human CD44. A “S5-like engraftment enhancing antibody” refers to an S5-like antibody that enhances hematopoietic cell engraftment in a canine model or a human subject.

“An immunosuppressive agent capable of inactivating thymic or lymph node T cells”, as used herein, is an agent other than an antibody, e.g., a chemical agent, e.g., a drug, that, when administered at an appropriate dosage, results in the inactivation of thymic or lymph node T cells. Examples of such agents are cyclosporine, FK-506, and rapamycin. Such agents can be used in conjunction with a CD44-binding NK-cell agonist, e.g., prior to or after transfer of exogenous cells.

“Tolerance”, as used herein, refers to the inhibition of a graft recipient's immune response which would otherwise occur, e.g., in response to the introduction of a nonself MHC antigen into the recipient. Tolerance can involve humoral, cellular, or both humoral and cellular responses.

“Hematopoietic stem cell”, as used herein, refers to a cell, e.g., a bone marrow cell which is capable of developing into a mature myeloid and/or lymphoid cell. Stem cells derived from the cord blood of the recipient or the donor can be used in certain implementations, e.g., as a source of exogenous cells for transfer.

“Graft”, as used herein, refers to a body part, organ, tissue, or cells. Grafts may consist of organs such as liver, kidney, heart or lung; body parts such as bone or skeletal matrix; tissue such as skin, intestines, endocrine glands; or progenitor stem cells of various types.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity relative to the same antigen.

Sequences similar or homologous (e.g., at least about 85% sequence identity) to the sequences disclosed herein are also part of this application. In some embodiment, the sequence identity can be about 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

As used herein, the term “homologous” is synonymous with “similarity” and means that a sequence of interest differs from a reference sequence by the presence of one or more amino acid substitutions (although modest amino acid insertions or deletions) may also be present. In addition to the GAP program described above, a variety of means of calculating degrees of homology or similarity to a reference sequence are available. One method uses the BLAST algorithms (available from the National Center of Biotechnology Information (NCBI), National Institutes of Health, Bethesda Md.), in each case, using the algorithm default or recommended parameters for determining significance of calculated sequence relatedness. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5M sodium phosphate, 7% SDS at 65° C., followed by one or more washes at 0.2× SSC, 1% SDS at 65° C. Very high stringency conditions (4) are the preferred conditions and the ones that should be used unless otherwise specified. The invention includes nucleic acids that hybridize with low, medium, high, or very high stringency to a nucleic acid described herein or to a complement thereof. The nucleic acids can be the same length or within 30, 20, or 10% of the length of the reference nucleic acid.

It is understood that a CD44-binding ligand may have mutations relative to a CD-binding ligand described herein (e.g., a conservative or non-essential amino acid substitutions), which do not have a substantial effect on the polypeptide functions. Whether or not a particular substitution will be tolerated, i.e., will not adversely affect desired biological properties, such as binding activity can be determined as described in Bowie, et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). It is possible for many framework and CDR amino acid residues to include one or more conservative substitutions.

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

The terms “polypeptide” or “peptide” (which may be used interchangeably) refer to a polymer of three or more amino acids linked by a peptide bond, e.g., between 3 and 30, 12 and 60, or 30 and 300, or over 300 amino acids in length. The polypeptide may include one or more unnatural amino acids. Typically, the polypeptide includes only natural amino acids. A “protein” can include one or more polypeptide chains. Accordingly, the term “protein” encompasses polypeptides. A protein or polypeptide can also include one or more modifications, e.g., a glycosylation, amidation, phosphorylation, and so forth. The term “small peptide” can be used to describe a polypeptide that is between 3 and 30 amino acids in length, e.g., between 8 and 24 amino acids in length. The term “ligand” refers to a protein that can interact, e.g., specifically interact, with a target molecule, e.g., CD44.

Other features and advantages of the instant invention will become more apparent from the following detailed description and claims. Embodiments of the invention can include any combination of features described herein. The contents of all references, pending patent applications and published patents, cited throughout this application are hereby expressly incorporated by reference, inclusive of U.S. Ser. No. 60/410,758, filed Sep. 13, 2002 and Ser. No. 60/469,123, filed May 9, 2003.

### DETAILED DESCRIPTION

CD44 is a cell surface protein. It is accessible and susceptible to targeting by the antibodies and other ligands described herein. CD44 participates, e.g., in adhesion, migration, and extravasation by certain CD44-expressing cells.

The invention provides, inter alia, proteins that bind to CD44, e.g., the extracellular region of mature CD44. In one embodiment, the protein is a CD44 antagonist. In another embodiment, the protein is a CD44 activity enhancing ligand, a CD44 binding cell agonist, or a CD44-binding cell sensitizing agent.

CD44-binding proteins can be used to modulate a CD44-expressing cell or a function of CD44, e.g., cell adhesion, migration, or extravasation. For example, a CD44-binding protein can be used to treat diseases, e.g., particularly diseases in which CD44-expressing cells contribute to pathology, e.g., inflammatory diseases and cancer. The recruitment of lymphocytes to sites of inflammation can be inhibited by blocking the interaction between activated CD44 and HA. Likewise, blocking the interaction between CD44 and HA can inhibit the metastasis of tumor cells that depend upon the CD44/HA interaction for cellular migration and/or extravasation from the vascular system.

In one embodiment, the CD44-binding protein is an antibody, e.g., a full length-antibody, or an antigen-binding fragment of a full length antibody. In another embodiment, the protein is a modified scaffold polypeptide (or peptide). In still another preferred embodiment, the protein ligand is a cyclic peptide or a linear peptide, e.g., of less than 30 or 25 amino acids. Whereas some examples described herein refer to antibody ligands or fragments thereof, it is understood, that the invention can be practiced using any protein ligand (e.g., antibody and non-antibody ligand, e.g., a ligand having a structural fold described herein).

It is appreciated that there can be naturally occurring or artificial genetic variation in genes encoding the CD44 amino acid sequence that may result in a variety of CD44 amino acid sequences, typically at least 96%, 97%, 98%, or 99% homologous to a CD44 amino acid sequence provided herein, e.g., differing by fewer than ten, five, or three amino acid substitutions. For example, one natural variation is at position 46 of SEQ ID NO:1 (PRO-46 or ARG-46). Preferably, the CD44 amino acid sequence is a functional CD44 amino acid sequence, e.g., the sequence of an HA binding fragment of a mature, full-length CD44 protein. The protein may be functional for extravasation and/or cell migration.

Alternative splicing can lead to a variety of CD44 amino acid sequences. Screaton et al. (1992), Proc. Natl. Acad. Sci. USA 89:12160-4. Exemplary CD44 amino acid sequences area as follows:

| | |
|---|---|
| CD44 Isoform: Exons 1-17 and 19: | |
| MDKFWWHAAWGLCLVPLSLAQIDLNITCRFAGVFHVEKNGRYSISRTEAADLCKAFNSTL | (SEQ ID NO:1) |
| PTMAQMEKALSIGFETCRYGFIEGHVVIPRIHPNSICAANNTGVYILTSNTSQYDTYCFN | |
| ASAPPEEDCTSVTDLPNAFDGPITITIVNRDGTRYVQKGEYRTNPEDIYPSNPTDDDVSS | |
| GSSSERSSTSGGYIFYTFSTVHPIPDEDSPWITDSTDRIPATTLMSTSATATETATKRQE | |
| TWDWFSWLFLPSESKNHLHTTTQMAGTSSNTISAGWEPNEENEDERDRHLSFSGSGIDDD | |
| EDFISSTISTTPRAFDHTKQNQDWTQWNPSHSNPEVLLQTTTRMTDVDRNGTTAYEGNWN | |
| PEAHPPLIHHEHHEEEETPHSTSTIQATPSSTTEETATQKEQWFGNRWHEGYRQTPREDS | |
| HSTTGTAAASAHTSHPMQGRTTPSPEDSSWTDFFNPISHPMGRGHQAGRRMDMDSSHSTT | |
| LQPTANPNTGLVEDLDRTGPLSMTTQQSNSQSFSTSHEGLEEDKDHPTTSTLTSSNRNDV | |
| TGGRRDPNHSEGSTTLLEGYTSHYPHTKESRTFIPVTSAKTGSFGVTAVTVGDSNSNVNR | |
| SLSGDQDTFHPSGGSHTTHGSESDGHSHGSQEGGANTTSGPIRTPQIPEWLIILASLLAL | |
| ALILAVCIAVNSRRRCGQKKKLVINSGNGAVEDRKPSGLNGEASKSQEMVHLVNKESSET | |
| PDQFMTADETRNLQNVDMKIGV | |
| Exons 1-5, 12-17 and 19: | |
| MDKFWWHAAWGLCLVPLSLAQIDLNITCRFAGVFHVEKNGRYSISRTEAADLCKAFNSTL | (SEQ ID NO:2) |
| PTMAQMEKALSIGFETCRYGFIEGHVVIPRIGPNSICAANNTGVYILTSNTSQYDTYCFN | |
| ASAPPEEDCRSVTDLPNAFDGPITITIVNRDGTRYVQKGEYRTNPEDIYPSNPTDDDVSS | |
| GSSSERSSTSGGYIFYTFSTVHPIPDEDSPWITDSTDRIPATNMDSSHSTTLQPTANPNT | |
| GLVEDLDRTGPLSMTTQQSNSQSFSTSHEGLEEDKDHPTTSTLTSSNRNDVTGGRRDPNH | |
| SEGSTTLLEGYTSHYPHTKESRTFIPVTSAKTGSFGVTAVTVGDSNSNVNRSLSGDQDTF | |
| HPSGGSHTTHGSESDGHSHGSQEGGANTTSGPIRTPQIPEWLIILASLLALALILAVCIA | |
| VNSRRRCGQKKKLVINSGNGAVEDRKPSGLNGEASKSQEMVHLVNKESSETPDQFMTADE | |
| TRNLQNVDMKIGV | |
| Exons 1-5, 15-17 and 19: | |
| MDKFWWHAAWGLCLVPLSLAQIDLNITCRFAGVFHVEKNGRYSISRTEAADLCKAFNSTL | (SEQ ID NO:3) |
| PTMAQMEKALSIGFETCRYGFIEGHVVIPRIHPNSICAANNTGVYILTSNTSQYDTYCFN | |
| ASAPPEEDCRSVTDLPNAFDGPITITIVNRDGTRYVQKGEYRTNPEDIYPSNPEDDDVSS | |
| GSSSERSSTSGGYIFYTFSTVHPIPDEDSPWITDSTDRIPATRDQDTFHPSGGSHTTHGS | |
| ESDGHSHGSQEGGANTTSGPIRTPQIPEWLIILASLLALALILAVCIAVNSRRRCGQKKK | |
| LVINSGNGAVEDRKPIGLNGEASKSQEMVHLVNKESSETPDQFMTADETRNLQNVDMKIG | |
| V | |

In one embodiment, the protein ligands can bind to an epitope present in at least one of the amino acid sequences selected from the group consisting of SEQ ID NO:1, SEQ ID NO:2, and SEQ ID NO:3, including epitopes arising from the glycosylation of such sequences.

Exemplary epitopes include the IM7 binding site and the S5 monoclonal binding site. Epitopes can include one or more amino acids in the sequence: DLPNAFDGPITIT, e.g., the sequence at about amino acid residue 134 to 147 of SEQ ID NO:1. For example, the epitope can include one or more amino acids between 22-150, 22-75, 76-150, 130-200, 140-190, 145-185, or 150-300 of SEQ ID NO:1. The epitope may include determinants that are N-terminal of amino acid 400, 300, 250, 200, or 150 of SEQ ID NO:1.

An exemplary mature CD44 amino acid sequence is 21-742 of SEQ ID NO:1. An exemplary extracellular domain is 21-649 of SEQ ID NO:1. Exemplary glycosylation sites include amino acid positions 25, 57, 100, 120, 548, 599, and 636 of SEQ ID NO:1. In some CD44 variants, these positions are not glycosylated, either alone or in combination.

The CD44-binding proteins bind to human CD44 with high affinity and specificity, and thus can be used as diagnostic, prophylactic, or therapeutic agents in vivo and in vitro. Preferably the ligands specifically bind to the CD44. As used herein, “specific binding” refers to the property of the antibody: (1) to bind to CD44, e.g., human CD44, with an affinity of at least 2×10⁷ M⁻, and (2) to preferentially bind to CD44, e.g., human CD44, with an affinity that is at least two-fold, 50-fold, 100-fold, or more, greater than its affinity for binding to a non-specific antigen (e.g., BSA, casein) other than CD44. (The terms “CD44-binding ligands” and “CD44 ligands” are used interchangeably to refer to ligands that can interact with CD44).

CD44 Agonists

CD44 agonists can be used to modulate immune cells in a subject. For example, CD44 agonists can be used to sensitize NK cells. The sensitized cells can then be ablated, e.g., using radiation, a cytotoxin directed against the cells, or other drug that triggers ablation. Without intending to be bound by theory, a CD44 agonist can be used to increase CD44 protein levels on target cells or CD44 activity. See, e.g., Sandmaier et al. (1998) Blood 91:3494-3502). Modulation of an immune system with a CD44 ligand can be appropriate prior, during, or after introduction of antigenic material into a subject, e.g., introducing a HLA-nonidentical cell or tissue, e.g., bone marrow or prior, during, or after treatment of graft-versus-host disease (GVHD). Thus, treatment with a CD44 agonist can be used, e.g., before a transplant, e.g., an organ or bone marrow transplant. Bone marrow transplantation can be used to treat a wide variety of diseases, including hematological malignancies, aplastic anemia, red cell disorders and congenital immunodeficiencies.

CD44 antibodies can be evaluated for agonist activity in an NK cell assay. Sandmaier et al. (1998) Blood 91(9):3494-502. Canine peripheral blood monocytic cell (PBMC) effector populations are incubated with CD44 antibody, then aliquotted into wells of round-bottom 96-well plates containing an equal volume of medium (spontaneous release) or of 2% Triton-X (maximum release). NK-sensitive target canine thyroid adenocarcinoma cells (CTAC) are loaded with chromium-51 (5 mCi/mL of medium) in a 1 hr, 37° C., 5% CO₂ incubation, then washed three times with 10-fold excess cold medium, resuspended to 5×10⁴ to 10⁵ cells/mL, and 5×10³ to 10⁴ cells are added to the effector cells. After 16 hr incubation at 37° C., 5% CO₂, plates are centrifuged and supernatants are obtained for measurement of released radioactivity in a gamma scintillation counter. Percent specific lysis is calculated:

$$Specific lysis ( \% ) = \frac{cpm ( experimental ) - cpm ( spontaneous release )}{cpm ( maximum release ) - cpm ( spontaneous release )} \times 100.$$

The extent of specific lysis after CD44 antibody incubation compared to lysis after incubation in medium alone is an indication of CD44 antibody agonist activity. A similar assay can be done with human peripheral blood monocytic cells if the antibody is specific for humans, relative to canine CD44. It is appreciated that the ability of a CD44 antibody to be a CD44 activity enhancing ligand, a CD44-binding cell agonist, e.g., a CD44-binding NK-cell agonist, or a CD44-binding cell sensitizing agent, e.g., a CD44-binding NK-cell sensitizing agent, may be empirical and may not require a particular result in a particular assay to be useful as such.

Identifying a CD44-Binding Ligand

In one implementation, proteins that bind to CD44, e.g., activated CD44, deglycosylated CD44, or high-affinity CD44, are identified by a method that includes: providing a library of coding nucleic acids and selecting from the library one or more members that encode a protein that binds to the CD44 antigen. The selection can be performed in a number of ways. For example, the library can be a display library. Similarly, a CD-44 binding protein can be isolated from a library of proteins, e.g., proteins on a protein array.

The CD44 can be tagged and recombinantly expressed. The CD44 is purified and attached to a support, e.g., to affinity beads, or paramagnetic beads or other magnetically responsive particles.

The CD44 can also be expressed on the surface of a cell. Members of the display library that specifically bind to the cell, e.g., only if the CD44 is activated, can be selected.

Display Libraries

In one embodiment, a display library is used to identify proteins that bind to CD44. A display library is a collection of entities; each entity includes an accessible polypeptide component and a recoverable component that encodes or identifies the polypeptide component. The polypeptide component can be of any length, e.g. from three amino acids to over 300 amino acids. In a selection, the polypeptide component of each member of the library is probed with CD44 protein and if the polypeptide component binds to CD44, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

Phage Display. One format utilizes viruses, particularly bacteriophages. This format is termed “phage display.” The polypeptide component is typically covalently linked to a bacteriophage coat protein. The linkage results form translation of a nucleic acid encoding the polypeptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chein 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage f1, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshmet al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In one embodiment, the fusion is to a domain of the gene III protein, e.g., the anchor domain or “stump,” (see, e.g., U.S. Pat. No. 5,658,727 for a description of the gene III protein anchor domain). Phagemid and other modifications of the fundamental technology are also available.

Bacteriophage displaying the polypeptide component can be grown and harvested using standard phage preparatory methods, e.g. PEG precipitation from growth media.

After selection of individual display phages, the nucleic acid encoding the selected polypeptide components, by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and WO 03/029456 which describes a yeast display system that can be used to display immunoglobulin proteins such as Fab fragments, and the use of mating to generate combinations of heavy and light chains.

In one embodiment, diverse nucleic acid sequences are cloned into a vector for yeast display. The cloning joins the diverse sequence with a domain (or complete) yeast cell surface protein, e.g., Aga2, Aga1, Flo1, or Gas1. A domain of these proteins can anchor the polypeptide encoded by the diverse nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

In one embodiment, nucleic acids encoding immunoglobulin heavy chains that have been mutagenized based on an initial CD44-binding immunoglobulin are introduced into yeast cells of one cell type, and nucleic acids encoding immunoglobulin light chains that have been mutagenized based on an initial CD44-binding immunoglobulin are introduced into yeast cells of the other cell type. These two populations of cells can be combined to form diploid yeast that each express an immunoglobulin heavy and light chain. The yeast cells can be selected and/or screened for cells that bind to CD44, e.g., bind with improved affinity.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30; and Schaffitzel et al. (1999) J Immunol Methods. 231(1-2):119-35.

Protein-Nucleic Acid Fusions. Another format utilizes protein-nucleic acid fusions. Protein-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the protein.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Scaffolds. Scaffolds for display can include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains).

Appropriate criteria for evaluating a scaffolding domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffolding domain is a small, stable protein domains, e.g., a protein of less than 100, 70, 50, 40 or 30 amino acids. The domain may include one or more disulfide bonds or may chelate a metal, e.g., zinc.

Examples of small scaffolding domains include: Kunitz domains (58 amino acids, 3 disulfide bonds), Cucurbida maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds).

Examples of small intracellular scaffolding domains include SH2, SH3, and EVH domains. Generally, any modular domain, intracellular or extracellular, can be used.

A display library of variants of a scaffold domain can be screened, e.g., to identify an epitope specific ligand that binds to CD44. For example, the epitope can be an epitope bound by a ligand described herein, e.g., the S5 antibody. In one embodiment, the scaffold domain is other than an antibody domain. An exemplary method for identifying an epitope specific ligand includes using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target. Another exemplary method includes using a ligand that binds to the epitope of interest as an eluant to elute display library members bound to CD44.

Iterative Selection. In one preferred embodiment, display library technology is used in an iterative mode. A first display library is used to identify one or more ligands for a target. These identified ligands are then varied using a mutagenesis method to form a second display library. Higher affinity ligands are then selected from the second library, e.g., by using higher stringency or more competitive binding and washing conditions.

In some implementations, the mutagenesis is targeted to regions known or likely to be at the binding interface. If, for example, the identified ligands are antibodies, then mutagenesis can be directed to the CDR regions of the heavy or light chains as described herein. Further, mutagenesis can be directed to framework regions near or adjacent to the CDRs, e.g., framework regions, particular within ten, five, or three amino acids of a CDR junction.. In the case of antibodies, mutagenesis can also be limited to one or a few of the CDRs, e.g., to make precise step-wise improvements.

Some exemplary mutagenesis techniques include: error-prone PCR (Leung et al. (1989) Technique 1:11-15), recombination (see, e.g., U.S. Serial No. 10/279,633, filed Oct. 24, 2002), DNA shuffling using random cleavage (Stemmer (1994) Nature 389-391; termed “nucleic acid shuffling”), RACHITT™ (Coco et al. (2001) Nature Biotech. 19:354), site-directed mutagenesis (Zoller et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J 13:3245).

In one example of iterative selection, the methods described herein are used to first identify a protein ligand from a display library that binds a CD44 with at least a minimal binding specificity for a target or a minimal activity, e.g., an equilibrium dissociation constant for binding of greater than 1 nM, 10 nM, or 100 nM. The nucleic acid sequence encoding the initial identified protein ligand are used as a template nucleic acid for the introduction of variations, e.g., to identify a second protein ligand that has enhanced properties (e.g., binding affinity, kinetics, or stability) relative to the initial protein ligand.

Off-Rate Selection. Since a slow dissociation rate can be predictive of high affinity, particularly with respect to interactions between polypeptides and their targets, the methods described herein can be used to isolate ligands with a desired kinetic dissociation rate (i.e. reduced) for a binding interaction to a target.

To select for slow dissociating ligands from a display library, the library is contacted to an immobilized target. The immobilized target is then washed with a first solution that removes non-specifically or weakly bound biomolecules. Then the immobilized target is eluted with a second solution that includes a saturation amount of free target, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

Selecting and Screening for Specificity. “Selection” refers to a process in which many members of a display library are allowed to contact the target and those that bind are recovered and propagated. Here, selection was from a library having more than 10¹⁰ members. “Screening” refers to a process in which isolated members of the library are tested singly for binding to the target. Through automation, thousands of candidates may be screened in a highly parallel process. The display library selection methods described herein can include a selection process that discards display library members that bind to a non-target molecule. Examples of non-target molecules include, e.g., the Fc domain of the CD44Fc antigen. In one implementation, a so-called “negative selection” step is used to discriminate between the target and related non-target molecule and a related, but distinct non-target molecules. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target. The non-target and target binding data are compared (e.g., using a computer and software) to identify library members that specifically bind to CD44.

The display library selection and screening methods described herein can include a selection or screening process that selects for display library members that bind to specific sites on the target molecule. For example, elution with high concentration of HA selects for phage that bind to the HA-binding site of CD44. One can screen for a phage that binds to the HA-binding site of CD44 by performing ELISAs with and without HA in the buffer.

Diversity

Display libraries include variation at one or more positions in the displayed polypeptide. The variation at a given position can be synthetic or natural. For some libraries, both synthetic and natural diversity are included.

Synthetic Diversity. Libraries can include regions of diverse nucleic acid sequence that originate from artificially synthesized sequences. Typically, these are formed from degenerate oligonucleotide populations that include a distribution of nucleotides at each given position. The inclusion of a given sequence is random with respect to the distribution. One example of a degenerate source of synthetic diversity is an oligonucleotide that includes NNN wherein N is any of the four nucleotides in equal proportion.

Synthetic diversity can also be more constrained, e.g., to limit the number of codons in a nucleic acid sequence at a given trinucleotide to a distribution that is smaller than NNN. For example, such a distribution can be constructed using less than four nucleotides at some positions of the codon. In addition, trinucleotide addition technology can be used to further constrain the distribution.

So-called “trinucleotide addition technology” is described, e.g., in Wells et al. (1985) Gene 34:315-323, U.S. Pat. Nos. 4,760,025 and 5,869,644. Oligonucleotides are synthesized on a solid phase support, one codon (i.e., trinucleotide) at a time. The support includes many functional groups for synthesis such that many oligonucleotides are synthesized in parallel. The support is first exposed to a solution containing a mixture of the set of codons for the first position. The unit is protected so additional units are not added. The solution containing the first mixture is washed away and the solid support is deprotected so a second mixture containing a set of codons for a second position can be added to the attached first unit. The process is iterated to sequentially assemble multiple codons. Trinucleotide addition technology enables the synthesis of a nucleic acid that at a given position can encode a number of amino acids. The frequency of these amino acids can be regulated by the proportion of codons in the mixture. Further the choice of amino acids at the given position is not restricted to quadrants of the codon table as is the case if mixtures of single nucleotides are added during the synthesis. Synthetic oligonucleotides including randomized or spiked codons can be also be used for producing a library for an affinity maturation selection.

Natural Diversity. Libraries can include regions of diverse nucleic acid sequence that originate (or are synthesized based on) from different naturally-occurring sequences. An example of natural diversity that can be included in a display library is the sequence diversity present in immune cells (see also below). Nucleic acids are prepared from these immune cells and are manipulated into a format for polypeptide display. Another example of naturally diversity is the diversity of sequences among different species of organisms. For example, diverse nucleic acid sequences can be amplified from environmental samples, such as soil, and used to construct a display library. De Wildt J Mol Biol. Dec. 3, 1999;294(3):701-10 describe some exemplary characteristics of human immunoglobulin sequences.

Antibody Display Libraries

In one embodiment, the display library presents a diverse pool of polypeptides, each of which includes an immunoglobulin domain, e.g., an immunoglobulin variable domain. Display libraries are particular useful, for example for identifying human or “humanized” antibodies that recognize human antigens. Such antibodies can be used as therapeutics to treat human disorders such as cancer. Since the constant and framework regions of the antibody are human, these therapeutic antibodies may avoid themselves being recognized and targeted as antigens. The constant regions are also optimized to recruit effector functions of the human immune system. The in vitro display selection process surmounts the inability of a normal human immune system to generate antibodies against self-antigens.

A typical antibody display library displays a polypeptide that includes a VH domain and a VL domain. An “immunoglobulin domain” refers to a domain from the variable or constant domain of immunoglobulin molecules. Immunoglobulin domains typically contain two βsheets formed of about seven βstrands, and a conserved disulphide bond (see, e.g., A. F. Williams and A. N. Barclay 1988 Anni. Rev Immunol. 6:381-405). The display library can display the antibody as a Fab fragment (e.g., using two polypeptide chains) or a single chain Fv (e.g., using a single polypeptide chain). Other formats can also be used.

As in the case of the Fab and other formats, the displayed antibody can include a constant region as part of a light or heavy chain. In one embodiment, each chain includes one constant region, e.g., as in the case of a Fab. In other embodiments, additional constant regions are displayed.

Antibody libraries can be constructed by a number of processes (see, e.g., de Haard et al. (1999) J. Biol. Chem 274:18218-30; Hoogenboom et al. (1998) Immunotechnology 4:1-20. and Hoogenboom et al. (2000) Immunol Today 21:371-8. Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible.In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al. (2000) J. Mol. Biol. 296:57-86 describe a method for constructing CDR encoding oligonucleotides using trinucleotide synthesis and a template with engineered restriction sites for accepting the oligonucleotides.

In another process, an animal, e.g., a rodent, is immunized with the CD44. The animal is optionally boosted with the antigen to further stimulate the response. Then spleen cells are isolated from the animal, and nucleic acid encoding VH and/or VL domains is amplified and cloned for expression in the display library.

In yet another process, antibody libraries are constructed from nucleic acid amplified from naive germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

Nucleic acid encoding immunoglobulin domains can be obtained from the immune cells of, e.g., a human, a primate, mouse, rabbit, camel, or rodent. In one example, the cells are selected for a particular property. B cells at various stages of maturity can be selected. In another example, the B cells are naïve.

In one embodiment, fluorescent-activated cell sorting (FACS) is used to sort B cells that express surface-bound IgM, IgD, or IgG molecules. Further, B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin or pokeweed mitogen.

In still another embodiment, the cells are isolated from a subject that has an immunological disorder, e.g., systemic lupus erythematosus (SLE), rheumatoid arthritis, vasculitis, Sjogren syndrome, systemic sclerosis, or anti-phospholipid syndrome. The subject can be a human, or an animal, e.g., an animal model for the human disease, or an animal having an analogous disorder. In yet another embodiment, the cells are isolated from a transgenic non-human animal that includes a human immunoglobulin locus.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al. (2001) J Immunol. 166:2228). In another embodiment, the cells are naïve.

The nucleic acid encoding an immunoglobulin variable domain can be isolated from a natural repertoire by the following exemplary method. First, RNA is isolated from the immune cell. Full length (i.e., capped) mRNAs are separated (e.g. by dephosphorylating uncapped RNAs with calf intestinal phosphatase). The cap is then removed with tobacco acid pyrophosphatase and reverse transcription is used to produce the cDNAs.

The reverse transcription of the first (antisense) strand can be done in any manner with any suitable primer. See, e.g., de Haard et al. (1999) J. Biol. Chem 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. In another embodiment, poly-dT primers may be used (and may be preferred for the heavy-chain genes).

A synthetic sequence can be ligated to the 3′ end of the reverse transcribed strand. The synthetic sequence can be used as a primer binding site for binding of the forward primer during PCR amplification after reverse transcription. The use of the synthetic sequence can obviate the need to use a pool of different forward primers to fully capture the available diversity.

The variable domain-encoding gene is then amplified, e.g., using one or more rounds. If multiple rounds are used, nested primers can be used for increased fidelity. The amplified nucleic acid is then cloned into a display library vector.

Any method for amplifying nucleic acid sequences may be used for amplification. Methods that maximize and do not bias diversity are preferred. A variety of techniques can be used for nucleic acid amplification. The polymerase chain reaction (PCR; U.S. Pat. Nos. 4,683,195 and 4,683,202, Saiki, et al. (1985) Science 230, 1350-1354) utilizes cycles of varying temperature to drive rounds of nucleic acid synthesis. Transcription-based methods utilize RNA synthesis by RNA polymerases to amplify nucleic acid (U.S. Pat. Nos. 6,066,457; 6,132,997; 5,716,785; Sarkar et. al., Science (1989) 244: 331-34 ; Stofler et al., Science (1988) 239: 491). NASBA (U.S. Pat. Nos. 5,130,238; 5,409,818; and 5,554,517) utilizes cycles of transcription, reverse-transcription, and RNaseH-based degradation to amplify a DNA sample. Still other amplification methods include rolling circle amplification (RCA; U.S. Pat. Nos. 5,854,033 and 6,143,495) and strand displacement amplification (SDA; U.S. Pat. Nos. 5,455,166 and 5,624,825).

Secondary Screening Methods

After selecting candidate display library members that bind to a target, each candidate display library member can be further analyzed, e.g., to further characterize its binding properties for the target. Each candidate display library member can be subjected to one or more secondary screening assays. The assay can be for a binding property, a catalytic property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use the display library member directly, a recombinant polypeptide produced from the nucleic acid encoding a displayed polypeptide, or a synthetic peptide synthesized based on the sequence of a displayed polypeptide. Exemplary assays for binding properties include the following.

ELISA. Polypeptides encoded by a display library can also be screened for a binding property using an ELISA assay. For example, each polypeptide is contacted to a microtitre plate whose bottom surface has been coated with the target, e.g., a limiting amount of the target. The plate is washed with buffer to remove non-specifically bound polypeptides. Then the amount of the polypeptide bound to the plate is determined by probing the plate with an antibody that can recognize the polypeptide, e.g., a tag or constant portion of the polypeptide. The antibody is linked to an enzyme such as alkaline phosphatase, which produces a calorimetric product when appropriate substrates are provided. The polypeptide can be purified from cells or assayed in a display library format, e.g., as a fusion to a filamentous bacteriophage coat. Alternatively, cells (e.g., live or fixed) that express the target molecule, e.g., activated CD44, can be plated in a microtitre plate and used to test the affinity of the peptides/antibodies present in the display library or obtained by selection from the display library.

In another version of the ELISA assay, each polypeptide of a diversity strand library is used to coat a different well of a microtitre plate. The ELISA then proceeds using a constant target molecule to query each well.

Homogeneous Binding Assays. The binding interaction of candidate polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is Alpha Screen (Packard Bioscience, Meriden Conn.). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate polypeptide is attached to the display library vehicle, e.g., a bacteriophage.

Surface Plasmon Resonance (SPR). The binding interaction of a molecule isolated from a display library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by crystallography or NMR). As a result, an understanding of the physical interaction between the protein and its target can be formulated and used to guide other design processes.

Protein Arrays. Polypeptides identified from the display library can be immobilized on a solid support, for example, on a bead or an array. For a protein array, each of the polypeptides is immobilized at a unique address on a support. Typically, the address is a two-dimensional address. Protein arrays are described below (see, e.g., Diagnostics).

Functional Assays

Cellular Assays. Candidate polypeptides can be selected from a library by transforming the library into a host cell; the library could have been previously identified from a display library. For example, the library can include vector nucleic acid sequences that include segments that encode the polypeptides and that direct expression, e.g., such that the polypeptides are produced within the cell, secreted from the cell, or attached to the cell surface. The cells can be screened or selected for polypeptides that bind to the CD44, e.g., as detected by a change in a cellular phenotype or a cell-mediated activity. For example, in the case of an antibody that binds to the CD44, the activity may be cell or complement-mediated cytotoxicity.

In another embodiment, the library of cells is in the form of a cellular array. The cellular array can likewise be screened for any appropriate detectable activity.

In other embodiments, cellular assays can be used to test the effects of proteins/antibodies obtained from the display library. Such assays can be used, e.g., to measure cellular adhesion or migration, or lymphocyte rolling. Techniques for measuring cellular adhesion, migration, and lymphocyte rolling are well known in the art. See, e.g., Siegelman et al. (1999), J Leukoc Biol 66(2):315-21; Hidalgo et al. (2002), J Hematother Stem Cell Res 11(3):539-47; Bourguignon et al. (2002), J Biol Chem July 26 (published online); and Zhang et al. (2002), Cancer Res 62(14):3962-5.

An exemplary in vitro cell proliferation assay is the Cell Titer 96® Aqueous non-radioactive cell proliferation assay (Promega, Wis.). In an exemplary implementation, cells are seeded into a well of a microtitre plate. The test compound, e.g., a CD44-binding antibody described herein, is added to the well. The cells are incubated for 4 days at 37° C. in 5% CO₂ atmosphere. After the incubation MTS/PMS MTS/PMS (3-(4,5-dimethylthiazol-2-yl)-5-(3-carboxymethoxyphenyl)-2-(4-sulfophenyl)-2H-tetrazolium/phenazine formazan) is added according to the kit instructions. Absorbance is monitored at 490 nm. An IC₅₀ value for the cells is calculated based on the fraction of surviving cells. Cells that can be used include CD44 expressing cells, e.g., A431 cells (ATCC # CRL 1555; epidermoid carcinoma of the vulva) and FaDu cells (ATCC # HTB43; squamous cell carcinoma of the pharynx). Controls for comparison can include wells that lack the test compound or wells that include a cell that does not express CD44, e.g., A459 cells.

Exemplary in vivo assays to evaluate the anti-tumor effect of a CD44-binding antibody includes nude mouse tumor models. In an exemplary implementation, a nude mouse is xenografted with a human tumor that includes CD44-expressing cells. For example, 106 tumor cells can be transplanted subcutaneously into the flank of a nude mouse, e.g., the NMRI-nu/nu mouse. Exemplary human tumor cells include carcinoma cells, e.g., A431 (ATCC # CRL 1555) and breast carcinoma cells, e.g., MDA-MB453 (ATCC# HTB-131). When the tumor reaches an average size of between 100-180 mm³, the test compound or control composition is administered to the mouse, e.g., by intravenous injection. It is possible to use this assay to monitor the effect of different dosages, e.g., about 0.1, 0.5, 1.0, 5.0, 10, 20 or 25 mg/kg/day or ranges therebetween. Animals are monitored by evaluating tumor size. For example, a tumor response can be rated as complete if the tumor entirely disappears or as a partial response if the tumor volume decreases after treatment, but then begins regrowing. The animals can also be monitored to evaluate tolerability of the treatment, e.g., by monitoring animal weight.

Ligand Production

Standard recombinant nucleic acid methods can be used to construct nucleic acids that encode a protein ligand that binds to CD44 and to express the protein ligand.

Methods well known to those skilled in the art can be used to construct vectors containing a polynucleotide encoding a ligand and appropriate transcriptional/translational control signals. These methods can include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989).

Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be shuffled into a bacterial cell that cannot suppress a stop codon. In this case, the Fab is not fused to the gene III protein and is secreted into the media.

Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibodies are produced in mammalian cells. Preferred mammalian host cells for expressing the clone antibodies or antigen-binding fragments thereof include Chinese Hamster Ovary (CHO cells) (including dhfr- CHO cells, described in Urlaub and Chasin (1980) Proc. Natl. Acad. Sci. USA 77:4216-4220, used with a DFFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequence encoding the protein ligand, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, a recombinant expression vector encoding both the antibody heavy chain and the antibody light chain is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G.

For antibodies that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

It is also possible to produce antibodies that bind to CD44 by immunization, e.g., using an animal, e.g., with natural, human, or partially human immunoglobulin loci. Non-human antibodies can also be modified to include substitutions for human immunoglobulin sequences, e.g., consensus human amino acid residues at particular positions, e.g., at one or more of the following positions (preferably at least five, ten, twelve, or all): (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

CD44 production. Method for producing CD44 ectodomain protein, CD44 protein, and CD44-containing liposomes are known in the art. See, e.g., U.S. Pat. No. 6,432,405.

Pharmaceutical Compositions

A CD44-binding protein ligand can be a component of a composition, e.g., a pharmaceutically acceptable composition. To prepare one such composition, the ligand is formulated together with a pharmaceutically acceptable carrier. As used herein, “pharmaceutical compositions” encompass labeled ligands for in vivo imaging as well as therapeutic compositions.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., protein ligand may be coated in a material to protect the compound from the action of acids and other natural conditions that may inactivate the compound.

“pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the CD44-binding ligand is administered by intravenous infusion or injection. In another preferred embodiment, the CD44-binding ligand is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot # 7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The CD44-binding protein ligands described herein can be administered by a variety of methods known in the art, although for many applications, the preferred route/mode of administration is intravenous injection or infusion. For example, for therapeutic applications, the CD44-binding ligand can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the ligand may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with a material that prevents its inactivation.

Pharmaceutical compositions can be administered with medical devices known in the art. For example, a pharmaceutical composition can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of implants and modules include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, a CD44-binding protein ligand can be formulated to ensure proper distribution in vivo. For example, the blood-brain barrier (BBB) excludes many highly hydrophilic compounds. To ensure that a therapeutic compound crosses the BBB (if desired), it can be formulated, for example, in liposomes. For methods of manufacturing liposomes, see, e.g., U.S. Pat. Nos. 4,522,811; 5,374,548; and 5,399,331. The liposomes may include one or more moieties which are selectively transported into specific cells or organs, thus enhance targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a CD44-binding antibody is 0.1-20 mg/kg, more preferably 1-10 mg/kg. The CD44-binding antibody can be administered by intravenous infusion at a rate of less than 30, 20, 10, 5, or 1 mg/min to reach a dose of about 1 to 100 mg/m² or about 5 to 30 mg/m². For ligands smaller in molecular weight than an antibody, appropriate amounts can be proportionally less. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

A pharmaceutical composition may include a “therapeutically effective amount” or a “prophylactically effective amount” of a CD44-binding antibody. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein ligand to elicit a desired response in the individual. A therapeutically effective amount is also one in which any toxic or detrimental effects of the composition are outweighed by the therapeutically beneficial effects. A “therapeutically effective dosage” preferably inhibits a measurable parameter, e.g., inflammation or tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Kits can be prepared that include a CD44-binding antibody and instructions for use, e.g., treatment, prophylactic, or diagnostic use. In one embodiment, the instructions for diagnostic applications include the use of the CD44-binding antibody (e.g., antibody or antigen-binding fragment thereof, or other polypeptide or peptide) to detect CD44, in vitro, e.g., in a sample, e.g., a biopsy or cells from a patient having an inflammatory disorder or a cancer or neoplastic disorder, or in vivo. In another embodiment, the instructions for therapeutic applications include suggested dosages and/or modes of administration in a patient with a cancer or neoplastic disorder. The kit can further contain a least one additional reagent, such as a diagnostic or therapeutic agent, e.g., a diagnostic or therapeutic agent as described herein, and/or one or more additional CD44-binding ligands, formulated as appropriate, in one or more separate pharmaceutical preparations.

Stabilization and Retention

In one embodiment, a CD44 ligand (e.g., a CD44 binding antibody described herein) is physically associated with a moiety that improves its stabilization and/or retention in circulation, e.g., in blood, serum, lymph, or other tissues.

For example, a CD44 ligand can be associated with a polymer, e.g., a substantially non-antigenic polymers, such as polyalkylene oxides or polyethylene oxides. Suitable polymers will vary substantially by weight. The polymers can have average molecular weights in the ranges of from about 200 to about 35,000 Daltons, from about 1,000 to about 15,000 and 2,000 to about 12,500 Daltons.

For example, a CD44 ligand can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

Other compounds can also be attached to the same polymer, e.g., a cytotoxin, a label, or another targeting agent, e.g., another CD44 ligand or an unrelated ligand. Mono-activated, alkoxy-terminated polyalkylene oxides (PAO's), e.g., monomethoxy-terminated polyethylene glycols (mPEG's); C₁₋₄ alkyl-terminated polymers; and bis-activated polyethylene oxides (glycols) can be used for crosslinking. See, e.g., U.S. Pat. No. 5,951,974.

In one embodiment, the polymer prior to cross-linking need not be, but preferably is, water soluble. Generally, after crosslinking, the product is water soluble, e.g., exhibits a water solubility of at least about 0.01 mg/ml, and more preferably at least about 0.1 mg/ml, and still more preferably at least about 1 mg/ml. In addition, the polymer should not be highly immunogenic in the conjugate form, nor should it possess viscosity that is incompatible with intravenous infusion or injection if the conjugate is intended to be administered by such routes.

In one embodiment, the polymer contains only a single group which is reactive. This helps to avoid cross-linking of protein molecules. However, it is within the scope herein to maximize reaction conditions to reduce cross-linking, or to purify the reaction products through gel filtration or ion exchange chromatography to recover substantially homogenous derivatives. In other embodiments, the polymer contains two or more reactive groups for the purpose of linking multiple ligands to the polymer backbone. Again, gel filtration or ion exchange chromatography can be used to recover the desired derivative in substantially homogeneous form.

The molecular weight of the polymer can range up to about 500,000 D, and preferably is at least about 20,000 D, or at least about 30,000 D, or at least about 40,000 D. The molecular weight chosen can depend upon the effective size of the conjugate to be achieved, the nature (e.g. structure, such as linear or branched) of the polymer, and the degree of derivatization.

The covalent crosslink can be used to attach a CD44 ligand to a polymer, for example, by crosslinking the polymer to the N-terminal amino group and epsilon amino groups found on lysine residues, as well as other amino, imino, carboxyl, sulfhydryl, hydroxyl or other hydrophilic groups. The polymer may be covalently bonded directly to the CD44 ligand without the use of a multifunctional (ordinarily bifunctional) crosslinking agent. Covalent binding to amino groups is accomplished by known chemistries based upon cyanuric chloride, carbonyl diimidazole, aldehyde reactive groups (PEG alkoxide plus diethyl acetal of bromoacetaldehyde; PEG plus DMSO and acetic anhydride, or PEG chloride plus the phenoxide of 4-hydroxybenzaldehyde, activated succinimidyl esters, activated dithiocarbonate PEG, 2,4,5-trichlorophenylcloroformate or P-nitrophenylcloroformate activated PEG.) Carboxyl groups can be derivatized by coupling PEG-amine using carbodiimide. Sulfhydryl groups can be derivatized by coupling to maleimido-substituted PEG (e.g. alkoxy-PEG amine plus sulfosuccinimidyl 4-(N-maleimidomethyl)cyclohexane-1-carboxylate) WO 97/10847 or PEG-maleimide commercially available from Shearwater Polymers, Inc., Huntsville, Ala.). Alternatively, free amino groups on the ligand (e.g. epsilon amino groups on lysine residues) can be thiolated with 2-imino-thiolane (Traut's reagent) and then coupled to maleimide-containing derivatives of PEG, e.g., as described in Pedley et al., Br. J. Cancer, 70: 1126-1130 (1994).

Functionalized PEG polymers that can be attached to a CD44 ligand are available, e.g., from Shearwater Polymers, Inc. (Huntsville, Ala.). Such commercially available PEG derivatives include, e.g., amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives may vary depending on the CD44 ligand, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer.

The conjugates of a CD44-binding ligand and a polymer can be separated from the unreacted starting materials, e.g., by gel filtration or ion exchange chromatography, e.g., HPLC. Heterologous species of the conjugates are purified from one another in the same fashion. Resolution of different species (e.g. containing one or two PEG residues) is also possible due to the difference in the ionic properties of the unreacted amino acids. See, e.g., WO 96/34015.

A conjugate of a CD44-binding ligand and a polymer can be administered to the subject in an amount effective to maintain a desired concentration in a subject for at least 4, 8, 12, 24, or 72 hours. Because the conjugate is stabilized and may have an extended circulatory half-life, it may be possible to administer the conjugate as part of a regimen no more than once every 12, 24, 48, or 72 hours or no more than once every three, four, five, six, ten, twelve, or fourteen days. The conjugate may have a beta phase half life of at least 4, 6, 8, 8.5, 9, 10, 12, 20, 24, 30, 42, 48, or 50 hours. The beta phase may be at least 40, 50, 60, 70, or 80% of the amplitude.

Treatments

Protein ligands that bind to CD44 (e.g., those described herein) have therapeutic and prophylactic utilities. For example, these ligands can be administered to cells in culture, e.g. in vitro or ex vivo, or in a subject, e.g., in vivo, to treat, prevent, and/or diagnose a variety of disorders, such as inflammatory diseases and cancers.

As used herein, the term “treat” or “treatment” is defined as the application or administration of a CD44-binding antibody, alone or in combination with, a second agent to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder. Treating a cell refers to the inhibition, ablation, killing of a cell in vitro or in vivo, or otherwise reducing capacity of a cell, e.g., an aberrant cell, to mediate a disorder, e.g., a disorder as described herein (e.g., a cancerous disorder). In one embodiment, “treating a cell” refers to a reduction in the activity and/or proliferation of a cell, e.g., a hyperproliferative cell. Such reduction does not necessarily indicate a total elimination of the cell, but a reduction, e.g., a statistically significant reduction, in the activity or the number of the cell.

As used herein, an amount of a CD44-binding ligand effective to treat a disorder, or a “therapeutically effective amount” refers to an amount of the ligand which is effective, upon single or multiple dose administration to a subject, in treating a cell, e.g., white blood cell (e.g., a B cell, T cell, macrophage, or neutrophil), parenchymal cell, or cancer cell (e.g., a CD44-expressing cancer cell, particularly a metastatic cell thereof), or in prolonging curing, alleviating, relieving or improving a subject with a disorder as described herein beyond that expected in the absence of such treatment. As used herein, “inhibiting the growth” of the neoplasm refers to slowing, interrupting, arresting or stopping its growth and metastases and does not necessarily indicate a total elimination of the neoplastic growth.

As used herein, an amount of a CD44-binding ligand effective to prevent a disorder, or a “a prophylactically effective amount” of the ligand refers to an amount of a CD44-binding ligand, e.g., a CD44-binding antibody described herein, which is effective, upon single- or multiple-dose administration to the subject, in preventing or delaying the occurrence of the onset or recurrence of a disorder, e.g., an inflammatory disorder or a cancer.

The terms “induce”, “inhibit”, “potentiate”, “elevate”, “increase”, “decrease” or the like, e.g., which denote quantitative differences between two states, refer to a difference, e.g., a statistically significant difference, between the two states. For example, “an amount effective to inhibit the proliferation of the CD44-expressing hyperproliferative cells” means that the rate of growth of the cells will be different, e.g., statistically significantly different, from the untreated cells.

As used herein, the term “subject” is intended to include human and non-human animals. Preferred human animals include a human patient having a disorder characterized by abnormal cell proliferation or cell differentiation. The term “non-human animals” includes all non-human vertebrates, e.g., non-mammals (such as chickens, amphibians, reptiles) and non-human mammals, such as non-human primates, sheep, dog, cow, pig, etc.

In one embodiment, the subject is a human subject. Alternatively, the subject can be a mammal expressing a CD44-like antigen with which an antibody cross-reacts. A CD44-binding antibody can be administered to a human subject for therapeutic purposes (see, e.g., below). Moreover, a CD44-binding ligand can be administered to a non-human mammal expressing the CD44-like antigen to which the ligand binds (e.g., a primate, pig or mouse) for veterinary purposes or as an animal model of human disease. Regarding the latter, such animal models may be useful for evaluating the therapeutic efficacy of the ligand (e.g., testing of dosages and time courses of administration).

In one embodiment, the CD-44 binding antibody is used to treat (e.g., ablating or killing) a cell (e.g., a non-cancerous cell, e.g., a normal, benign or hyperplastic cell, or a cancerous cell, e.g., a malignant cell, e.g., cell found in a solid tumor, a soft tissue tumor, or a metastatic lesion (e.g., a cell found in renal, urothelial, colonic, rectal, pulmonary, breast or hepatic, cancers and/or metastasis)). The methods can include the steps of contacting the cell with a CD44-binding ligand, e.g., a CD44-binding antibody described herein, in an amount sufficient to treat, e.g., ablate or kill, the cell.

The subject method can be used on cells in culture, e.g. in vitro or ex vivo. For example, cancerous or metastatic cells (e.g., renal, urothelial, colon, rectal, lung, breast, ovarian, prostatic, or liver cancerous or metastatic cells) can be cultured in vitro in culture medium and the contacting step can be effected by adding the CD44-binding ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the CD44-binding ligand to the subject under conditions effective to permit both binding of the ligand to the cell and the treating, e.g., the killing or ablating of the cell.

The method can be used to treat a cancer. As used herein, the terms “cancer”, “hyperproliferative”, “malignant”, and “neoplastic” are used interchangeably, and refer to those cells an abnormal state or condition characterized by rapid proliferation or neoplasm. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using the CD44-binding antibodies described herein. The CD44-binding antibodies that antagonize CD44 activity can be particularly useful for treating cancers that include CD44-expressing cells.

The subject method can be useful in treating malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the CD44-binding protein can be used to treat various myeloid disorders including, but not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

Methods of administering CD44-binding ligands are also described in “Pharmaceutical Compositions”. Suitable dosages of the molecules used will depend on the age and weight of the subject and the particular drug used. The ligands can be used as competitive agents to inhibit, reduce an undesirable interaction, e.g., between a natural or pathological agent and the CD44.

In one embodiment, the CD44-binding ligands are used to kill or ablate cancerous cells and normal, benign hyperplastic, and cancerous cells in vivo. The ligands can be used by themselves or conjugated to an agent, e.g., a cytotoxic drug or radioisotope. This method includes: administering the ligand alone or attached to a cytotoxic drug, to a subject requiring such treatment.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell. In cancer therapeutic embodiment, the term “cytotoxic agent” is used interchangeably with the terms “anti-cancer” or “anti-tumor” to mean an agent, which inhibits the development or progression of a neoplasm, particularly a solid tumor, a soft tissue tumor, or a metastatic lesion.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: antitubulin/antimicrotubule, e.g., paclitaxel, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5 - fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trifluoromethyl) propionanilide.

In one embodiment, the CD44-binding antibody is conjugated to a maytansinoid, e.g., a derivative of maytansine (CAS 35846538) or a C-3 ester of maytansinol. For example, the maytansinoid can be linked by a covalent bond such as a disulfide moiety. It is also possible to link multiple maytansinoid moieties to a single antibody molecule, e.g., at least 2, 3, or 4 maytansinoid moieties. Maytansinoids suitable for conjugating to antibodies for use in cancer therapy and methods for preparing such are known; see, e.g., Chari R V J, et al. Cancer Research 52:127-31, 1992; Liu C. et al., Proc Natl Acad Sci USA 93:8618-23, 1996; U.S. Pat. No. 5,208,020. In one embodiment, the CD44-binding antibody is conjugated to N 2-deacetyl- N^{2′}-(3-mercapto-1-oxopropyl)-Maytansine (CAS Number 139504-50-0, e.g., DM1). The maytansinoid can be a maytansinol derivative linked to the antibody molecule via a disulfide bridge at the C-3 position of maytansinol.

Since the CD44-binding ligands recognize CD44-expressing cancer cells, e.g., cancerous lung, liver, colon, breast, ovarian, epidermal, laryngeal, and cartilage cells, and particularly metastatic cells thereof, any such cells to which the ligands bind are destroyed. Alternatively, the ligands bind to cells in the vicinity of the cancerous cells and kill them, thus indirectly attacking the cancerous cells which may rely on surrounding cells for nutrients, growth signals and so forth. Thus, the CD44-binding ligands (e.g., modified with a cytotoxin) can selectively kill or ablate cells in cancerous tissue (including the cancerous cells themselves).

The ligands may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy α-emitters, as described herein.

Enzymatically active toxins and fragments thereof are exemplified by diphtheria toxin A fragment, nonbinding active fragments of diphtheria toxin, exotoxin A (from Pseudomnonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. Procedures for preparing enzymatically active polypeptides of the immunotoxins are described in WO84/03508 and WO85/03508, and in the appended Examples below. Examples of cytotoxic moieties that can be conjugated to the antibodies include adriamycin, chlorambucil, daunomycin, methotrexate, neocarzinostatin, and platinum.

In the case of polypeptide toxins, recombinant nucleic acid techniques can be used to construct a nucleic acid that encodes the ligand (or a polypeptide component thereof) and the cytotoxin (or a polypeptide component thereof) as translational fusions. The recombinant nucleic acid is then expressed, e.g., in cells and the encoded fusion polypeptide isolated.

Procedures for conjugating protein ligands (e.g., antibodies) with the cytotoxic agents have been previously described. Procedures for conjugating chlorambucil with antibodies are described by Flechner (1973) European Journal of Cancer, 9:741-745; Ghose et al. (1972) British Medical Journal, 3:495-499; and Szekerke, et al. (1972) Neoplasma, 19:211-215. Procedures for conjugating daunomycin and adriamycin to antibodies are described by Hurwitz, E. et al. (1975) Cancer Research, 35:1175-1181 and Arnon et al. (1982) Cancer Surveys, 1:429-449. Procedures for preparing antibody-ricin conjugates are described in U.S. Pat. No. 4,414,148 and by Osawa, T., et al. (1982) Cancer Surveys, 1:373-388 and the references cited therein. Coupling procedures as also described in EP 86309516.2. For example, antibody molecules can be modified with crosslinking reagents such as N-succinimidyl 3-(2-pyridldithio)propionate (SPDP), 4-succinimidyl-oxycarbonyl-.alpha.-methyl-.alpha.-(2-pyridyldithio)-tolue-ne (SMPT), N-succinimidyl-3-(2-pyridyldithio)-butyrate (SDPB), N-succinimidyl-4-(2-pyridyldithio)pentanoate (SPP), N-succinimidyl-5-(2-pyridyldithio)pentanoate, 2-iminothiolane, or acetylsuccinic anhydride by known methods. See, Carlsson et al, Biochem. J. 173:723-737, 1978; Blattler, et al. Biochem. 24:1516-1524, 1985; Lambert et al. Biochem. 22:3913-3920, 1983; Klotz et al, Arch. Biochem. Biophys. 96:605, 1962; Liu et al, Biochem. 18:690, 1979; Blakey and Thorpe, Immunoconjugates and Radiopharmaceuticals 1:1-16, 1988; Worrell et al. Anti-Cancer Drug Design 1: 179-184, 1986. In one embodiment, the linker moiety is a 4-thiopentanoate derived from SPP.

To kill or ablate normal, benign hyperplastic, or cancerous cells, a first protein ligand is conjugated with a prodrug which is activated only when in close proximity with a prodrug activator. The prodrug activator is conjugated with a second protein ligand, preferably one which binds to a non-competing site on the target molecule. Whether two protein ligands bind to competing or non-competing binding sites can be determined by conventional competitive binding assays. Blakely et al., (1996) Cancer Research, 56:3287-3292 described some suitable drug-prodrug pairs.

Alternatively, the CD44-binding ligand can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985). Other suitable radioisotopes include α-emitters, such as ²¹²Bi, ²¹³Bi, and ²¹¹At, and β-emitters, such as ¹⁸⁶Re and ⁹⁰Y. Moreover, Lu¹¹⁷ may also be used as both an imaging and cytotoxic agent.

Radioimmunotherapy (RIT) using antibodies labeled with ¹³¹I, ⁹⁰Y, and ¹⁷⁷Lu is under intense clinical investigation. There are significant differences in the physical characteristics of these three nuclides and as a result, the choice of radionuclide is very critical in order to deliver maximum radiation dose to the tumor. The higher beta energy particles of 90Y may be good for bulky tumors. The relatively low energy beta particles of 131I are ideal, but in vivo dehalogenation of radioiodinated molecules is a major disadvantage for internalizing antibody. In contrast, ¹⁷⁷Lu has low energy beta particle with only 0.2-0.3 mm range and delivers much lower radiation dose to bone marrow compared to 90Y. In addition, due to longer physical half-life (compared to 90Y), the tumor residence times are higher. As a result, higher activities (more mCi amounts) of ¹⁷⁷Lu labeled agents can be administered with comparatively less radiation dose to marrow. There have been several clinical studies investigating the use of ¹⁷⁷Lu labeled antibodies in the treatment of various cancers. (Mulligan T et al. (1995) Clin Cancer Res. 1: 1447-1454; Meredith R F, et al. (1996) J Nucl Med 37:1491-1496; Alvarez R D, et al. (1997) Gynecologic Oncology 65: 94-101).

The CD44-binding ligands can be used directly in vivo to eliminate antigen- expressing cells via natural complement-dependent cytotoxicity (CDC) or antibody-dependent cellular cytotoxicity (ADCC). A CD44-binding protein described herein can include complement binding effector domain, such as the Fc portions from IgG1, −2, or −3 or corresponding portions of IgM which bind complement. In one embodiment, a population of target cells is ex vivo treated with a CD44-binding antibody and appropriate effector cells. The treatment can be supplemented by the addition of complement or serum containing complement. Further, phagocytosis of target cells that express CD44 and are coated with a CD44-binding antibody can be improved by binding of complement proteins. In another embodiment target, cells coated with the protein ligand which includes a complement binding effector domain are lysed by complement.

A CD44-binding antibody can also be used for prophylaxis. For example, the antibody can be used to prevent or delay development or progression of cancers, e.g.,., by inhibiting or killing a CD-44 expressing cell.

Use of the therapeutic methods described herein to treat cancers has a number of benefits. Since the protein ligands specifically recognize CD44, other tissue is spared and high levels of the agent are delivered directly to the site where therapy is required. Treatments can be effectively monitored with clinical parameters. Alternatively, these parameters can be used to indicate when such treatment should be employed.

CD44-binding proteins can be administered in combination with one or more of the existing modalities for treating cancers, including, but not limited to: surgery; radiation therapy, and chemotherapy.

CD44-binding proteins can be administered in combination with one or more of the existing modalities for treating an inflammatory disease or disorder. Exemplary inflammatory diseases or disorders include: acute and chronic immune and autoimmune pathologies, such as, but not limited to, rheumatoid arthritis (RA), juvenile chronic arthritis (JCA), psoriasis, graft versus host disease (GVHD), scleroderma, diabetes mellitus, allergy; asthma, acute or chronic immune disease associated with an allogenic transplantation, such as, but not limited to, renal transplantation, cardiac transplantation, bone marrow transplantation, liver transplantation, pancreatic transplantation, small intestine transplantation, lung transplantation and skin transplantation; chronic inflammatory pathologies such as, but not limited to, sarcoidosis, chronic inflammatory bowel disease, ulcerative colitis, and Crohn's pathology or disease; vascular inflammatory pathologies, such as, but not limited to, disseminated intravascular coagulation, atherosclerosis, Kawasaki's pathology and vasculitis syndromes, such as, but not limited to, polyarteritis nodosa, Wegener's granulomatosis, Henoch-Schonlein purpura, giant cell arthritis and microscopic vasculitis of the kidneys; chronic active hepatitis; Sjogren's syndrome; psoriatic arthritis; enteropathic arthritis; reactive arthritis and arthritis associated with inflammatory bowel disease; and uveitis. A CD44-binding protein can be used to treat or prevent one of the foregoing diseases or disorders. For example, the protein can be administered (locally or systemically) in an mount effective to ameliorate at least one symptom of the respective disease or disorder. The protein may also ameliorate inflammation, e.g., an indicator of inflammation, e.g., such as local temperature, swelling (e.g., as measured), redness, local or systemic white blood cell count, presence or absence of neutrophils, elastase activity, and so forth.

Cell Grafting and Transplantation

In one aspect, a CD44 binding antibody that has agonist activity is used to treat a subject (e.g., a human patient) prior to introducing exogenous cells into the subject. The antibody may be used, e.g., to decrease the rate of rejection of the exogenous cells, e.g., graft rejection. For example, the antibody can be used for hematopoietic stem cell transplantation (HSCT), e.g., a non-myeloablative HSCT regimen. The antibody can be administered prior to the transplantation, e.g., to sensitize cells prior to irradiating the subject. For example, the subject may have a hematopoietic cancer, e.g., a leukemia, such as chronic myelocytic leukemia (CML), chronic lymphocytic leukemia (CLL), acute myelocytic leukemia (AML), multiple myeloma (MM), or a non-Hodgkin lymphoma (NHL). The human subject may be elderly (e.g., older than 60, 65, 70, 75, 80, 85, or 90 years of age) or may have additional co-morbidities.

Prior to transplantation, the subject is irradiated, e.g., with a low dose of total body irradiation (TBI), for example, to less than 500, 400, 300, 250, 200, 180, 160, 140, 100, or 50 cGy or less. In one implementation, TBI can be given at between 5-10 cGy/minute, e.g., from a dual cobalt 60 source or a linear accelerator. In other implementations, TBI may not be necessary. For example, alternate means can be used to achieve cell killing, e.g., NK cell ablation.

The cells used for transplantation can be allogenic (from the same species as the subject) or xenogenic (from another species). In one embodiment, the cells are matched for one or more antigens, e.g., MHC antigens. In another embodiment, unmatched cells are used.

After transplantation, the subject can be further treated with an immunosuppressant. For example, the subject can be treated with mycophenolate mofetil (MMF) and/or cyclosporine.

The outcome of transplantation can be evaluated by determining the degree of chimerism after transplantation. For example, fluorescence in situ hybridization to detect X or Y chromosomes for sex-mismatched transplants or PCR to detect other genetic polymorphisms can be used. Between 1% and 95% PB donor T cells can be considered to be mixed chimerism.

In one embodiment, the HSCT regimen includes infusing peripheral blood stem cells (PBSCs) within zero, one or two days of TBI. For example, granulocyte colony stimulating factor (G-CSF) mobilized PBSCs from a donor can be used. After chimerism is detected, e.g., after about 20, 50, 80, 100, 150, 200, 250, or 300 days later, donor leukocytes can be infused, e.g., about 10⁶, 10^{7,} or 10⁸ CD³⁺ cells/kg, or ranges therebetween.

In one embodiment, the subject receives fludarabine in addition to the CD44-binding antibody prior to TBI. In other embodiments, other treatments can also be used in combination with sensitization with a CD44-binding antibody. Examples of such other treatments include: anti-lymphocyte globulin (ALG), anti-CD8 antibodies, or an alkylating agent such as cyclophosphamide.

In one aspect, a CD44-binding antibody that has agonist activity is used to modulate (e.g., control) graft-versus-host disease (GVHD), and host-versus-graft reactions (HVG) (e.g., a residual HVG after immunosuppression). The antibody can be used in a method that includes administering the antibody one or more times, before, during, or after transplantation of an exogenous cell, e.g., donor hematopoietic cells, e.g., leukocytes.

Other exemplary cells that can be introduced into a recipient subject include: xenogeneic, allogeneic, genetically engineered syngeneic, or genetically engineered autologous stem cells. For example, the stem cells can include an exogenous nucleic acid that expresses a cell surface protein, e.g., an MHC protein, e.g., an MHC Class I or Class II protein, e.g.,. matched or unmatched to the recipient. Exemplary sources of xenogeneic cells include porcine cells, primate cells, and human cells. It is also possible to use cells that are removed from the recipient, i.e., the recipient's own cells, wherein the removed cells are subsequently modified, e.g., by introduction of an exogenous nucleic acid, e.g., that expresses a cell surface protein.

Retroviral transformation allows production of transgenic bone marrow cells, preferably autologous bone marrow cells, expressing allogeneic or xenogeneic MHC genes. Expression of the transgenic MHC genes confers tolerance to grafts which exhibit the products of these or closely related MHC genes. Thus, these methods provide for the induction of specific transplantation tolerance by somatic transfer of MHC genes. See, e.g., U.S. Ser. No. 2002-0,168,348.

Without intending to be bound by theory, an S5-like CD44-binding antibody may improve success of engraftment after introduction of exogenous cells by one or more mechanisms. The antibody may sensitize host NK cells and prime them for ablation, e.g., by low dose irradiation. The antibody may enhance hematopoiesis of donor cells.

The methods described herein can be used to induce tolerance for any introduction of exogenous cells. Examples of cell engraftments include: solid organ transplantation (e.g., liver, kidney, lung, or heart), hematologic disorders, including aplastic anemia, severe combined immunodeficiency (SCID) states, thalassemia, diabetes and other autoimmune disease states, sickle cell anemia, and some enzyme deficiency states. A specific example of cell engraftment that can be achieved is partial engraftment of allogeneic or even xenogeneic bone marrow that creates a mixed host/donor chimeric state with preservation of immunocompetence and resistance to GVHD.

Diagnostic Uses

Protein ligands that bind to CD44 (e.g., those described herein) have in vitro and in vivo diagnostic, therapeutic and prophylactic utilities.

In one aspect, the invention provides a diagnostic method for detecting the presence of a CD44, in vitro (e.g., a biological sample, such as tissue, biopsy, e.g., a cancerous tissue) or in vivo (e.g., in vivo imaging in a subject).

The method includes: (i) contacting a sample with CD44-binding ligand; and (ii) detecting formation of a complex between the CD44-binding ligand and the sample. The method can also include contacting a reference sample (e.g., a control sample) with the ligand, and determining the extent of formation of the complex between the ligand and the sample relative to the same for the reference sample. A change, e.g., a statistically significant change, in the formation of the complex in the sample or subject relative to the control sample or subject can be indicative of the presence of CD44 in the sample.

Another method includes: (i) administering the CD44-binding ligand to a subject; and (iii) detecting formation of a complex between the CD44-binding ligand, and the subject. The detecting can include determining location or time of formation of the complex.

The CD44-binding ligand can be directly or indirectly labeled with a detectable substance to facilitate detection of the bound or unbound antibody. Suitable detectable substances include various enzymes, prosthetic groups, fluorescent materials, luminescent materials and radioactive materials.

Complex formation between the CD44-binding ligand and CD44 can be detected by measuring or visualizing either the ligand bound to the CD44 or unbound ligand. Conventional detection assays can be used, e.g., an enzyme-linked immunosorbent assays (ELISA), a radioimmunoassay (RIA) or tissue immunohistochemistry. Further to labeling the CD44-binding ligand, the presence of CD44 can be assayed in a sample by a competition immunoassay utilizing standards labeled with a detectable substance and an unlabeled CD44-binding ligand. In one example of this assay, the biological sample, the labeled standards and the CD44 binding agent are combined and the amount of labeled standard bound to the unlabeled ligand is determined. The amount of CD44 in the sample is inversely proportional to the amount of labeled standard bound to the CD44 binding agent.

Fluorophore and chromophore labeled protein ligands can be prepared. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer (1968) Science, 162:526 and Brand, L. et al. (1972) Annual Review of Biochemistry, 41:843-868. The protein ligands can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the protein ligand can be used to detect the presence or localization of the CD44 in a sample, e.g., using fluorescent microscopy (such as confocal or deconvolution microscopy).

Histological Analysis. Immunohistochemistry can be performed using the protein ligands described herein. For example, in the case of an antibody, the antibody can synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation for binding, the preparation is washed to remove unbound antibody. The preparation is then analyzed, e.g., using microscopy, to identify if the antibody bound to the preparation.

Of course, the antibody (or other polypeptide or peptide) can be unlabeled at the time of binding. After binding and washing, the antibody is labeled in order to render it detectable.

Protein Arrays. The CD44-binding ligand can also be immobilized on a protein array. The protein array can be used as a diagnostic tool, e.g., to screen medical samples (such as isolated cells, blood, sera, biopsies, and the like). Of course, the protein array can also include other ligands, e.g., that bind to CD44 or to other target molecules, such as hyaluronic acid.

Methods of producing polypeptide arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

For example, the array can be an array of antibodies, e.g., as described in De Wildt, supra. Cells that produce the protein ligands can be grown on a filter in an arrayed format. Polypeptide production is induced, and the expressed polypeptides are immobilized to the filter at the location of the cell.

A protein array can be contacted with a labeled target to determine the extent of binding of the target to each immobilized polypeptide from the diversity strand library. If the target is unlabeled, a sandwich method can be used, e.g., using a labeled probed, to detect binding of the unlabeled target.

Information about the extent of binding at each address of the array can be stored as a profile, e.g., in a computer database. The protein array can be produced in replicates and used to compare binding profiles, e.g., of a target and a non-target. Thus, protein arrays can be used to identify individual members of the diversity strand library that have desired binding properties with respect to one or more molecules.

FACS. (Fluorescent Activated Cell Sorting). The CD44-binding ligand can be used to label cells, e.g., cells in a sample (e.g., a patient sample). The ligand is also attached (or attachable) to a fluorescent compound. The cells can then be sorted using fluorescent activated cell sorted (e.g., using a sorter available from Becton Dickinson Immunocytometry Systems, San Jose Calif.; see also U.S. Pat. Nos. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample.

The sorter can also deflect the cell and separate cells bound by the ligand from those cells not bound by the ligand. The separated cells can be cultured and/or characterized.

In vivo Imaging. In still another embodiment, the invention provides a method for detecting the presence of a CD44-expressing cancerous tissues in vivo. The method includes (i) administering to a subject (e.g., a patient having a cancer or neoplastic disorder) a CD44-binding antibody, conjugated to a detectable marker; (ii) exposing the subject to a means for detecting said detectable marker to the CD44-expressing tissues or cells. For example, the subject is imaged, e.g., by NMR or other tomographic means.

Examples of labels useful for diagnostic imaging include radiolabels such as ³¹¹I , ¹¹¹In, ¹²³I, ^{99m}Tc, ³²P, ¹²⁵I , ³H , ¹⁴C , and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The protein ligand can be labeled with such reagents using known techniques. For example, see Wensel and Meares (1983) Radioimmunoimaging and Radioimmunotherapy, Elsevier, New York for techniques relating to the radiolabeling of antibodies and D. Colcher et al. (1986) Meth. Enzymol. 121: 802-816.

A radiolabeled ligand-labelled CD44-binding antibody can also be used for in vitro diagnostic tests. The specific activity of a isotopically-labeled ligand depends upon the half-life, the isotopic purity of the radioactive label, and how the label is incorporated into the antibody.

Procedures for labeling polypeptides with the radioactive isotopes (such as ¹⁴C, ³H, ³⁵S, ¹²⁵I, ³²p, ¹³¹I) are generally known. For example, tritium labeling procedures are described in U.S. Pat. No. 4,302,438. lodinating, tritium labeling, and ³⁵S labeling procedures, e.g., as adapted for murine monoclonal antibodies, are described, e.g., by Goding, J. W. (Monoclonal antibodies:principles and practice:production and application of monoclonal antibodies in cell biology, biochemistry, and immunology 2nd ed. London ; Orlando: Academic Press, 1986. pp 124-126) and the references cited therein. Other procedures for iodinating polypeptides, such as antibodies, are described by Hunter and Greenwood (1962) Nature 144:945, David et al. (1974) Biochemistry 13:1014-1021, and U.S. Pat. Nos. 3,867,517 and 4,376,110. Radiolabeling elements which are useful in imaging include ¹²³I, ¹¹³I, ¹¹In, and ^{99m}Tc, for example. Procedures for iodinating antibodies are described by Greenwood, F. et al. (1963) Biochem. J. 89:114-123; Marchalonis, J. (1969) Biochem. J. 113:299-305; and Morrison, M. et al. (1971) Immunochemistry 289-297. Procedures for ^{99m}Tc-labeling are described by Rhodes, B. et al. in Burchiel, S. et al. (eds.), Tumor Imaging: The Radioimmunodichemical Detection of Cancer, New York: Masson 111-123 (1982) and the references cited therein. Procedures suitable for ¹¹¹In-labeling antibodies are described by Hnatowich, D. J. et al. (1983) J. Immul. Methods, 65:147-157, Hnatowich, D. et al. (1984) J. Applied Radiation, 35:554-557, and Buckley, R. G. et al. (1984) F.E.B.S. 166:202-204.

In the case of a radiolabeled ligand, the ligand is administered to the patient, is localized to the tumor bearing the antigen with which the ligand reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments are used to generate an image. However, these differences can be insufficient to provide sharp high resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., Fe⁺³ Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like

The CD44-binding ligands can also be labeled with an indicating group containing of the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image cancerous tissues.

Information obtained from evaluating a CD44-binding ligand, e.g., a ligand described herein, can be store as a computer representation, e.g., in a database, e.g., a database of images for one or a plurality of subjects. The database can be a relational database. The term “computer representation” refers to information which is in a form that can be manipulated by a computer. The act of storing a computer representation refers to the act of placing the information in a form suitable for manipulation by a computer.

Kits

Also within the scope of the invention are kits that include a composition described herein, e.g., a composition that contains a CD44-binding protein. In one embodiment, the kit includes (a) a composition that includes the CD44-binding protein, and, optionally, (b) informational material. The informational material can be descriptive, instructional, marketing or other material that relates to the methods described herein and/or the use of the compound for the methods described herein, e.g., a treatment, prophylactic, or diagnostic use. For example, the informational material describes methods for administering the composition to treat a disorder, e.g., a neoplastic disorder or an inflammatory disorder, or a disorder characterized by excessive CD44activity.

In another example, the composition includes a CD44-binding cell agonist or a CD44-binding cell sensitizing agent. The informational material can describes methods for administering the composition to sensitize a cell, e.g., an NK cell or to prepare a subject to receive exogenous cells, e.g., allogenic, xenogenic, matched, or unmatched cells.

In one embodiment, the informational material can include instructions to administer the compound in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., a human having, or at risk for a disorder characterized by excessive elastase activity. The informational material can include information about production of the compound, molecular weight of the compound, concentration, date of expiration, batch or production site information, and so forth. The informational material of the kits is not limited in its form. Information about the compound can include structural information, e.g., amino acid sequence, tradename, FDA approved name, antibody isotype, and so forth. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the compound and/or its use in the methods described herein. The informational material can also be provided in any combination of formats.

In addition to the composition that includes the CD44-binding protein, the composition itself can include other ingredients, such as a solvent or buffer, a stabilizer or a preservative, and/or a second agent for treating a condition or disorder described herein, e.g. a neoplastic or inflammatory (e.g., IBD or RA) disorder. Alternatively, such other ingredients can be included in the kit, but in different compositions or containers than the composition that includes the CD44-binding protein. In such embodiments, the kit can include instructions for admixing the compound and the other ingredients, or for using the compound together with the other ingredients.

The composition that includes the CD44-binding protein can be provided in any form, e.g., liquid, dried or lyophilized form. It is preferred that composition be substantially pure and/or sterile. When the composition that includes the CD44-binding protein is provided in a liquid solution, the liquid solution preferably is an aqueous solution, with a sterile aqueous solution being preferred. When the composition that includes the CD44-binding protein is provided as a dried form, reconstitution generally is by the addition of a suitable solvent. The solvent, e.g., sterile water or buffer, can optionally be provided in the kit.

The kit can include one or more containers for the composition that includes the CD44-binding protein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the CD44-binding protein. For example, the kit includes a plurality of syringes, ampules, foil packets, or blister packs, each containing a single unit dose of the compound. The containers of the kits can be air tight, waterproof (e.g., impermeable to changes in moisture or evaporation), and/or light-tight.

Kits can be provided that include a CD44-binding antibody and instructions for diagnostic, e.g., the use of the CD44-binding ligand (e.g., antibody or antigen-binding fragment thereof, or other polypeptide or peptide) to detect CD44, in vitro, e.g., in a sample, e.g., a biopsy or cells from a patient having a cancer or neoplastic disorder, or ill vivo, e.g., by imaging a subject. The kit can further contain a least one additional reagent, such as a label or additional diagnostic agent. For in vivo use the ligand can be formulated as a pharmaceutical composition.

The following invention is further illustrated by the following non-limiting examples.

### EXAMPLES

### Example 1

Selection and Primary Screening

Antibodies that recognize activated CD44 were selected from Dyax Corp.'s CJ phagemid library for binding to neuraminidase treated, soluble, biotinylated-CD44Fc protein immobilized on streptavidin magnetic beads.

Construction of Soluble CD44Fc

Human blood collected in the presence of sodium heparin was spun down to isolate the mononuclear cell layer. Cells were washed and mixed with RNAZOL™ (Cinna Bioteckx Laboratories) to isolate the RNA, as described in the product insert. Following cDNA production using poly dT priming, the extracellular domain of CD44 and the Fc portion of human IgG, were amplified. The extracellular domain of CD44 was cloned upstream of the Fc domain with a Factor X site in between. This construct, named CD44Fc, was cloned into pIRESneo2 (Clontech) to allow expression of soluble CD44Fc in mammalian cells.

Expression and Purification of CD44Fc

HEK293T cells were transfected with endotoxin free DNA (Qiagen) (pIRESneo2CD44Fc) by standard methods using LIPOFECTAMINE™ 2000 (Invitrogen). Supernatants were harvested after 72 and 144 hours. Soluble CD44Fc was then purified from the pooled cell culture supernatant by immobilized Protein A chromatography and characterized by protein staining using SDS-PAGE, Western Blot and ELISA methods.

CD44Fc Biotinylation

Purified CD44-Fc was reacted with a 5-fold molar excess of sulfo-NHS—SS-biotin in 50 mM HEPES, pH 8.0, 100 mM NaCl overnight at 4° C. Free biotin was removed by buffer exchange into PBS, 0.01% Tween 20 using a BIOMAX™ device with a 10 kDa molecular weight cut-off membrane. Protein was quantified by absorbance at 280 nm where 1 mg/mL =1.039 OD. The number of biotin molecules incorporated per mole of CD44-Fc was determined using the HABA assay as described by the manufacturer (Pierce).

Binding of Hyaluronic Acid (HA) to Immobilized CD44Fc:

Biotinylated CD44Fc (b-CD44Fc) was treated with V. cholerae neuraminidase (Roche), which was chosen for its broad substrate specificity. Other glycosidase enzymes could have been used providing that, empirically, their activity could produce CD44 (e.g., b-CD44Fc) having increased affinity for HA (e.g., FL-HA). Briefly, 200 μg of CD44Fc plus 0.2 units neuraminidase per ml of PBS were incubated for 18 hours at 37° C. A control sample of b-CD44Fc was treated under identical conditions except without neuraminidase. Dilutions of neuraminidase treated and control b-CD44Fc were mixed with streptavidin coated polystyrene particles (Spherotech) for 1 hour at room temperature, blocked with 10% horse serum in DMEM and washed in HEPES/DMEM with 2% fetal calf serum. The b-CD44Fc coated beads were then incubated with Fluorescein HA (FL-HA), CD44-binding-FITC antibody, or various lectins for 45 minutes on ice. After a few washes, the HA binding activity, relative concentration of CD44 and sialic removal was determined by flow cytometric analysis on a FACSCANT™ (Becton Dickinson). Fluorescein-labeled HA was prepared following the isocyanide procedure as described in De Belder and Wik, Preparation and properties of fluorescein-labeled hyaluronate, Carbohydr Res 44(2):251-7 (1975).

Selection and Screening for CD44-Binding Antibodies:

Using Dyax Corp.'s CJ phagemid Fab library, 3 rounds of selection were done with neuraminidase treated b-CD44Fc (100, 50 and 50 μg for 1st, 2nd and 3rd round respectively) immobilized on streptavidin coated magnetic beads (Dynal). The library was depleted against streptavidin coated magnetic beads on each round of selection, and soluble Trail-Fc (a commercially available Fc fusion protein) was included during the selections to remove Fc binders from being selected. Each round of selection included two cycles of streptavidin magnetic bead depletion, a cycle of binding of phage to treated b-CD44Fc coated beads, ten cycles of washes, elution of bound phage, and propagation of enriched phage ready for the next round. Phage bound to biotinylated-CD44Fc coated beads after ten washes were directly amplified, or eluted with 200 μg/mL of HA before amplification. After three rounds of selection, individual clones were grown in 96-well microtiter plates and were screened for CD44 binding activity by phage ELISA, using biotinylated-CD44Fc, Trail-Fc or streptavidin as targets. Numerous isolates (85%) showed reactivity to biotinylated-CD44Fc, but not to Trail-Fc or streptavidin. These isolates were DNA fingerprinted to determine their diversity. More than 10 different clones were identified.

ELISAs:

Individual clones were grown and rescued as described previously (Marks et al. (1991), J. Mol. Biol. 222:581). For the CD44Fc ELISAs, 96-well IMMULON2 HB™ plates (Thermo Labsystems) were coated with 1 μg/well IMMUNOPURE™ streptavidin (Pierce) in PBS and incubated overnight at 4° C. After three washes with PBS, 100 μL of the b-CD44Fc was added and allowed to bind to streptavidin for 30-60 minutes at room temperature. Next, the b-CD44Fc coated wells were blocked with 300 μL of 2% milk/1×PBS/0.05% Tween (2% MPBST) for two hours at 37° C. The b-CD44Fc coated wells were then incubated with 100 μL of phage culture supernatant that had been blocked with 2% MPBST for one hour at room temperature, washed five times with 1×PBS/Tween 0.1% (PBST), and incubated with 100 μL of anti-M13-HRP secondary antibody at a 1:5,000 dilution for one hour at room temperature. The b-CD44Fc coated wells were washed five times with PBST before developing with TMB-solution and read at 630 nm.

For the cell ELISAs, cells were washed once in PBS and resuspended at a concentration of 1×10⁶ to 2×10⁶ cells/mL of PBS. A final concentration of 1-2×10⁵ cells per well of a 96-well tissue culture plate (Falcon, VWR) was used. The cells were fixed by adding an equal volume of 0.2% glutaraldehyde (Sigma-Aldrich) and incubating at 37° C. for 12 minutes. They were then washed three times with PBS using an automated plate washer (Bio-Tek Instuments, Inc.) and blocked with 200 μL of 2% MPBST for one hour at room temperature. The rest of the ELISA was performed as described above except that 1×PBS/Tween 0.05% was used for the washes and incubations. For the HA inhibition assay, the wells were incubated with 50 μL of blocked phage culture supernatant, followed by overlaying 50 μL of 0.1 μg/ml HA-FITC per well and incubation for another hour at room temperature. After five washes with PBST, the samples were incubated with 100 μL of anti-FITC-HRP secondary antibody (Dako) at 1:800 dilution and incubated for one hour. Alternatively, for the OS/37 Ab competition ELISA, the samples were incubated with 50 μL of the CD44-binding OS/37 antibody (Seikagaku Corporation) at a 1:400 dilution in 2% MPBST for one hour at room temperature, followed by overlaying with 50 μL of blocked phage culture supernatant and incubation for an additional hour at room temperature.

DNA Fingerprinting of Clones:

Positive isolates were PCR amplified with the oligonucleotide primers M13-reverse and geneIII-forward (Marks et al. (1991), J. Mol. Biol. 222:581), and the product analyzed by BstNI fingerprinting.

### Example 2

Secondary Screening Against CD44 Expressed on Cultured Cells

To verify the binding of selected clones to CD44 expressed on cells, the monocytic cell line KG1a was utilized. Cell ELISAs, as described above, were done using untreated and neuraminidase treated KG1a cells. V. cholerae neuraminidase treated KG1a cells gain higher affinity for HA, as if CD44 has been activated.

The KG1a gain in affinity for HA was confirmed by flow cytometry. KG1a cells were incubated with 10 μL CD44-binding-FITC antibody (clone FI0-44-2, Biosource), or 10 μg/mL FL-HA for 20 minutes on ice. After two washes with PBS, the cells were fixed with 2% paraformaldehyde (Sigma-Aldrich) in PBS, read in a FACS scan flow cytometer (FACSCAN™, BD Biosciences) and analyzed using CELL QUEST™ software (BD Biosciences). For T cells or monocytes, 1×10⁶ of the isolated cells were incubated with 200 μL of heat-inactivated autologous plasma for 10 minutes on ice and washed once in PBS before staining for analysis.

Treatment with neuraminidase increased the binding capacity of CD44 to HA by 84%. There was no change in CD44 expression after treatment. Furthermore, ELISAs performed with these cells showed that several clones bound significantly more to neuraminidase treated KG1a cells than to mock treated cells. B12 is the CD44-binding control and F12 is the HA-FITC control.

Some clones competed directly with HA for binding to neuraminidase treated KG1a cells. FL-HA was incubated with neuraminidase-treated KG1a cells, followed by competition with Fab displaying phage. A number of phage isolates inhibited HA binding in this assay relative to controls.

All cells were grown at 37° C. in a 0.5% CO₂ incubator. The monocyte cell line KG1a was grown in Iscove's modified Dulbecco's medium plus 20% fetal bovine serum and 4 mM L-glutamine and 1.5 g/L sodium bicarbonate. KG1a cells were treated with neuraminidase from V. cholerae (Roche). Briefly, KG1a cells were washed once in a 1:1 solution of RPMI/PBS and resuspended at 10⁷ cells/mL of RPMI/PBS containing 0.05 units/mL of neuraminidase for 75 minutes. Control cells were treated under identical conditions except without neuraminidase, then they were washed twice in PBS.

### Example 3

Screening Against CD44 Expressed on Primary Leukocytes

Since activation of CD44 binding to HA in vivo can be accomplished through several mechanisms, we tested the ability of the isolated phage clones to recognize activated CD44 as expressed on the surface of leukocytes. In order to activate CD44 on primary T cells, peripheral blood mononuclear cells (PBMCs) were isolated from whole blood and cultured in the presence or absence of PMA and ionomycin for 18 hours. T cells were isolated by magnetic depletion of the non-T cell population. FACS analysis confirmed activation of CD44 in the T cell population. There was a 44% increase in FL-HA binding by T cells after treatment. As published previously in Brown et al. (2001), J. Immunol 167(9):5367-74; Maiti et al. (1998), Science 282:941-3, there was also a 14% increase in CD44 expression in these cells. When the same CD44-binding antibody (clone F10-44-2) was used in the ELISA, the difference in CD44 signal was not significantly different between the PMA/ionomycin treated cells and the untreated controls. Several Fab-expressing phage clones showed an increased signal to activated T cells when compared to unstimulated PBMC cells.

The PBMC isolation and culture was done as described previously (Brown et al. (2001), J. Immunol 167(9):5367-74). Briefly, whole blood (200-400 mL) from healthy volunteers was collected and treated with sodium heparin. The PBMCs were separated by centrifugation over a FICOLL-PLAQUE PLUS™ (Amersham Biosciences) density gradient. Contaminating red blood cells in the buffy coat were lysed using a human erythrocyte lysis kit from R&D Systems. The PBMCs were cultured at 2.5×10⁶ cells/mL in RPMI 1640/10% FBS with or without the following treatments: 500 units/mL IFN-γ (R&D Systems) for 72 hours, or 1 ng/mL PMA plus 500 ng/mL ionomycin (both from Sigma-Aldrich) for 18 hours. Cells were seeded into 6-well ultra low attachment plates (Corning, VWR) for all applications except for stimulation of the T cell population with PMA and ionomycin where they were seeded into 6-well tissue culture plates (Falcon, VWR). After incubation the T cells or the CD14-positive monocyte/macrophage populations were isolated using the Pan T cell Isolation Kit or anti-CD14 MICROBEADS™, respectively (Miltenyi Biotech).

PBMCs were also stimulated with IFN-gamma for 72 hours, after which the monocyte/macrophage population was isolated using anti-CD14 MICROBEADS™. This population had an increase in FL-HA binding of 75% when compared to non-stimulated cells, as determined using FACS analysis. The CD44 levels did not change significantly after stimulation as detected by this method. ELISA results demonstrated a similar increase in FL-HA binding to activated CD14-positive cells, while the CD44 expression levels were not significantly different. Phage reactivity to the activated monocytes/macrophages was moderately increased as compared to their reactivity towards unstimulated PBMCs.

### Example 4

Screening Against Murine CD44

We analyzed the reactivity of the CD44 binding clones to murine CD44 as expressed in a surface lymphocyte cell line. ELISA reactivity to BW5147.3, which expresses constitutively active CD44, was compared to the CD44-negative murine cell line AKR-1. Several clones show species cross reactivity.

All cells were grown at 37° C. in a 0.5% CO₂ incubator. Mouse cell line BW5147.3 was grown in DMEM plus 10% horse serum and I mM sodium pyruvate. The mouse cell line AKR-1 was grown in DMEM (Mediatech) plus 10% horse serum.

### Example 5

Screening of Soluble Fabs Against CD44 Expressed on Cultured Human Cells and Screening for Inhibition of HA Binding

Four Fabs—A2, A3, G2, H10—were tested for their ability to bind to human CD44+ cells and their ability to inhibit hyaluronan (HA) binding. The A2 Fab is a negative control.

Methods

Binding by the Fabs was analyzed as follows:

1. Cells incubated with varying concentrations of Fabs at 4° C. for 15-20 min.

2. washed once

3. incubated with a detection antibody (either anti-cmyc or anti-6His) for 20 min at 4° C.,

4. washed 1×

5. incubated with a secondary antibody: goat anti-mouse (G×M) FITC antibody for 20 min at 4° C.

6. washed twice

7. analyzed on a FACSCAN™ machine (Becton Dickinson).

Fabs were detected with (1) a murine anti-cmyc tag antibody (9E10) and G×M FITC secondary antibody; or (2) a murine anti-6His mAb and G×M FITC secondary antibody. Later experiments predominantly used the anti-6His antibody because its signal was better. A mouse anti-human CD44 mAb that inhibits HA binding was used as a positive control. Binding by this monoclonal could be directly detected with the G×M FITC secondary antibody.

HA binding and inhibition was assessed by binding fluorescently labeled HA (F1-HA) to cells in the presence or absence of Fabs. FL-HA binding was also detected using FACS. Binding data are represented graphically as either amount of fluorescence (FL-1, arbitrary units) or as % inhibition of FL-HA binding.

Results. At low concentrations (0.1-1 μg/mL), the Fabs demonstrated low binding (Table 4) to KG1a cells and no inhibition of HA binding (Table 5).

TABLE 4
Fab Binding at Low Concentration

| Antibody | FL Counts* |
|---|---|
| Positive Control | 272 |
| A2 | 6 |
| A3 | 9 |
| G2 | 10 |
| H10 | 9 |

*Values are FL counts in arbitrary units.

TABLE 5
Inhibition of HA binding at Low Concentration

| Antibody | % Inhibition |
|---|---|
| Positive Control | 83.00 |
| A2 | 7.00 |
| A3 | 20.00 |
| G2 | 10.00 |
| H10 | 16.00 |

Increasing the concentration of Fabs to 10 and 20 μg/mL increased binding to KG1a (Table 6, columns 2 and 3) and SR91 cells (a human myelo-monocytic cell line) (Table 6, columns 4 and 5). Binding by the G2 Fab was particularly evident at these concentrations (values being at least 4 times background). Similar results were seen at higher concentrations (Table 7).

TABLE 6
CD44+ Cell Binding at Intermediate Concentrations (I)

| | KG1a-HA + Cells | | SR91 Cells | |
|---|---|---|---|---|
| Antibody | 10 μg/mL | 20 μg/mL | 10 μg/mL | 20 μg/mL |
| A2 | 2.68 | 3.53 | 2.09 | 2.24 |
| A3 | 2.74 | 3.98 | 2.33 | 1.94 |
| G2 | 9.37 | 15.48 | 4.35 | 9.1 |
| H10 | 2.88 | 3.46 | 2.2 | 2.42 |
| 2° Ab control | 2.08 | | 2.08 | |

*Values are FL counts in arbitrary units.

Dose dependent Fab binding and dose dependent inhibition of HA binding was observed for the G2 Fab at concentrations between 25 and 100 μg/mL. See Table 7 and Table 8.

TABLE 7
CD44+ Cell Binding at Intermediate Concentrations (II)

| Antibody | 25 μg/mL | 50 μg/mL | 100 μg/mL |
|---|---|---|---|
| 9E10 + gxm | 29 | 29 | 29 |
| A2 | 30 | 32 | 34 |
| A3 | 33 | 31 | 33 |
| G2 | 46 | 72 | 89 |
| H10 | 34 | 30 | 32 |

*Values are FL counts in arbitrary units.

TABLE 8
HA Blocking by Fabs at Varying Concentrations

| Antibody* | 25 μg/mL | 50 μg/mL | 100 μg/mL |
|---|---|---|---|
| HA | 161 | | |
| Positive Control | 20 | | |
| A2 | 199 | 171 | 181 |
| A3 | 174 | 173 | 149 |
| G2 | 146 | 117 | 93 |
| H10 | 198 | 185 | 145 |

*Except row 1 (HA).

Similar results were obtained for binding in assays that used the 6HIS mAb for detection of Fab binding. See Table 9. G2 bound in a dose dependent manner at concentrations between 20-100 μg/mL. H10binding was observed at 100-1000 μg/mL.

KG1a cells were selected for hi HA binding (HA+) or lo HA binding (HA−). The lo HA binding cells express lower levels of CD44, but still bind significant levels of HA. Binding correlated with the level of CD44 as less binding was observed low CD44-expressing cells.

TABLE 9
Fab Binding using anti-6His Ab Detection

| Fab: | G2 Fab | | H10 Fab | |
|---|---|---|---|---|
| Conc. in μg/mL | KG1a-HA+ | KG1a-HA− | KG1a-HA+ | KG1a-HA− |
| (anti-6His/ | 6 | 6 | 6 | 6 |
| gxm-FITC) | | | | |
| 20 | 43 | 48 | 9 | 7 |
| 50 | 134 | 99 | 12 | 12 |
| 100 | 240 | 213 | 39 | 34 |
| 1000 | | | 108 | 132 |

The results in Table 10 indicated that G2 binding to KG1a hi HA binding cells saturated at 125 μg/mL, but is dose dependent for KG1a HA lo selected cells. H10 binding was dose dependent for both cell types.

Both G2 and H10exhibited significant inhibition of HA binding, although the H10 Fab did not bind as well as the G2 Fab. However, direct comparison of binding of Fabs with positive control mAb was not possible in this assay since the control mAb was detected using G×M FITC and whereas the Fabs were detected by anti-6His and G×M FITC.

G2 inhibited the binding of HA to CD44+ KG1a cells to 60-80% inhibition at 100 μg/mL (Table 11). The H10Fab exhibited significant inhibition (˜60%) at concentrations above 250 μg/mL. These results indicated that G2 has a higher affinity for CD44 than H10. With undiluted TCS, ˜90% inhibition was observed for the positive control antibody whereas maximal inhibition by the G2 Fab was ˜80%.

TABLE 10
Binding Titration

| | KG1a-HA+ | | | | KG1a-HA− | | | |
|---|---|---|---|---|---|---|---|---|
| | 125 μg/mL | 250 μg/mL | 500 μg/mL | 1 mg/mL | 125 μg/mL | 250 μg/mL | 500 μg/mL | 1 mg/mL |
| 2° Ab control | 7 | 7 | 7 | 7 | 7 | 7 | 7 | 7 |
| Positive C | 289 | 328 | 448 | 332 | 331 | 345 | 407 | 352 |
| G2 | 872 | 629 | 619 | 864 | 387 | 521 | 674 | 846 |
| H10 | 65 | 92 | 106 | 176 | 46 | 74 | 108 | 117 |

Values are arbitrary fluorescence units.
The concentration of Fabs is 125-1000 μg/ml as listed below the cell type, and the concentration for the positive control is (from left to right) 1/8, 1/4, 1/2, or 1/1 dilution of tissue culture supernatant.

TABLE 11
Titration for HA Inhibition

| | KG1a-HA+ | | | | KG1a-HA− | | | |
|---|---|---|---|---|---|---|---|---|
| | 125 μg/mL | 250 μg/mL | 500 μg/mL | 1 mg/mL | 125 μg/mL | 250 μg/mL | 500 μg/mL | 1 mg/mL |
| Positive C | 32.7 | 58.9 | 85.0 | 94.4 | 73.8 | 52.9 | 32.0 | 24.5 |
| G2 | 66.4 | 67.3 | 72.0 | 77.6 | 46.9 | 46.2 | 42.4 | 37.9 |
| H10 | 11.2 | 48.6 | 57.0 | 65.4 | 91.0 | 61.1 | 54.4 | 47.7 |

Values are % inhibition of HA binding.
the conc. of FABS is 125-1000 μg/mL and the conc. for the positive control is 1/1-1/8 dilution of tissue culture supernatant (TCS)

### Example 6

Affinity of Fabs and IgGs for CD44

Divalent IgG4 antibodies (Abs) were produced using the full light chain and heavy chain variable region of the Fabs A2, A3, G2 and H10. The affinities of the Fabs and the corresponding divalent IgG4 Abs for human CD44-Fc and neuraminidase-treated (activated) CD44-Fc were evaluated in BIACORE™ analysis. The data are summarized in Table 13. For the divalent IgG4 Abs except H10, increased affinities relative to the parental Fabs were observed.

TABLE 12
Antibody Affinities for CD44

| | |
|---|---|
| CD44-binding | K_{D} (nM) |

| Antibody | CD44-Fc | CD44-Fc(Activated) |
|---|---|---|
| A2 Fab | 2820 | 249 |
| A2 IgG4 | 2 | 1.5 |
| A3 Fab | >500 | >500 |
| A3 IgG4 | 125 | 131 |
| G2 Fab | 239 | 353 |
| G2 IgG4 | 123 | 173 |
| H10 Fab | 487 | 388 |
| H10 IgG4 | 2348 | 3448 |

### Example 7

Titration of IgGs Against CD44 Expressed on Cultured Human Cells and Titration for Inhibition of HA Binding

Divalent IgG4 antibodies (Abs) A2, A3, G2 and H10were produced. Human myeloid cells (KG1a) were incubated with different concentrations (100, 75, 50, 12.5, 6.2, 3.1, 1.5, 0.75, 0.37, 0.18, 0.09, 0.04, 0.02, 0.01, 0.005 μg/mL) of A2, A3 or G2 antibodies on ice for 20 min or with 400, 200,100, 75, 50, 12.5, 6.2, 3.1, 1.5, 0.75, 0.37, 0.18, 0.09, or 0.04 μg/mL of A2 or H10Abs on ice for 20min. Cells were washed once then incubated with secondary antibodies, goat anti human-FITC (Jackson Immune Research) 1/50 dilution for A2, A3, G2 and H10antibodies or goat anti mouse-FITC (Southern Biotech) 1/50 dilution for a positive control antibody for 20 min. on ice. Cells were washed once and then analyzed on FACSCAN™. Results are summarized in Table 13. Because the positive control was a mouse anti-human Ab and a different secondary Ab was used to detect it, the highest mean fluorescence values for each antibody were converted to 100 and other values were calculated as a percentage of the highest value. Each graph depicts an average of three or four experiments.

HA binding inhibition was assessed by adding different concentrations of antibodies or PBS in control wells to cells incubated for 10 min. on ice, then HA-FITC 1/100 dilution was also added and incubated for 20 min. Cells were washed once, then binding and blocking of HA-FITC to cells was detected using FACSCAN™. Results are summarized in Table 13. Using the 50% binding concentrations of the antagonist IgG4s for human CD44 expressed on KG1a cells to calculate estimated IC₅₀ values, the IC50s are as follows: HAE-A3 IgG4: 4.2×10e-9 M; HAE-G2 IgG4: 1.0×10e-9 M; HAE-H10IgG4: 6.9×10e-8 M; Positive control mouse anti-human IgG: 4.2×10e-9M. Accordingly, antibodies with IC50's less than 200, 100, 50, 10, or 5 nM can be used.

TABLE 13
CD44 Binding and HA Inhibition Titration

| | | | 50% CD44 |
|---|---|---|---|
| | | Initial | Binding or |
| | | Saturation | HA Blocking |
| IgG | | Conc. | Conc. |
| Tested | Titration parameter | (μg/mL) | (μg/mL) |
| A3 IgG4 | Binding to CD44 on cells | 25 | 0.75 |
| | Block HA binding to CD44 on | 6 | 0.75 |
| | cells | | |
| G2 IgG4 | Binding to CD44 on | 0.75-1.5 | 0.18 |
| | cells | | |
| | Block HA binding to CD44 on | 0.75 | 0.18 |
| | cells | | |
| Positive | Binding to CD44 on | 12.5 | 0.75 |
| control | cells | | |
| M anti-H | Block HA binding to CD44 on | 6 | 0.5 |
| IgG | cells | | |
| H10 IgG4 | Binding to CD44 on | 100 | 12.5 |
| | cells | | |
| | Block HA binding to CD44 on | 50 | 6.2 |
| | cells | | |
| Positive | Binding to CD44 on cells | 12.5 | 0.75 |
| control | Block HA binding to CD44 on | 6 | 0.2 |
| M anti- | cells | | |
| H IgG | | | |
| Negative | Binding to CD44 on | No binding | No blocking |
| control | cells | | |
| A2 IgG4 | Block HA binding to CD44 on | No binding | No blocking |
| | cells | | |

### Example 8

Assay of IgGs for Binding to Human Endothelial Cells and T Cells and Assay for Inhibition of HA Binding

The binding of IgG4 Abs to human endothelial cells and CD44-transfected Jurkat T cells was determined by FACS analysis. Inhibition of HA binding by these cells was determined by measuring the ability of the antibodies to block binding of fluoresceinated hyaluronan (HA-FITC).

Human cells (2×10⁵) in 50 μL volume were incubated with saturating concentrations (as determined for KG1a in Example 7), or above, of each antibody for 20 min. on ice, i.e., 50 μg/mL of A3 and A2, 6.2 μg/mL of G2 and 100 μg/mL of H10. In some cases, these amounts were titrated to examine binding and % HA inhibition. Cells were washed 1× then incubated with fluoresceinated secondary antibodies: 1/50 goat anti-human Ig FITC (Jackson Immune Research), or 1/50 goat anti-mouse Ig FITC (Southern Biotech) for 20 minutes on ice. Cells were washed once and then analyzed on FACSCAN™ using CELL QUEST™ software (BD Biosciences). HA binding and inhibition was assessed by adding different concentrations of antibodies or PBS in control wells to cells incubated for 10 minutes on ice, then 50 μL 1/100 dilution of HA-FITC was added and incubated for 20 min. on ice. Cells were washed once then binding of HA-FITC to cells was detected by FACSCAN™.

Results: For the cell types assayed, levels of fluorescence between the control anti-human CD44 monoclonal antibody (HuCON) and the IgG4 antibodies are not necessarily comparable since different secondary detection antibodies were used.

Human Microvascular Endothelial Cell Line (HMEC)

HMECs express human CD44 but have low binding capacity for HA. All IgG4 antibodies except A2 bound well to these cells: A3 at 50, 25, and 12.5 μg/mL; G2 at 6.2, 3.1, and 1.5 μg/mL; H10 at 100, 50, and 25 μg/mL. The positive control monoclonal antibody bound well at 12.5, 6.2 and 3.1 ∥g/mL. The antibodies A3, G2, H10 and the positive control all partially reduced HA binding. As HA binding by HMEC was poor, it was difficult to get a precise value both for HA binding and % inhibition with these cells, however, A3, G2 and H10acted like the positive control.

Human Umbilical Vein Endothelial Cell Line (HUVEC)

HUVEC express human CD44 and have low to medium binding capacity for HA. All IgG4 antibodies except A2 bound well to these cells: A3 at 50 μg/mL; G2 at 6.2 μg/mL; and H10 at 100 μg/mL. The antibodies G2, A3, H10 and the positive control substantially reduced the ability of HUVEC cells to bind to HA.

Human Jurkat T Cell Line

Jurkat T cells do not express human CD44 and do not bind HA. The IgG4 antibodies that bind CD44, like the positive control, did not bind to this CD44-negative cell line further substantiating that the antibodies are specific for human CD44.

Human Jurkat T Cells Transfected with Human CD44-H or CD44-R1

These transfected cell lines express either the isoform of human CD44 that predominates on resting cells (CD44-H) or the isoform that is predominantly present on the myeloid cell line KG1a (CD44-R1). Neither of these transfected lines bind significant levels of HA. All IgG4 antibodies except A2 bound to these cells. For example, A3 bound at concentrations of 50, 25, and 12.5 μg/mL; G2 at 6.2, 3.1, and 1.5 μg/mL; and H10 at 100, 50, and 25 μg/mL. The positive control monoclonal antibody bound at 12.5, 6.2 and 3.1 μg/mL. The G2 antibody was the best binder.

Human Jurkat T Cells Transfected with Human CD44 Point Mutant

This transfected cell line expresses an isoform of human CD44 that binds HA to a greater extent than the CD44-H or CD44-R1 isoforms. All IgG4 antibodies except A2 bound well to these cells and substantially inhibited HA binding: A3 at 50 μg/mL; G2 at 6.2 μg/mL; H10 at 100 μg/mL.

Overall Conclusions for Binding to Human Cells

Lack of binding to CD44-negative Jurkat T cells and binding to CD44-transfected Jurkat T cells demonstrates that Abs A3, G2 and H10 are specific for human CD44. These antibodies bind to CD44 expressed on human cells, irrespective of HA binding ability and bind to both CD44-H and CD44-R1 isoforms. When cells bind HA in CD44-dependent manner, antibodies A3, G2 and H10, like the positive control antibody, can substantially inhibit HA binding. Though antibody A2 binds CD44-Fc in BIACORE™ analysis (Example 6), it does not bind to CD44+ or CD44− human cells.

### Example 9

To identify CD44 binding clones that potentially recognize the overlapping CD44 epitopes bound by the S5 mouse anti-canine CD44 monoclonal antibody and the IM7 rat CD44-binding monoclonal antibody, phage isolates that showed reactivity to biotinylated-CD44Fc were screened by competition phage ELISA, a modification of the phage ELISA of Example 1. The b-CD44Fc coated wells were incubated with 450 ng of S5 or IM7 antibody in 100 μl of 2% MPBST for one hour at room temperature, then 50 μl of phage culture supernatant was added for an additional incubation period of one hour at room temperature. Wells were then washed, incubated with anti-M13-HRP antibody, washed again and developed with TMB-solution as described in Example 1. Clones that were blocked from binding b-CD44Fc by either the S5 or IM7 antibody or both (Table 14) underwent DNA fingerprinting and sequencing analysis and several unique candidate agonist isolates were identified.

TABLE 14
Blocking by S5 Monoclonal Antibody

| | CD44 + S5 | CD44 |
|---|---|---|
| BE-A11 | 0.261 | 1.083 |
| BE-B12 | 0.303 | 0.785 |
| BE-D7 | 0.327 | 0.598 |
| BE-H10 | 0.359 | 1.081 |

Clones inhibited by IM7 include: BE-B12, BE-D7, BE-H10, BE-H9, BE-A11, HAE-B8, HAE-F1, HAE-H-H10. Clones inhibited by S5 include: BE-B12, BE-D7, BE-H10, BE-H9, BE-A1.

### Example 10

In vitro functional assays are also used to evaluate antibodies that recognize CD44. Exemplary assays include: 1. A static leukocyte-endothelial cell adhesion assay. The ability of antibodies to affect adherent cells in fluorescence microplate assay is evaluated. The analysis is used to determine the ability of antibodies to disrupt adherence between HMEC cells and KG1a cells. 2. Cell migration and wound healing assay. The ability of antibodies to modulate measured migration of cells (particularly endothelial cells after wound infliction) or in response to a particular signal is evaluated. 3. Cellular flow rolling and adhesion assay. Cells in medium are mixed with each antibody. The ability of each antibody to inhibit rolling on and adhesion to either hyaluronan or endothelial cells immobilized on a flow chamber surface is evaluated.

Other embodiments are within the following claims.

What is claimed:

1. An isolated protein comprising a light chain (LC) immunoglobulin variable domain sequence and a heavy chain (HC) immunoglobulin variable domain sequence,
wherein the LC and HC variable domain sequences form an antigen binding site with binding affinity for the human CD44 extracellular domain and wherein CDR3 of the LC variable domain sequence comprises M-Q-A-L-Q-X₁—P—X₂-T, where X₁ is threonine or absent, and X₂ is any amino acid or absent.

2. The protein of claim 1 wherein the LC variable domain sequence is a kappa light chain family member.

3. The protein of claim 1 wherein CDR2 of the LC variable domain sequence comprises an amino acid sequence of at least 6 amino acids of which at least 5 amino acids are identical to LGSNRAS, and
CDR1 of the LC variable domain sequence comprises an amino acid sequence of at least 15 amino acids of which at least 13 amino acids are identical to RSSQSLLHSNGYNYLD.

4. The protein of claim 3 wherein CDR2 of the HC variable domain sequence comprises: G-G-X₁-T—X₄—Y-A-D-S—V—K-G, where X₁ is hydrophobic, and X₄ is any amino acid.

5. The protein of claim 4 wherein each of CDR1, CDR2, and CDR3 of at least one the variable domain sequences differs by no more than two amino acid differences from a respective CDR of the HAE-A3 antibody.

6. The protein of claim 5 wherein CDR1, CDR2, and CDR3 of the HC and LC variable domain sequences are identical to respective CDRs of the HAE-A3 antibody.

7. The protein of claim 4 wherein each of CDR1, CDR2, and CDR3 of at least one the variable domain sequences differs by no more than two amino acid differences from a respective CDR of the HAE-G2 antibody.

8. The protein of claim 7 wherein CDR1, CDR2, and CDR3 of the HC and LC variable domain sequences are identical to respective CDRs of the HAE-G2 antibody.

9. The protein of claim 4 wherein each of CDR1, CDR2, and CDR3 of at least one the variable domain sequences differs by no more than two amino acid differences from a respective CDR of the HAE-H10antibody.

10. The protein of claim 9 wherein CDR1, CDR2, and CDR3 of the HC and LC variable domain sequences are identical to respective CDRs of the HAE-H10antibody.

11. The protein of claim 10 wherein FR3 of the HC variable domain sequence comprises: RFTISRDNSKNTLYLQMNSLRAEDTAVYYCAR.

12. The protein of claim 10 wherein FR3 of the HC variable domain sequence comprises: RFTISRDNSKNTLYLQMNSLRAEDTAVYHCAR.

13. The protein of claim 4 wherein each of CDR1, CDR2, and CDR3 of at least one the variable domain sequences differs by no more than two amino acid differences from a respective CDR of the BE-B12 antibody.

14. The protein of claim 13 wherein CDR1, CDR2, and CDR3 of the HC and LC variable domain sequences are identical to respective CDRs of the BE-B12 antibody.

15. The protein of claim 4 wherein each of CDR1, CDR2, and CDR3 of at least one the variable domain sequences differs by no more than two amino acid differences from a respective CDR of the BE-D7 antibody.

16. The protein of claim 15 wherein CDR1, CDR2, and CDR3 of the HC and LC variable domain sequences are identical to respective CDRs of the BE-D7 antibody.

17. The protein of claim 4 wherein each of CDR1, CDR2, and CDR3 of at least one the variable domain sequences differs by no more than two amino acid differences from a respective CDR of the BE-H10antibody.

18. The protein of claim 17 wherein CDR1, CDR2, and CDR3 of the HC and LC variable domain sequences are identical to respective CDRs of the BE-H10 antibody.

19. The protein of claim 4 wherein each of CDR1, CDR2, and CDR3 of at least one the variable domain sequences differs by no more than two amino acid differences from a respective CDR of the BE-H9 antibody.

20. The protein of claim 19 wherein CDR1, CDR2, and CDR3 of the LC and HC variable domain sequences are identical to respective CDRs of the BE-H9 antibody.

21. An isolated protein comprising a light chain (LC) immunoglobulin variable domain sequence and a heavy chain (HC) immunoglobulin variable domain sequence,
wherein the LC and HC variable domain sequences form an antigen binding site with affinity for the human CD44 extracellular domain, and
each of CDR1, CDR2, and CDR3 of at least one the variable domain sequences by no more than two amino acid differences from a respective CDR of the HAE-B8 antibody.

22. The protein of claim 21 wherein CDR1, CDR2, and CDR3 of the LC and HC variable domain sequences are identical to respective CDRs of the HAE-B8 antibody.

23. An isolated protein comprising a light chain (LC) immunoglobulin variable domain sequence and a heavy chain (HC) immunoglobulin variable domain sequence,
wherein the LC and HC variable domain sequences form an antigen binding site with affinity for the human CD44 extracellular domain, and
each of CDR1, CDR2, and CDR3 of at least one the variable domain sequences by no more than two amino acid differences from a respective CDR of the HAE-F1 antibody.

24. The protein of claim 23 wherein CDR1, CDR2, and CDR3 of the LC and HC variable domain sequences are identical to respective CDRs of the HAE-F1 antibody.

25. An isolated protein comprising a light chain (LC) immunoglobulin variable domain sequence and a heavy chain (HC) immunoglobulin variable domain sequence,
wherein the LC and HC variable domain sequences form an antigen binding site with affinity for the human CD44 extracellular domain, and
each of CDR 1, CDR2, and CDR3 of at least one the variable domain sequences by no more than two amino acid differences from a respective CDR of the BE-A11 antibody.

26. The protein of claim 25 wherein CDR1, CDR2, and CDR3 of the LC and HC variable domain sequences are identical to respective CDRs of the BE-A11 antibody.

27. A recombinant cell that contains one or more nucleic acids that encode the immunoglobulin variable domain sequences of the protein of claim 1.

28. A method of providing a CD44-binding antibody, the method comprising:
providing the recombinant cell of claim 27; and
maintaining the cell under conditions in which the one or more nucleic acids are expressed and the protein comprising the LC and HC immunoglobulin variable domain sequences is produced.

29. An isolated nucleic acid that comprises a first and second coding sequence,
wherein the first coding sequence encodes a first immunoglobulin chain that comprises the LC variable domain sequence of the protein of claim 1, and
the second coding sequence encodes second immunoglobulin chain that comprises the HC variable domain sequence of the protein.

30. A method of modulating activity of a CD44-expressing cell in a subject, the method comprising:
administering, to a mammalian subject, a composition that comprises the protein of claim 1, 21, 23, or 25, the composition being administered in an amount effective to modulate the activity of a CD44-expressing cell in the subject.

31. An isolated protein comprising a heavy chain immunoglobulin variable domain sequence and a light chain immunoglobulin variable domain sequence, wherein the protein binds to CD44 ectodomain with a K_{d} of less than 2×10⁻⁷ M and comprises at least two human CDRs.

32. The protein of claim 31 wherein the framework regions of the heavy and light chain variable domain are human.

33. The protein of claim 31 wherein the protein is not immunogenic in humans.

34. The protein of claim 31 wherein the protein inhibits HA binding to CD44-expressing cell KG1a cells in vitro by at least 20% inhibition at a concentration of less than 500 μg/mL.

35. The protein of claim 34 wherein the protein inhibits HA binding to a CD44-expressing cell KG1a cells in vitro by at least 50% inhibition at a concentration of less than 100 μg/mL.

36. A method of modulating activity of a CD44-expressing cell in a subject, the method comprising:
administering, to a mammalian subject, a composition that comprises the protein of claim 34, the composition being administered in an amount effective to modulate the activity of a CD44-expressing cell in the subject.

37. The method of claim 36 wherein the subject has, is predisposed to, or is diagnosed with an inflammatory disorder.

38. The method of claim 37 wherein the inflammatory disorder is rheumatoid arthritis, lupus, restenosis, graft v. host response, or multiple sclerosis.

39. The method of claim 36 wherein the subject has, is predisposed to, or is diagnosed with a neoplastic disorder.

40. The method of claim 39 wherein the neoplastic disorder is a malignant or metastatic cancer.

41. A method of preparing a subject for receiving exogenous cells, the method comprising:
administering, to a subject, the protein of claim 12, 13, 15, 17, 19, 21, 23, or 25, in an amount effective to sensitize NK cells in the subject to cell death.
